SEQUENCE LISTING (198 sequences)

SEQ ID NO: 1 - protein, 118 aa, Artificial Sequence [sequence omitted]

SEQ ID NO: 2 - protein, 8 aa, Artificial Sequence
  Description of Artificial Sequence Synthetic peptide
GFTFNNYA

SEQ ID NO: 3 - protein, 8 aa, Artificial Sequence
  Description of Artificial Sequence Synthetic peptide
ISGSGGTT

SEQ ID NO: 4 - protein, 11 aa, Artificial Sequence
  Description of Artificial Sequence Synthetic peptide
AKDSNWGNFDL

SEQ ID NO: 5 - protein, 447 aa, Artificial Sequence
  Description of Artificial Sequence Synthetic REGN727 heavy chain polypeptide
EVQLVESGGGLVQPGGSLRLSCAASGFTFNNYAMNWVRQAPGKGLDWVSTISGSGGTTNYADSVKGRFIISRDSSKHTLYLQMNSLRAEDTAVYYCAKDSNWGNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 113 aa, Artificial Sequence [sequence omitted]

SEQ ID NO: 7 - protein, 12 aa, Artificial Sequence
  Description of Artificial Sequence Synthetic peptide
QSVLYRSNNRNF

SEQ ID NO: 8 - protein, 3 aa, Artificial Sequence
  Description of Artificial Sequence Synthetic peptide
WAS

SEQ ID NO: 9 - protein, 220 aa, Artificial Sequence
  Description of Artificial Sequence Synthetic REGN727 light chain polypeptide
DIVMTQSPDSLAVSLGERATINCKSSQSVLYRSNNRNFLGWYQQKPGQPPNLLIYWASTRESGVPDRFSGSGSGTDFTLTISSLQAEDVAVYYCQQYYTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 9 aa, Artificial Sequence
  Description of Artificial Sequence Synthetic peptide
QQYYTTPYT

SEQ ID NO: 11 - protein, 127 aa, Artificial Sequence
  Description of Artificial Sequence Synthetic polypeptide
EMQLVESGGGLVQPGGSLRLSCAASGFTFSSHWMKWVRQAPGKGLEWVANINQDGSEKYYVDSVKGRFTISRDNAKNSLFLQMNSLRAEDTAVYYCARDIVLMVYDMDYYYYGMDVWGQGTTVTVSS

SEQ ID NO: 12 - protein, 8 aa, Artificial Sequence
  Description of Artificial Sequence Synthetic peptide
GFTFSSHW

SEQ ID NO: 13 - protein, 8 aa, Artificial Sequence
  Description of Artificial Sequence Synthetic peptide
INQDGSEK

SEQ ID NO: 14 - protein, 20 aa, Artificial Sequence
  Description of Artificial Sequence Synthetic peptide
ARDIVLMVYDMDYYYYGMDV

SEQ ID NO: 15 - protein, 112 aa, Artificial Sequence
  Description of Artificial Sequence Synthetic polypeptide
DIVMTQSPLSLPVTPGEPASISCRSSQSLLHSNGNNYLDWYLQKPGQSPQLLIYLGSNRASGVPDRFSGSGSGTDFTLKISRVEAEDVGVYYCMQTLQTPLTFGGGTKVEIK

SEQ ID NO: 16 - protein, 11 aa, Artificial Sequence
  Description of Artificial Sequence Synthetic peptide
QSLLHSNGNNY

SEQ ID NO: 17 - protein, 3 aa, Artificial Sequence
  Description of Artificial Sequence Synthetic peptide
LGS

SEQ ID NO: 18 - protein, 9 aa, Artificial Sequence
  Description of Artificial Sequence Synthetic peptide
MQTLQTPLT

SEQ ID NO: 19 - protein, 8 aa, Artificial Sequence
  Description of Artificial Sequence Synthetic peptide
GFTFSSHW

SEQ ID NO: 20 - protein, 8 aa, Artificial Sequence
  Description of Artificial Sequence Synthetic peptide
INQDGSEK

SEQ ID NO: 21 - protein, 20 aa, Artificial Sequence
  Description of Artificial Sequence Synthetic peptide
ARDIVLMVYDMDYYYYGMDV

SEQ ID NO: 22 - protein, 11 aa, Artificial Sequence
  Description of Artificial Sequence Synthetic peptide
QSLHHSNGNNY

SEQ ID NO: 23 - protein, 3 aa, Artificial Sequence
  Description of Artificial Sequence Synthetic peptide
LGS

SEQ ID NO: 24 - protein, 9 aa, Artificial Sequence
  Description of Artificial Sequence Synthetic peptide
MQTLQTPLT

SEQ ID NO: 25 - protein, 8 aa, Artificial Sequence
  Description of Artificial Sequence Synthetic peptide
GFTFSSHW

SEQ ID NO: 26 - protein, 8 aa, Artificial Sequence
  Description of Artificial Sequence Synthetic peptide
INQDGSEK

SEQ ID NO: 27 - protein, 20 aa, Artificial Sequence
  Description of Artificial Sequence Synthetic peptide
ARDIVLMVYHMDYYYYGMDV

SEQ ID NO: 28 - protein, 11 aa, Artificial Sequence
  Description of Artificial Sequence Synthetic peptide
QSLLHSNGNNY

SEQ ID NO: 29 - protein, 3 aa, Artificial Sequence
  Description of Artificial Sequence Synthetic peptide
LGS

SEQ ID NO: 30 - protein, 9 aa, Artificial Sequence
  Description of Artificial Sequence Synthetic peptide
MQTLQTPLT

SEQ ID NO: 31 - protein, 8 aa, Artificial Sequence
  Description of Artificial Sequence Synthetic peptide
GFTFSSHW

SEQ ID NO: 32 - protein, 8 aa, Artificial Sequence
  Description of Artificial Sequence Synthetic peptide
INQDGSEK

SEQ ID NO: 33 - protein, 20 aa, Artificial Sequence
  Description of Artificial Sequence Synthetic peptide
ARDIVLMVYHMDYYYYGMDV

SEQ ID NO: 34 - protein, 11 aa, Artificial Sequence
  Description of Artificial Sequence Synthetic peptide
QSLHHSNGNNY

SEQ ID NO: 35 - protein, 3 aa, Artificial Sequence
  Description of Artificial Sequence Synthetic peptide
LGS

SEQ ID NO: 36 - protein, 9 aa, Artificial Sequence
  Description of Artificial Sequence Synthetic peptide
MQTLQTPLT

SEQ ID NO: 37 - protein, 131 aa, Artificial Sequence
  Description of Artificial Sequence Synthetic VH; m2CX1D05 polypeptide
QVQLVQSGAEVKKPGSSVKVSCKASGGTFNSHAISWVRQAPGQGLEWMGGINPILGIANYAQKFQGRVTITADESTSTAYMELSSLRSEDTAVYYCARHYEIQIGRYGMNVYYLMYRFASWGQGTLVTVSS

SEQ ID NO: 38 - protein, 10 aa, Artificial Sequence
  Description of Artificial Sequence Synthetic VH CDR1; m2CX1D05 peptide
GGTFNSHAIS

SEQ ID NO: 39 - protein, 20 aa, Artificial Sequence
  Description of Artificial Sequence Synthetic VH CDR2; m2CX1D05 peptide
WMGGINPILGIANYAQKFQG

SEQ ID NO: 40 - protein, 22 aa, Artificial Sequence
  Description of Artificial Sequence Synthetic VH CDR3; m2CX1D05 peptide
HYEIQIGRYGMNVYYLMYRFAS

SEQ ID NO: 41 - protein, 213 aa, Artificial Sequence
  Description of Artificial Sequence Synthetic LC; m2CX1D05 polypeptide
DIQMTQSPSSLSASVGDRVTITCRASQGIRSALNWYQQKPGKAPKLLIYNGSTLQSGVPSRFSGSGSGTDFTLTISSLQPEDFAVYYCQQFDGDPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 42 - protein, 11 aa, Artificial Sequence
  Description of Artificial Sequence Synthetic VL CDR 1; m2CX1D05 peptide
RASQGIRSALN

SEQ ID NO: 43 - protein, 11 aa, Artificial Sequence
  Description of Artificial Sequence Synthetic VL CDR2; m2CX1D05 peptide
LLIYNGSTLQS

SEQ ID NO: 44 - protein, 7 aa, Artificial Sequence
  Description of Artificial Sequence Synthetic VL CDR3; m2CX1D05 peptide
QQFDGDP

SEQ ID NO: 45 - protein, 119 aa, Artificial Sequence
  Description of Artificial Sequence Synthetic VH; 1B20 polypeptide
QVQLVQSGAEVKKPGESLKISCKGSGYSFTNYWISWVRQMPGKGLEWMGIIYPGDSYTNYSPSFQGQVTISADKSISTAYLQWSSLKASDTAMYYCARDYWYKPLFDIWGQGTLVTVSS

SEQ ID NO: 46 - protein, 10 aa, Artificial Sequence
  Description of Artificial Sequence Synthetic VH CDR1; 1B20 peptide
GYSFTNYWIS

SEQ ID NO: 47 - protein, 20 aa, Artificial Sequence
  Description of Artificial Sequence Synthetic VH CDR2; 1B20 peptide
WMGIIYPGDSYTNYSPSFQG

SEQ ID NO: 48 - protein, 10 aa, Artificial Sequence
  Description of Artificial Sequence Synthetic VH CDR3; 1B20 peptide
DYWYKPLFDI

SEQ ID NO: 49 - protein, 220 aa, Artificial Sequence
  Description of Artificial Sequence Synthetic LC; 1B20 polypeptide
DIVMTQSPDSLAVSLGERATINCRSSQSVLYSSNNKNYLAWYQQKPGQPPKLLIYWASTRESGVPDRFSGSGSGTDFTLTISSLQAEDVAVYYCQQYSSFPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 50 - protein, 17 aa, Artificial Sequence
  Description of Artificial Sequence Synthetic VL CDR1; 1B20 peptide
RSSQSVLYSSNNKNYLA

SEQ ID NO: 51 - protein, 11 aa, Artificial Sequence
  Description of Artificial Sequence Synthetic VL CDR2; 1B20 peptide
LLIYWASTRES

SEQ ID NO: 52 - protein, 8 aa, Artificial Sequence
  Description of Artificial Sequence Synthetic VL CDR3; 1B20 peptide
QQYSSFPI

SEQ ID NO: 53 - protein, 120 aa, Artificial Sequence
  Description of Artificial Sequence Synthetic variable heavy antibody region polypeptide
EVQLLESGGGLVQPGGSLRLSCKASGYTFSSYGMYWVRQAPGKGLEWIGWIDPGSGGTKYNEKFKGKATISRDNSKNTLYLQMNSLRAEDTAVYYCARERYGYYFDYWGQGTLVTVSSAS

SEQ ID NO: 54 - protein, 16 aa, Artificial Sequence
  Description of Artificial Sequence Synthetic AX132 heavy chain CDR1 antibody region peptide
KASGYTFSSYGMYWVR

SEQ ID NO: 55 - protein, 23 aa, Artificial Sequence
  Description of Artificial Sequence Synthetic AX132 heavy chain CDR2 antibody region peptide
WIGWIDPGSGGTKYNEKFKGKAT

SEQ ID NO: 56 - protein, 15 aa, Artificial Sequence
  Description of Artificial Sequence Synthetic AX132 heavy chain CDR3 antibody region peptide
CARERYGYYFDYWGQ

SEQ ID NO: 57 - protein, 108 aa, Artificial Sequence
  Description of Artificial Sequence Synthetic variable light antibody region polypeptide
EIVLTQSPATLSLSPGERATITCRASQYVGSYLNWYQQKPGQAPRLLIYDASNRATGIPARFSGSGSGTDFTLTISSLEPEDFAVYYCQVWDSSPPVVFGGGTKVEIK

SEQ ID NO: 58 - protein, 17 aa, Artificial Sequence
  Description of Artificial Sequence Synthetic AX213 and AX132 light chain CDR1 antibody region peptide
ITCRASQYVGSYLNWYQ

SEQ ID NO: 59 - protein, 13 aa, Artificial Sequence
  Description of Artificial Sequence Synthetic AX213 and AX132 light chain CDR2 antibody region peptide
LIYDASNRATGIP

SEQ ID NO: 60 - protein, 16 aa, Artificial Sequence
  Description of Artificial Sequence Synthetic AX132 & AX213 light chain CDR3 antibody region peptide
YYCQVWDSSPPVVFGG

SEQ ID NO: 61 - protein, 120 aa, Artificial Sequence
  Description of Artificial Sequence Synthetic variable heavy antibody region polypeptide
EVQLLESGGGLVQPGGSLRLSCKASGYTFSRYGINWVRQAPGKGLEWIGRIDPGNGGTRYNEKFKGKATISRDNSKNTLYLQMNSLRAEDTAVYYCARANDGYSFDYWGQGTLVTVSSAS

SEQ ID NO: 62 - protein, 16 aa, Artificial Sequence
  Description of Artificial Sequence Synthetic AX213 heavy chain CDR1 antibody region peptide
KASGYTFSRYGINWVR

SEQ ID NO: 63 - protein, 23 aa, Artificial Sequence
  Description of Artificial Sequence Synthetic AX213 heavy chain CDR2 antibody region peptide
WIGRIDPGNGGTRYNEKFKGKAT

SEQ ID NO: 64 - protein, 15 aa, Artificial Sequence
  Description of Artificial Sequence Synthetic AX213 heavy chain CDR3 antibody region peptide
CARANDGYSFDYWGQ

SEQ ID NO: 65 - protein, 108 aa, Artificial Sequence; identical to SEQ ID NO: 57
  Description of Artificial Sequence Synthetic variable light antibody region polypeptide

SEQ ID NO: 66 - protein, 17 aa, Artificial Sequence
  Description of Artificial Sequence Synthetic AX213 and AX132 light chain CDR1 antibody region peptide
ITCRASQYVGSYLNWYQ

SEQ ID NO: 67 - protein, 13 aa, Artificial Sequence
  Description of Artificial Sequence Synthetic AX213 and AX132 light chain CDR2 antibody region peptide
LIYDASNRATGIP

SEQ ID NO: 68 - protein, 16 aa, Artificial Sequence
  Description of Artificial Sequence Synthetic AX132 & AX213 light chain CDR3 antibody region peptide
YYCQVWDSSPPVVFGG

SEQ ID NO: 69 - protein, 119 aa, Artificial Sequence
  Description of Artificial Sequence Synthetic AX1 VH antibody sequence polypeptide
EVQLLESGGGLVQPGGSLRLSCKASGFTFTSYYMHWVRQAPGKGLEWIGRINPDSGSTKYNEKFKGRATISRDNSKNTLYLQMNSLRAEDTAVYYCARGGRLSWDFDVWGQGTLVTVSS

SEQ ID NO: 70 - protein, 16 aa, Artificial Sequence
  Description of Artificial Sequence Synthetic AX1 VH CDR1 antibody sequence peptide
KASGFTFTSYYMHWVR

SEQ ID NO: 71 - protein, 23 aa, Artificial Sequence
  Description of Artificial Sequence Synthetic AX1 VH CDR2 antibody sequence peptide
WIGRINPDSGSTKYNEKFKGRAT

SEQ ID NO: 72 - protein, 16 aa, Artificial Sequence
  Description of Artificial Sequence Synthetic AX1 VH CDR3 antibody sequence peptide
CARGGRLSWDFDVWGQ

SEQ ID NO: 73 - protein, 109 aa, Artificial Sequence
  Description of Artificial Sequence Synthetic AX1 VL antibody sequence polypeptide
DIQMTQSPSSLSASVGDRVTITCRASQDISRYLAWYQQKPGKAPKLLIYAASSLQSGVPSRFSGSGSGTDFTLTISSLQPEDFATYYCAAYDYSLGGYVFGDGTKVEIK

SEQ ID NO: 74 - protein, 11 aa, Artificial Sequence
  Description of Artificial Sequence Synthetic AX1 VL CDR1 antibody sequence peptide
RASQDISRYLA

SEQ ID NO: 75 - protein, 7 aa, Artificial Sequence
  Description of Artificial Sequence Synthetic AX1 AX9 AX189 VL CDR2 antibody sequence peptide
AASSLQS

SEQ ID NO: 76 - protein, 11 aa, Artificial Sequence
  Description of Artificial Sequence Synthetic AX1 VL CDR3 antibody sequence peptide
AAYDYSLGGYV

SEQ ID NO: 77 - protein, 121 aa, Artificial Sequence
  Description of Artificial Sequence Synthetic AX9 AX189 VH antibody sequence polypeptide
EVQLLESGGGLVQPGGSLRLSCKASGYTFSSYWMHWVRQAPGKGLEWIGRIDPYNGGTKYNEKFKGKATISRDNSKNTLYLQMNSLRAEDTAVYYCARYGYYLGSYAMDYWGQGTLVTVSS

SEQ ID NO: 78 - protein, 16 aa, Artificial Sequence
  Description of Artificial Sequence Synthetic AX9 AX189 VH CDR1 antibody sequence peptide
KASGYTFSSYWMHWVR

SEQ ID NO: 79 - protein, 23 aa, Artificial Sequence
  Description of Artificial Sequence Synthetic AX9 AX189 VH CDR2 antibody sequence peptide
WIGRIDPYNGGTKYNEKFKGKAT

SEQ ID NO: 80 - protein, 18 aa, Artificial Sequence
  Description of Artificial Sequence Synthetic AX9 AX189 VH CDR3 antibody sequence peptide
CARYGYYLGSYAMDYWGQ

SEQ ID NO: 81 - protein, 109 aa, Artificial Sequence
  Description of Artificial Sequence Synthetic AX189 VL antibody sequence polypeptide
DIQMTQSPSSLSASVGDRVTITCRASQDVSRYLTWYQQKPGKAPKLLIYAASSLQSGVPSRFSGSGSGTDFTLTISSLQPEDFATYYCQAYDYSLSGYVFGGGTKVEIK

SEQ ID NO: 82 - protein, 11 aa, Artificial Sequence
  Description of Artificial Sequence Synthetic AX189 VL CDR1 antibody sequence peptide
RASQDVSRYLT

SEQ ID NO: 83 - protein, 7 aa, Artificial Sequence
  Description of Artificial Sequence Synthetic AX1 AX9 AX189 VL CDR2 antibody sequence peptide
AASSLQS

SEQ ID NO: 84 - protein, 11 aa, Artificial Sequence
  Description of Artificial Sequence Synthetic AX189 VL CDR3 antibody sequence peptide
QAYDYSLSGYV

SEQ ID NO: 85 - protein, 115 aa, Homo sapiens
QVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSS

SEQ ID NO: 86 - protein, 5 aa, Homo sapiens
SYGIS

SEQ ID NO: 87 - protein, 17 aa, Homo sapiens
WVSFYNGNTNYAQKLQG

SEQ ID NO: 88 - protein, 6 aa, Homo sapiens
GYGMDV

SEQ ID NO: 89 - protein, 109 aa, Homo sapiens
QSALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVL

SEQ ID NO: 90 - protein, 14 aa, Homo sapiens
TGTSSDVGGYNSVS

SEQ ID NO: 91 - protein, 7 aa, Homo sapiens
EVSNRPS

SEQ ID NO: 92 - protein, 9 aa, Homo sapiens
NSYTSTSMV

SEQ ID NO: 93 - protein, 123 aa, Homo sapiens
EVQLVESGGGLVKPGGSLRLSCAASGFTFSSYSMNWVRQAPGKGLEWVSSISSSSSYISYADSVKGRFTISRDNAKNSLYLQMNSLRAEDTAVYFCARDYDFWSAYYDAFDVWGQGTMVTVSS

SEQ ID NO: 94 - protein, 10 aa, Homo sapiens
GFTFSSYSMN

SEQ ID NO: 95 - protein, 17 aa, Homo sapiens
SISSSSSYISYADSVKG

SEQ ID NO: 96 - protein, 14 aa, Homo sapiens
DYDFWSAYYDAFDV

SEQ ID NO: 97 - protein, 111 aa, Homo sapiens
QSVLTQPPSVSGAPGQRVTISCTGSSSNIGAGYDVHWYQQLPGTAPKLLISGNSNRPSGVPDRFSGSKSGTSASLAITGLQAEDEADYYCQSYDSSLSGSVFGGGTKLTVL

SEQ ID NO: 98 - protein, 14 aa, Homo sapiens
TGSSSNIGAGYDVH

SEQ ID NO: 99 - protein, 7 aa, Homo sapiens
GNSNRPS

SEQ ID NO: 100 - protein, 11 aa, Homo sapiens
QSYDSSLSGSV

SEQ ID NO: 101 - protein, 114 aa, Homo sapiens
QVQLVESGGGVAQPGRSLRLSCAASGFTFSSYGMHWVRQAPGKGLEWVAVIYYDGINKHYADSVKGRFTISRDNSKNTLYLQMNSLRAEDTAVYYCARDRGLDWGQGTLVTVSS

SEQ ID NO: 102 - protein, 10 aa, Homo sapiens
GFTFSSYGMH

SEQ ID NO: 103 - protein, 17 aa, Homo sapiens
VIYYDGINKHYADSVKG

SEQ ID NO: 104 - protein, 5 aa, Homo sapiens
DRGLD

SEQ ID NO: 105 - protein, 113 aa, Homo sapiens
DIVMTQSPDSLAVSLGERATINCKSSQSVLYSSNSKNYLVWYQQKPGQPPKLLIYWASTRESGVPDRFSGSGSGTDFTLTISSLQAEDVAVYYCQQYYSTPWTFGQGTKVEIK

SEQ ID NO: 106 - protein, 17 aa, Homo sapiens
KSSQSVLYSSNSKNYLV

SEQ ID NO: 107 - protein, 7 aa, Homo sapiens
WASTRES

SEQ ID NO: 108 - protein, 9 aa, Homo sapiens
QQYYSTPWT

SEQ ID NO: 109 - protein, 118 aa, Artificial Sequence
  Description of Artificial Sequence Synthetic anti-PCSK9 monoclonal antibody pJG04 (clones LGT-209 and LGT-210) Vh heavy chain variable region (FR1-FR4) polypeptide
QVQLVQSGAEVKKPGASVKVSCKASGYTFSTMYMSWVRQAPGQGLEWMGRIDPANEHTNYAQKFQGRVTMTRDTSISTAYMELSRLTSDDTAVYYCARSYYYYNMDYWGQGTLVTVSS

SEQ ID NO: 110 - protein, 5 aa, Artificial Sequence
  Description of Artificial Sequence Synthetic anti-PCSK9 monoclonal antibody clones LGT-209, LGT-210 and LGT-211 heavy chain CDR1 peptide
TMYMS

SEQ ID NO: 111 - protein, 17 aa, Artificial Sequence
  Description of Artificial Sequence Synthetic anti-PCSK9 monoclonal antibody clones LGT-209, LGT-210 and LGT-211 heavy chain CDR2 peptide
RIDPANEHTNYAQKFQG

SEQ ID NO: 112 - protein, 9 aa, Artificial Sequence
  Description of Artificial Sequence Synthetic anti-PCSK9 monoclonal antibody pJG04(clones LGT-209 and LGT-210) Vh heavy chain complementarity determining region 3 (CDR3) peptide
SYYYYNMDY

SEQ ID NO: 113 - protein, 106 aa, Artificial Sequence
  Description of Artificial Sequence Synthetic anti-PCSK9 monoclonal antibody pJG10(clones LGT-209 and LGT-211) Vk light chain variable region (FR1-FR4) polypeptide
QIVLTQSPATLSVSPGERATLSCRASQSVSYMHWYQQKPGQAPRLLIYGVFRRATGIPDRFSGSGSGTDFTLTIGRLEPEDFAVYYCLQWSSDPPTFGQGTKLEIK

SEQ ID NO: 114 - protein, 10 aa, Artificial Sequence
  Description of Artificial Sequence Synthetic anti-PCSK9 monoclonal antibody clones LGT-209, LGT-210 and LGT-211 light chain CDR1 peptide
RASQSVSYMH

SEQ ID NO: 115 - protein, 7 aa, Artificial Sequence
  Description of Artificial Sequence Synthetic anti-PCSK9 monoclonal antibody clones LGT-209, LGT-210 and LGT-211 light chain CDR1 peptide
GVFRRAT

SEQ ID NO: 116 - protein, 9 aa, Artificial Sequence
  Description of Artificial Sequence Synthetic mouse anti-PCSK9 monoclonal antibody LFU720 and anti-PCSK9 monoclonal antibody clones LGT-209, LGT-210 and LGT-211 light chain CDR3 peptide
LQWSSDPPT

SEQ ID NO: 117 - protein, 118 aa, Homo sapiens
QVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSS

SEQ ID NO: 118 - protein, 7 aa, Artificial Sequence
  Description of Artificial Sequence Synthetic peptide
GYTFTSY

SEQ ID NO: 119 - protein, 6 aa, Artificial Sequence
  Description of Artificial Sequence Synthetic peptide
SPFGGR

SEQ ID NO: 120 - protein, 9 aa, Artificial Sequence
  Description of Artificial Sequence Synthetic variable heavy chain CDR peptide
ERPLYASDL

SEQ ID NO: 121 - protein, 107 aa, Homo sapiens
DIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIK

SEQ ID NO: 122 - protein, 11 aa, Artificial Sequence
  Description of Artificial Sequence Synthetic variable light chain CDR peptide
RASQGISSALA

SEQ ID NO: 123 - protein, 7 aa, Artificial Sequence
  Description of Artificial Sequence Synthetic variable light chain CDR peptide
SASYRYT

SEQ ID NO: 124 - protein, 9 aa, Artificial Sequence
  Description of Artificial Sequence Synthetic variable light chain CDR peptide
QQRYSLWRT

SEQ ID NO: 125 - protein, 118 aa, Homo sapiens
QVQLVQSGAEVKKPGASVKVSCKASGYTFTSYYMHWVRQAPGQGLEWMGEIHPSGGRTNYNEKFKSRVTMTRDTSTSTVYMELSSLRSEDTAVYYCARERPLYAMDYWGQGTTVTVSS

SEQ ID NO: 126 - protein, 10 aa, Homo sapiens
GYTFTSYYMH

SEQ ID NO: 127 - protein, 17 aa, Homo sapiens
EIHPSGGRTNYNEKFKS

SEQ ID NO: 128 - protein, 9 aa, Homo sapiens
ERPLYAMDY

SEQ ID NO: 129 - protein, 107 aa, Homo sapiens [sequence omitted]

SEQ ID NO: 130 - protein, 11 aa, Homo sapiens
KASQDVHTAVA

SEQ ID NO: 131 - protein, 7 aa, Homo sapiens
HASYRYT

SEQ ID NO: 132 - protein, 9 aa, Homo sapiens
QQRYSLWRT

SEQ ID NO: 133 - protein, 118 aa, Homo sapiens [sequence omitted]

SEQ ID NO: 134 - protein, 10 aa, Homo sapiens
GYTFTSYYMH

SEQ ID NO: 135 - protein, 17 aa, Homo sapiens
EIHPSGGRTNYNEKFKS

SEQ ID NO: 136 - protein, 9 aa, Homo sapiens
ERPLYASDL

SEQ ID NO: 137 - protein, 107 aa, Homo sapiens [sequence omitted]

SEQ ID NO: 138 - protein, 11 aa, Homo sapiens
KASQDVHTAVA

SEQ ID NO: 139 - protein, 7 aa, Homo sapiens
HASYRYT

SEQ ID NO: 140 - protein, 9 aa, Homo sapiens
QQRYSLWRT

SEQ ID NO: 141 - protein, 118 aa, Mus musculus
QVQLQQPGAELVKPGASVKLSCKASGYTFTSYWMHWVKQRPGQGLEWIGEINPSNGRTNYNEKFKSKATLTVDKSSSTAYMQLSSLTSEDSAVYYCARERPLYAMDYWGQGTSVTVSS

SEQ ID NO: 142 - protein, 7 aa, Mus musculus
GYTFTSY

SEQ ID NO: 143 - protein, 6 aa, Mus musculus
NPSNGR

SEQ ID NO: 144 - protein, 9 aa, Mus musculus
ERPLYAMDY

SEQ ID NO: 145 - protein, 108 aa, Mus musculus
DIVMTQSHKFMSTSVGDRVSITCKASQDVSTAVAWYQQKPGQSPKLLIYSASYRYTGVPDRFTGSGSGTDFTFTISSVQAEDLAVYYCQQRYSTPRTFGGGTKLEIKR

SEQ ID NO: 146 - protein, 11 aa, Mus musculus
KASQDVSTAVA

SEQ ID NO: 147 - protein, 7 aa, Artificial Sequence
  Description of Artificial Sequence Synthetic variable light chain CDR peptide
SASYRYT

SEQ ID NO: 148 - protein, 9 aa, Mus musculus
QQRYSTPRT

SEQ ID NO: 149 - protein, 115 aa, Mus musculus
EVQLQQSGPELVKPGASVKISCKASGYTFTDYYMNWVKQSHGKSLEWIGDINPNNGGTTYNQKFKGKATLTVDKSYSTAYMELRSLTSEDSAVYYCARWLLFAYWGQGTLVTVSA

SEQ ID NO: 150 - protein, 7 aa, Mus musculus
GYTFTDY

SEQ ID NO: 151 - protein, 6 aa, Mus musculus
NPNNGG

SEQ ID NO: 152 - protein, 6 aa, Mus musculus
WLLFAY

SEQ ID NO: 153 - protein, 108 aa, Mus musculus
DIVMTQSQKFMSTSVGDRVSVTCKASQNVGTNVAWYQQKPGQSPKALIYSASYRYSGVPDRFTGSGSGTDFTLTISNVLSEDLAEYFCQQFYSYPYTFGGGTKLEIKR

SEQ ID NO: 154 - protein, 11 aa, Mus musculus
KASQNVGTNVA

SEQ ID NO: 155 - protein, 7 aa, Mus musculus
SASYRYS

SEQ ID NO: 156 - protein, 9 aa, Mus musculus
QQFYSYPYT

SEQ ID NO: 157 - protein, 123 aa, Mus musculus
EVQLQQSGPELVKPGASVKISCKASGYTFTDYYMNWVKQSHGKSLEWIGDINPNNGGTSYNQKFKGKATLTVDKSSSTAYMELRSLTSEDSAVYYCAGGGIYYRYDRNYFDYWGQGTTLTVSS

SEQ ID NO: 158 - protein, 7 aa, Mus musculus
GYTFTDY

SEQ ID NO: 159 - protein, 6 aa, Mus musculus
NPNNGG

SEQ ID NO: 160 - protein, 14 aa, Mus musculus
GGIYYRYDRNYFDY

SEQ ID NO: 161 - protein, 107 aa, Mus musculus
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPFTFGSGTKLEIK

SEQ ID NO: 162 - protein, 11 aa, Mus musculus
SASQGISNYLN

SEQ ID NO: 163 - protein, 7 aa, Mus musculus
YTSSLHS

SEQ ID NO: 164 - protein, 9 aa, Artificial Sequence
  Description of Artificial Sequence Synthetic peptide
QQYSKLPFT

SEQ ID NO: 165 - protein, 117 aa, Mus musculus
EVKLVESEGGLVQPGSSMKLSCTASGFTFSDYYMAWVRQVPEKGLEWVANINYDGSNTSYLDSLKSRFIISRDNAKNILYLQMSSLKSEDTATYYCAREKFAAMDYWGQGTSVTVSS

SEQ ID NO: 166 - protein, 7 aa, Mus musculus
GFTFSDY

SEQ ID NO: 167 - protein, 6 aa, Mus musculus
NYDGSN

SEQ ID NO: 168 - protein, 8 aa, Mus musculus
EKFAAMDY

SEQ ID NO: 169 - protein, 108 aa, Mus musculus
DIVMTQSHKFMSTSFGDRVSITCKASQDVSNALAWYQQKPGHSPKLLIFSASYRYTGVPDRFTGSGSGTDFTFTISSVQAEDLAVYYCQQHYSTPWTFGGGTKLEIKR

SEQ ID NO: 170 - protein, 11 aa, Mus musculus
KASQDVSNALA

SEQ ID NO: 171 - protein, 7 aa, Artificial Sequence
  Description of Artificial Sequence Synthetic variable light chain CDR peptide
SASYRYT

SEQ ID NO: 172 - protein, 9 aa, Mus musculus
QQHYSTPWT

SEQ ID NO: 173 - protein, 121 aa, Artificial Sequence
  Description of Artificial Sequence Synthetic polypeptide
EVQLVESGGGLVQPGGSLRLSCAASGFTFTRHTIHWVRQAPGKGLEWVARISPANGNTNYADSVKGRFTISADTSKNTAYLQMNSLRAEDTAVYYCARWIGSRELYIMDYWGQGTLVTVSS

SEQ ID NO: 174 - protein, 10 aa, Artificial Sequence
  Description of Artificial Sequence Synthetic peptide
GFTFTRHTIH

SEQ ID NO: 175 - protein, 17 aa, Artificial Sequence
  Description of Artificial Sequence Synthetic peptide
RISPANGNTNYADSVKG

SEQ ID NO: 176 - protein, 12 aa, Artificial Sequence
  Description of Artificial Sequence Synthetic peptide
WIGSRELYIMDY

SEQ ID NO: 177 - protein, 108 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYRIQPTFGQGTKVEIKR

SEQ ID NO: 178 - protein, 11 aa, Artificial Sequence
  Description of Artificial Sequence Synthetic peptide
RASQDVSTAVA

SEQ ID NO: 179 - protein, 7 aa, Artificial Sequence
  Description of Artificial Sequence Synthetic peptide
SASFLYS

SEQ ID NO: 180 - protein, 9 aa, Artificial Sequence
  Description of Artificial Sequence Synthetic peptide
QQSYRIQPT

SEQ ID NO: 181 - protein, 121 aa, Artificial Sequence
  Description of Artificial Sequence Synthetic polypeptide
EVQLVESGGGLVQPGGSLRLSCAASGFTFSSTAIHWVRQAPGKGLEWVARISPANGNTNYADSVKGRFTISADTSKNTAYLQMNSLRAEDTAVYYCARWIGSRELYIMDYWGQGTLVTVSS

SEQ ID NO: 182 - protein, 10 aa, Artificial Sequence
  Description of Artificial Sequence Synthetic peptide
GFTFSSTAIH

SEQ ID NO: 183 - protein, 17 aa, Artificial Sequence
  Description of Artificial Sequence Synthetic peptide
RISPANGNTNYADSVKG

SEQ ID NO: 184 - protein, 12 aa, Artificial Sequence
  Description of Artificial Sequence Synthetic peptide
WIGSRELYIMDY

SEQ ID NO: 185 - protein, 108 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYPALHTFGQGTKVEIKR

SEQ ID NO: 186 - protein, 11 aa, Artificial Sequence
  Description of Artificial Sequence Synthetic peptide
RASQDVSTAVA

SEQ ID NO: 187 - protein, 7 aa, Artificial Sequence
  Description of Artificial Sequence Synthetic peptide
SASFLYS

SEQ ID NO: 188 - protein, 9 aa, Artificial Sequence
  Description of Artificial Sequence Synthetic peptide
QQSYPALHT

SEQ ID NO: 189 - protein, 125 aa, Artificial Sequence
  Description of Artificial Sequence Synthetic polypeptide
EVQLVESGGGLVKPGGSLRLSCAASGFPFSKLGMVWVRQAPGKGLEWVSTISSGGGYTYYPDSVKGRFTISRDNAKNSLYLQMNSLRAEDTAVYYCAREGISFQGGTYTYVMDYWGQGTLVTVSS

SEQ ID NO: 190 - protein, 10 aa, Artificial Sequence
  Description of Artificial Sequence Synthetic peptide
GFPFSKLGMV

SEQ ID NO: 191 - protein, 17 aa, Artificial Sequence
  Description of Artificial Sequence Synthetic peptide
TISSGGGYTYYPDSVKG

SEQ ID NO: 192 - protein, 16 aa, Artificial Sequence
  Description of Artificial Sequence Synthetic peptide
EGISFQGGTYTYVMDY

SEQ ID NO: 193 - protein, 112 aa, Artificial Sequence
  Description of Artificial Sequence Synthetic polypeptide
DIVMTQSPLSLPVTPGEPASISCRSSKSLLHRNGITYSYWYLQKPGQSPQLLIYQLSNLASGVPDRFSGSGSGTDFTLKISRVEAEDVGVYYCYQNLELPLTFGQGTKVEIK

SEQ ID NO: 194 - protein, 16 aa, Artificial Sequence
  Description of Artificial Sequence Synthetic peptide
RSSKSLLHRNGITYSY

SEQ ID NO: 195 - protein, 7 aa, Artificial Sequence
  Description of Artificial Sequence Synthetic peptide
QLSNLAS

SEQ ID NO: 196 - protein, 9 aa, Artificial Sequence
  Description of Artificial Sequence Synthetic peptide
YQNLELPLT

SEQ ID NO: 197 - DNA, 2076 bp, Homo sapiens
atgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccag

SEQ ID NO: 198 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

(19) United States
(12) Patent Application Publication
(10) Pub. No.: US 2019/0031774 A1
(43) Pub. Date: Jan. 31, 2019

(54) METHODS FOR TREATING HYPERLIPIDEMIA IN DIABETIC PATIENTS BY ADMINISTERING A PCSK9 INHIBITOR

(71) Applicant: SANOFI BIOTECHNOLOGY, Paris (FR)

(72) Inventor: Maja Bujas-Bobanovic, Paris (FR)

(21) Appl. No.: 16/004,126

(22) Filed: Jun. 8, 2018

Related U.S. Application Data
(60) Provisional application No. 62/517,672, filed on Jun. 9, 2017.
(60) Provisional application No. 62/532,162, filed on Jul. 13, 2017.

(30) Foreign Application Priority Data
May 4, 2018 (EP) 18305565.6

(51) Int. Cl. C07K 16/40; A61P 3/10; A61K 9/00

(52) U.S. Cl. CPC C07K 16/40; A61P 3/10; A61K 45/06; C12Y 304/21062; A61K 2300/00; A61K 9/0019

(57) ABSTRACT

Provided are methods for treating high cardiovascular risk patients with hypercholesterolemia and type 1 or type 2 diabetes mellitus receiving insulin therapy. These methods generally comprise administering to a patient a pharmaceutical composition comprising an antibody or antigen binding fragment, thereof, which specifically binds hPCSK9 antibody, in combination with insulin therapy.

### RELATED APPLICATIONS

This application claims the benefit of priority to U.S. Provisional Patent Application No. 62/517,672, filed Jun. 9, 2017, U.S. Provisional Patent Application No. 62/532,162, filed Jul. 13, 2017, and European Patent Application No. 18305565.6, filed May 4, 2018. The contents of each of these related applications are hereby incorporated by reference in their entireties.

### FIELD OF THE INVENTION

The present invention relates to the field of therapeutic treatments of diseases and disorders which are associated with elevated levels of lipids and lipoproteins. More specifically, the invention relates to the use of PCSK9 inhibitors to treat diabetic patients with hyperlipidemia, including hypercholesterolemia.

### BACKGROUND

Hyperlipidemia is a general term that encompasses diseases and disorders characterized by or associated with elevated levels of lipids and/or lipoproteins in the blood. Hyperlipidemias include hypercholesterolemia, hypertriglyceridemia, combined hyperlipidemia, and elevated lipoprotein a (Lp(a)). A particular prevalent form of hyperlipidemia in many populations is hypercholesterolemia.

Hypercholesterolemia, particularly an increase in low-density lipoprotein (LDL) cholesterol (LDL-C) levels, constitutes a major risk for the development of atherosclerosis and coronary heart disease (CHD) (Sharrett et al., 2001, Circulation 104:1108-1113). Low-density lipoprotein cholesterol is identified as the primary target of cholesterol lowering therapy and is accepted as a valid surrogate therapeutic endpoint. Numerous studies have demonstrated that reducing LDL-C levels reduces the risk of CHD with a strong direct relationship between LDL-C levels and CHD events; for each 1 mmol/L (˜40 mg/dL) reduction in LDL-C, cardiovascular disease (CVD) mortality and morbidity is lowered by 22%. Greater reductions in LDL-C produce greater reduction in CHD events, and comparative data of intensive versus standard statin treatment suggest that the lower the LDL-C level, the greater the benefit in patients at very high cardiovascular (CV) risk.

Cardiovascular disease (CVD) is a major cause of morbidity and mortality in patients with type 1 (T1) or type 2 (T2) diabetes mellitus (DM), and insulin-treated diabetic patients have an even higher CV risk. Furthermore, the presence of comorbid DM among those who have atherosclerotic CVD (ASCVD) significantly increases the risk of CV events. Several studies and meta-analyses have shown that lowering LDL-C using statins leads to significant reductions in CV events in DM patients, with further CV risk reduction associated with additional LDL-C-lowering using concomitant ezetimibe. However, even with the currently available treatments, many patients with DM continue to have persistent lipid abnormalities and are therefore exposed to a residual risk of CV events.

Current LDL-C-lowering medications include proprotein convertase subtilisin/kexin type 9 (PCSK9) inhibitors, such as anti-PCSK9 antibodies. Although anti-PCSK9 antibodies have undergone extensive clinical investigation, the efficacy and safety of alirocumab in the diabetic population are not fully understood. Thus, there is a need in the art to identify treatment regimens for anti-PCSK9 antibodies that provide optimal efficacy and safety for the treatment of hypercholesterolemia in diabetic patients receiving insulin therapy who are at high CV risk.

### BRIEF SUMMARY OF THE INVENTION

The instant disclosure provides methods for treating hypercholesterolemia in a patient with diabetes mellitus (DM) receiving insulin therapy. In certain embodiments, the methods comprise administering one or more doses of an antibody or an antigen-binding fragment thereof which specifically binds human PCSK9 to a patient with hypercholesterolemia and diabetes. In certain embodiments, the patient has a high cardiovascular risk. In certain embodiments, the patient receives a concomitant anti-diabetic therapy in addition to insulin therapy.

According to one aspect, the methods comprise a method for treating hypercholesterolemia in a patient with type 1 diabetes mellitus (T1 DM), the method comprising: (a) selecting a high cardiovascular risk patient receiving insulin therapy that has (i) T1 DM, and (ii) hypercholesterolemia not adequately controlled by maximally tolerated statin therapy; and (b) administering to the patient 75 mg, 150 mg, or 300 mg of an antibody or an antigen-binding fragment thereof which specifically binds human proprotein convertase subtilisin/kexin type 9 (PCSK9), wherein the patient receives a concomitant insulin therapy.

In certain embodiments, 75 mg of the antibody or antigen binding fragment is administered to the patient every two weeks. In other embodiments, 150 mg of the antibody or antigen binding fragment is administered to the patient every two weeks. In other embodiments, 300 mg of the antibody or antigen binding fragment is administered to the patient every four weeks.

In certain embodiments, the antibody or antigen-binding fragment thereof comprises the heavy and light chain CDRs of a HCVR/LCVR amino acid sequence pair comprising SEQ ID NOs: 1/6. In certain embodiments, the antibody or antigen-binding fragment thereof comprises the three heavy chain CDRs set forth in SEQ ID NOs: 2, 3, and 4, and the three light chain CDRs set forth in SEQ ID NOs: 7, 8, and 10. In certain embodiments, the antibody or antigen-binding fragment thereof comprises a heavy chain variable region (HCVR) having the amino acid sequence of SEQ ID NO: 1 and a light chain variable region (LCVR) having the amino acid sequence of SEQ ID NO: 6. In certain embodiments, the antibody or antigen-binding fragment thereof competes for binding with an antibody or antigen-binding fragment thereof that comprises an HCVR having the amino acid sequence of SEQ ID NO: 1 and an LCVR having the amino acid sequence of SEQ ID NO: 6. In certain embodiments, the antibody or antigen-binding fragment thereof binds to the same epitope on PCSK9 as an antibody comprising an HCVR having the amino acid sequence of SEQ ID NO: 1 and an LCVR having the amino acid sequence of SEQ ID NO: 6. In certain embodiments, the antibody or antigen-binding fragment thereof binds to an epitope on PCSK9 that overlaps with the epitope of an antibody comprising an HCVR having the amino acid sequence of SEQ ID NO: 1 and an LCVR having the amino acid sequence of SEQ ID NO: 6.

In certain embodiments, the antibody or antigen-binding fragment thereof comprises heavy and light chain CDR amino acid sequences having SEQ ID NOs: 86, 87, 88, 90, 91, and 92. In certain embodiments, the antibody or antigen-binding fragment thereof comprises an HCVR having an amino acid sequence at least 90%, 95%, or 99% identical to the amino acid sequence set forth in SEQ ID NO:85, and an LCVR having an amino acid sequence at least 90%, 95%, or 99% identical to the amino acid sequence set forth in SEQ ID NO:89.

In certain embodiments, the antibody or antigen-binding fragment thereof is selected from the group consisting of alirocumab, evolocumab, bococizumab, lodelcizumab, ralpancizumab and LY3015014. In certain embodiments, the antibody or antigen-binding fragment thereof is alirocumab.

In certain embodiments, the method disclosed herein further comprises (c) administering to the patient one or more following doses of 75 mg of the antibody or antigen-binding fragment thereof about every two weeks if, e.g., after 8 weeks, the LDL-C level in the patient is lower than the threshold level, or administering one or more following doses of 150 mg of the antibody or antigen-binding fragment thereof about every two weeks if, e.g., after 8 weeks, the LDL-C level in the patient is greater than or equal to the threshold level. In certain embodiments, the threshold level is 70 mg/dL.

In certain embodiments, the method disclosed herein further comprises (c) administering to the patient one or more following doses of 300 mg of the antibody or antigen-binding fragment thereof about every four weeks if, e.g., after 8 weeks, the LDL-C level in the patient is lower than the threshold level, or administering one or more following doses of 150 mg of the antibody or antigen-binding fragment thereof about every two weeks if, e.g., after 8 weeks, the LDL-C level in the patient is greater than or equal to the threshold level. In certain embodiments, the threshold level is 70 mg/dL.

In certain embodiments, the antibody or antigen-binding fragment thereof is administered subcutaneously.

In certain embodiments, the patient further receives a concomitant lipid-modifying therapy (LMT). In certain embodiments, the LMT is selected from the group consisting of a statin, a cholesterol absorption inhibitor, a fibrate, niacin, an omega-3 fatty acid, and a bile acid sequestrant. In certain embodiments, the LMT is a statin therapy. In certain embodiments, the statin is selected from the group consisting of atorvastatin, rosuvastatin, simvastatin, pravastatin, lovastatin, fluvastatin, pitavastatin, and cerivastatin. In certain embodiments, the statin therapy is a maximally tolerated dose statin therapy. In certain embodiments, the cholesterol absorption inhibitor is ezetimibe. In certain embodiments, the patient is intolerant to a statin.

In certain embodiments, the insulin therapy is selected from the group consisting of human insulin, Insulin glargine, insulin glulisine, insulin detemir, insulin lispro, insulin degludec, insulin aspart, and basal insulin, In certain embodiments, the patient further receives a further concomitant anti-diabetic therapy in addition to insulin therapy. In certain embodiments, the additional anti-diabetic therapy is selected from the group consisting of a glucagon like peptide 1 (GLP-1) therapy, a gastrointestinal peptide, a glucagon receptor agonist or antagonist, a glucose-dependent insulinotropic polypeptide (GIP) receptor agonist or antagonist, a ghrelin antagonist or inverse agonist, xenin, a xenin analogue, a biguanide, a sulfonylurea, a meglitinide, a thiazolidinedione, a DPP-4 inhibitor, an alpha-glucosidase inhibitor, a sodium-dependent glucose transporter 2 (SGLT-2) inhibitor, an SGLT-1 inhibitor, a peroxisome proliferator-activated receptor (PPAR−)(alpha, gamma or alpha/gamma) agonist or modulator, amylin, an amylin analogue, a G-protein coupled receptor 119 (GPR119) agonist, a GPR40 agonist, a GPR120 agonist, a GPR142 agonist, a systemic or low-absorbable TGR5 agonist, a diabetes immunotherapeutic, an anti-inflammatory agents for the treatment of the metabolic syndrome and diabetes, an adenosine monophosphate-activated protein kinase (AMPK) stimulant, an inhibitor of 11-beta-hydroxysteroid dehydrogenase 1, an activator of glucokinase, an inhibitor of diacylglycerol O-acyltransferase (DGAT), a modulator of glucose transporter-4, a somatostatin receptor 3 agonist, a lipid lowering agent, and a combination thereof.

In certain embodiments, the antibody or antigen-binding fragment thereof reduces the LDL-C level of the patient, e.g., by at least 30%, 35%, 40%, or 45%. In certain embodiments, the antibody or antigen-binding fragment thereof reduces the non-HDL-C level of the patient, e.g., by at least 25%, 30%, 35%, or 40%. In certain embodiments, the antibody or antigen-binding fragment thereof reduces the apolipoprotein C3 (ApoC3) level of the patient (e.g., by at least about 6.0%, about 6.5%, about 7.0% or about 7.5% after 12 or 24 weeks of treatment). In certain embodiments, the antibody or antigen-binding fragment thereof reduces the number of lipoprotein particles in the patient (e.g., by at least about 20%, about 30%, about 40% or about 50%% after 12 or 24 weeks of treatment). In other embodiments, the antibody or antigen-binding fragment thereof reduces the size of lipoprotein particles in the patient (e.g., by at least about 1.5%, about 2%, about 2.5%, or about 3% after 12 or 24 weeks of treatment).

In certain embodiments, the antibody or antigen-binding fragment thereof (a) does not affect the hemoglobin A1c (HbA1c) level of the patient; and/or (b) does not affect the fasting plasma glucose (FPG) level of the patient.

According to another aspect, the methods comprise a method for treating hypercholesterolemia in a patient with type 1 diabetes mellitus (T1 DM), the method comprising:

(a) selecting a high cardiovascular risk patient receiving insulin therapy that has (i) T1 DM, and (ii) hypercholesterolemia not adequately controlled by maximally tolerated statin therapy;

(b) administering every two weeks to the patient 75 mg of an antibody or an antigen-binding fragment thereof which specifically binds human proprotein convertase subtilisin/kexin type 9 (PCSK9);

(c) administering to the patient one or more following doses of 75 mg of the antibody or antigen-binding fragment thereof about every two weeks if, e.g., after 8 weeks, the LDL-C level in the patient is lower than 70 mg/dL, or administering one or more following doses of 150 mg of the antibody or antigen-binding fragment thereof about every two weeks if, e.g., after 8 weeks, the LDL-C level in the patient is greater than or equal to 70 mg/dL, wherein the antibody or antigen-binding fragment thereof comprises an HCVR having the amino acid sequence of SEQ ID NO: 1 and an LCVR having the amino acid sequence of SEQ ID NO: 6, and wherein the patient receives a concomitant insulin therapy.

According to another aspect, the methods comprise a method for treating hypercholesterolemia in a patient with type 1 diabetes mellitus (T1 DM), the method comprising:

(a) selecting a high cardiovascular risk patient receiving insulin therapy that has (i) T1 DM, and (ii) hypercholesterolemia not adequately controlled by maximally tolerated statin therapy;

(b) administering every four weeks to the patient 300 mg of an antibody or an antigen-binding fragment thereof which specifically binds human proprotein convertase subtilisin/kexin type 9 (PCSK9); and

(c) administering to the patient one or more following doses of 300 mg of the antibody or antigen-binding fragment thereof about every four weeks if, after 8 weeks, the LDL-C level in the patient is higher than a threshold level, or administering one or more following doses of 150 mg of the antibody or antigen-binding fragment thereof about every two weeks if, after 8 weeks, the LDL-C level in the patient is lower than or equal to the threshold level,

wherein the antibody or antigen-binding fragment thereof comprises an HCVR having the amino acid sequence of SEQ ID NO: 1 and an LCVR having the amino acid sequence of SEQ ID NO: 6, and wherein the patient receives a concomitant insulin therapy. In one embodiment, the threshold level is 15 mg/dL. In another embodiment, the threshold level is 25 mg/dL.

According to another aspect, the methods comprise a method for treating hypercholesterolemia in a patient with type 2 diabetes mellitus (T2DM), the method comprising:

(a) selecting a high cardiovascular risk patient receiving insulin therapy that has (i) T2DM, and (ii) hypercholesterolemia not adequately controlled by maximally tolerated statin therapy; and
(b) administering to the patient 75 mg, 150 mg, or 300 mg of an antibody or an antigen-binding fragment thereof which specifically binds human proprotein convertase subtilisin/kexin type 9 (PCSK9), wherein the patient receives a concomitant insulin therapy.

In certain embodiments, 75 mg of the antibody or antigen binding fragment is administered to the patient every two weeks. In other embodiments, 150 mg of the antibody or antigen binding fragment is administered to the patient every two weeks. In other embodiments, 300 mg of the antibody or antigen binding fragment is administered to the patient every four weeks.

In certain embodiments, the antibody or antigen-binding fragment thereof comprises the heavy and light chain CDRs of a HCVR/LCVR amino acid sequence pair comprising SEQ ID NOs: 1/6. In certain embodiments, the antibody or antigen-binding fragment thereof comprises the three heavy chain CDRs set forth in SEQ ID NOs: 2, 3, and 4, and the three light chain CDRs set forth in SEQ ID NOs: 7, 8, and 10. In certain embodiments, the antibody or antigen-binding fragment thereof comprises a heavy chain variable region (HCVR) having the amino acid sequence of SEQ ID NO: 1 and a light chain variable region (LCVR) having the amino acid sequence of SEQ ID NO: 6. In certain embodiments, the antibody or antigen-binding fragment thereof competes for binding with an antibody or antigen-binding fragment thereof that comprises an HCVR having the amino acid sequence of SEQ ID NO: 1 and an LCVR having the amino acid sequence of SEQ ID NO: 6. In certain embodiments, the antibody or antigen-binding fragment thereof binds to the same epitope on PCSK9 as an antibody comprising an HCVR having the amino acid sequence of SEQ ID NO: 1 and an LCVR having the amino acid sequence of SEQ ID NO: 6. In certain embodiments, the antibody or antigen-binding fragment thereof binds to an epitope on PCSK9 that overlaps with the epitope of an antibody comprising an HCVR having the amino acid sequence of SEQ ID NO: 1 and an LCVR having the amino acid sequence of SEQ ID NO: 6.

In certain embodiments, the antibody or antigen binding fragment thereof comprises the complementarity determining regions (CDRs) of a heavy chain variable region (HCVR) and a light chain variable region (LCVR) comprising the amino acid sequences set forth in SEQ ID NOs: 85 and 89, respectively. In certain embodiments, the antibody or antigen-binding fragment thereof comprises heavy and light chain CDR amino acid sequences having SEQ ID NOs: 86, 87, 88, 90, 91, and 92. In certain embodiments, the antibody or antigen-binding fragment thereof comprises an HCVR having an amino acid sequence at least 90%, 95%, or 99% identical to the amino acid sequence set forth in SEQ ID NO:85, and an LCVR having an amino acid sequence at least 90%, 95%, or 99% identical to the amino acid sequence set forth in SEQ ID NO:89.

In certain embodiments, the antibody or antigen-binding fragment thereof is selected from the group consisting of alirocumab, evolocumab, bococizumab, lodelcizumab, ralpancizumab and LY3015014. In certain embodiments, the antibody or antigen-binding fragment thereof is alirocumab.

In certain embodiments, the method disclosed herein further comprises (c) administering to the patient one or more following doses of 75 mg of the antibody or antigen-binding fragment thereof about every two weeks if the LDL-C level in the patient is lower than the threshold level, or administering one or more following doses of 150 mg of the antibody or antigen-binding fragment thereof about every two weeks if the LDL-C level in the patient is greater than or equal to the threshold level. In certain embodiments, the threshold level is 70 mg/dL.

In certain embodiments, the method disclosed herein further comprises (c) administering to the patient one or more following doses of 300 mg of the antibody or antigen-binding fragment thereof about every four weeks if, e.g., after 8 weeks, the LDL-C level in the patient is lower than the threshold level, or administering one or more following doses of 150 mg of the antibody or antigen-binding fragment thereof about every two weeks if, e.g., after 8 weeks, the LDL-C level in the patient is greater than or equal to the threshold level. In certain embodiments, the threshold level is 70 mg/dL.

In certain embodiments, the antibody or antigen-binding fragment thereof is administered subcutaneously.

In certain embodiments, the patient further receives a concomitant lipid-modifying therapy (LMT). In certain embodiments, the LMT is selected from the group consisting of a statin, a cholesterol absorption inhibitor, a fibrate, niacin, an omega-3 fatty acid, and a bile acid sequestrant. In certain embodiments, the LMT is a statin therapy. In certain embodiments, the statin is selected from the group consisting of atorvastatin, rosuvastatin, simvastatin, pravastatin, lovastatin, fluvastatin, pitavastatin, and cerivastatin. In certain embodiments, the statin therapy is a maximally tolerated dose statin therapy. In certain embodiments, the cholesterol absorption inhibitor is ezetimibe.

In certain embodiments, the patient is intolerant to a statin.

In certain embodiments, the insulin therapy is selected from the group consisting of human insulin, Insulin glargine, insulin glulisine, insulin detemir, insulin lispro, insulin degludec, insulin aspart, and basal insulin,

In certain embodiments, the patient further receives a concomitant anti-diabetic therapy in addition to insulin therapy. In certain embodiments, the additional anti-diabetic therapy is selected from the group consisting of a glucagon like peptide 1 (GLP-1) therapy, a gastrointestinal peptide, a glucagon receptor agonist or antagonist, a glucose-dependent insulinotropic polypeptide (GIP) receptor agonist or antagonist, a ghrelin antagonist or inverse agonist, xenin, a xenin analogue, a biguanide, a sulfonylurea, a meglitinide, a thiazolidinedione, a DPP-4 inhibitor, an alpha-glucosidase inhibitor, a sodium-dependent glucose transporter 2 (SGLT-2) inhibitor, an SGLT-1 inhibitor, a peroxisome proliferator-activated receptor (PPAR−)(alpha, gamma or alpha/gamma) agonist or modulator, amylin, an amylin analogue, a G-protein coupled receptor 119 (GPR119) agonist, a GPR40 agonist, a GPR120 agonist, a GPR142 agonist, a systemic or low-absorbable TGR5 agonist, a diabetes immunotherapeutic, an anti-inflammatory agents for the treatment of the metabolic syndrome and diabetes, an adenosine monophosphate-activated protein kinase (AMPK) stimulant, an inhibitor of 11-beta-hydroxysteroid dehydrogenase 1, an activator of glucokinase, an inhibitor of diacylglycerol O-acyltransferase (DGAT), a modulator of glucose transporter-4, a somatostatin receptor 3 agonist, a lipid lowering agent, and a combination thereof.

In certain embodiments, the antibody or antigen-binding fragment thereof reduces the LDL-C level of the patient, e.g., by at least 30%, 35%, 40%, or 45%. In certain embodiments, the antibody or antigen-binding fragment thereof reduces the non-HDL-C level of the patient, e.g., by at least 20%, 25%, 30%, or 35%. In certain embodiments, the antibody or antigen-binding fragment thereof reduces the apolipoprotein C3 (ApoC3) level of the patient (e.g., by at least about 6.0%, about 6.5%, about 7.0% or about 7.5% after 12 or 24 weeks of treatment). In certain embodiments, the antibody or antigen-binding fragment thereof reduces the number of lipoprotein particles in the patient (e.g., by at least about 20%, about 30%, about 40% or about 50%% after 12 or 24 weeks of treatment). In other embodiments, the antibody or antigen-binding fragment thereof reduces the size of lipoprotein particles in the patient (e.g., by at least about 1.5%, about 2%, about 2.5%, or about 3% after 12 or 24 weeks of treatment).

In certain embodiments, the antibody or antigen-binding fragment thereof: (a) does not affect the hemoglobin A1c (HbA1c) level of the patient; and/or (b) does not affect the fasting plasma glucose (FPG) level of the patient.

According to another aspect, the methods comprise a method for treating hypercholesterolemia in a patient with type 2 diabetes mellitus (T2DM), the method comprising:

(a) selecting a high cardiovascular risk patient receiving insulin therapy that has (i) T2DM, and (ii) hypercholesterolemia not adequately controlled by maximally tolerated statin therapy;
(b) administering every two weeks to the patient 75 mg of an antibody or an antigen-binding fragment thereof which specifically binds human proprotein convertase subtilisin/kexin type 9 (PCSK9);
(c) administering to the patient one or more following doses of 75 mg of the antibody or antigen-binding fragment thereof about every two weeks if, e.g., after 8 weeks, the LDL-C level in the patient is lower than 70 mg/dL, or administering one or more following doses of 150 mg of the antibody or antigen-binding fragment thereof about every two weeks if, e.g., after 8 weeks, the LDL-C level in the patient is greater than or equal to 70 mg/dL, wherein the antibody or antigen-binding fragment thereof comprises an HCVR having the amino acid sequence of SEQ ID NO: 1 and an LCVR having the amino acid sequence of SEQ ID NO: 6, and wherein the patient receives a concomitant insulin therapy.

According to another aspect, the methods comprise a method for treating hypercholesterolemia in a patient with type 2 diabetes mellitus (T2DM), the method comprising:

(a) selecting a high cardiovascular risk patient receiving insulin therapy that has (i) T2DM, and (ii) hypercholesterolemia not adequately controlled by maximally tolerated statin therapy;

(b) administering every four weeks to the patient 300 mg of an antibody or an antigen-binding fragment thereof which specifically binds human proprotein convertase subtilisin/kexin type 9 (PCSK9); and

(c) administering to the patient one or more following doses of 300 mg of the antibody or antigen-binding fragment thereof about every four weeks if, after 8 weeks, the LDL-C level in the patient is higher than a threshold level, or administering one or more following doses of 150 mg of the antibody or antigen-binding fragment thereof about every two weeks if, after 8 weeks, the LDL-C level in the patient is lower than or equal to the threshold level,

wherein the antibody or antigen-binding fragment thereof comprises an HCVR having the amino acid sequence of SEQ ID NO: 1 and an LCVR having the amino acid sequence of SEQ ID NO: 6, and wherein the patient receives a concomitant insulin therapy. In one embodiment, the threshold level is 15 mg/dL. In another embodiment, the threshold level is 25 mg/dL.

According to another aspect, the methods comprise a method for treating hypercholesterolemia in a patient with T2DM and atherosclerotic cardiovascular disease (ASCVD), the method comprising:

(a) selecting a high cardiovascular risk patient receiving insulin therapy that has (i) T2DM, (ii) ASCVD, and (iii) hypercholesterolemia not adequately controlled by maximally tolerated statin therapy; and
(b) administering to the patient 75 mg, 150 mg or 300 mg of an antibody or an antigen-binding fragment thereof which specifically binds human proprotein convertase subtilisin/kexin type 9 (PCSK9), wherein the patient receives a concomitant insulin therapy.

In certain embodiments, the ASCVD is defined as coronary heart disease (CHD), ischemic stroke, or peripheral arterial disease. In certain embodiments, the CHD comprises acute myocardial infarction, silent myocardial infarction, and unstable angina.

In certain embodiments, the 75 mg of the antibody or antigen binding fragment is administered to the patient every two weeks. In certain embodiments, the 150 mg of the antibody or antigen binding fragment is administered to the patient every two weeks. In certain embodiments, the 300 mg of the antibody or antigen binding fragment is administered to the patient every four weeks.

In certain embodiments, the antibody or antigen-binding fragment thereof comprises the heavy and light chain CDRs of a HCVR/LCVR amino acid sequence pair comprising SEQ ID NOs: 1/6. In certain embodiments, the antibody or antigen-binding fragment thereof comprises the three heavy chain CDRs set forth in SEQ ID NOs: 2, 3, and 4, and the three light chain CDRs set forth in SEQ ID NOs: 7, 8, and 10. In certain embodiments, the antibody or antigen-binding fragment thereof comprises a heavy chain variable region (HCVR) having the amino acid sequence of SEQ ID NO: 1 and a light chain variable region (LCVR) having the amino acid sequence of SEQ ID NO: 6. In certain embodiments, the antibody or antigen-binding fragment thereof binds to the same epitope on PCSK9 as an antibody comprising an HCVR having the amino acid sequence of SEQ ID NO: 1 and an LCVR having the amino acid sequence of SEQ ID NO: 6. In certain embodiments, the antibody or antigen-binding fragment thereof binds to an epitope on PCSK9 that overlaps with the epitope of an antibody comprising an HCVR having the amino acid sequence of SEQ ID NO: 1 and an LCVR having the amino acid sequence of SEQ ID NO: 6.

In certain embodiments, the antibody or antigen binding fragment thereof comprises the complementarity determining regions (CDRs) of a heavy chain variable region (HCVR) and a light chain variable region (LCVR) comprising the amino acid sequences set forth in SEQ ID NOs: 85 and 89, respectively. In certain embodiments, the antibody or antigen-binding fragment thereof comprises heavy and light chain CDR amino acid sequences having SEQ ID NOs: 86, 87, 88, 90, 91, and 92. In certain embodiments, the antibody or antigen-binding fragment thereof comprises an HCVR having an amino acid sequence at least 90%, 95%, or 99% identical to the amino acid sequence set forth in SEQ ID NO:85, and an LCVR having an amino acid sequence at least 90%, 95%, or 99% identical to the amino acid sequence set forth in SEQ ID NO:89.

In certain embodiments, the antibody or antigen-binding fragment thereof is selected from the group consisting of alirocumab, evolocumab, bococizumab, lodelcizumab, ralpancizumab, and LY3015014. In certain embodiments, the antibody or antigen-binding fragment thereof is alirocumab.

In certain embodiments, the method further comprises:

(c) administering to the patient one or more following doses of 75 mg of the antibody or antigen-binding fragment thereof about every two weeks if the LDL-C level in the patient is lower than the threshold level, or administering one or more following doses of 150 mg of the antibody or antigen-binding fragment thereof about every two weeks if the LDL-C level in the patient is greater than or equal to the threshold level.

In certain embodiments, the method further comprises:

(c) administering to the patient one or more following doses of 300 mg of the antibody or antigen-binding fragment thereof about every four weeks if the LDL-C level in the patient is lower than a threshold level, or administering one or more following doses of 150 mg of the antibody or antigen-binding fragment thereof about every two weeks if the LDL-C level in the patient is greater than or equal to the threshold level.

In certain embodiments, the threshold level is 70 mg/dL.

In certain embodiments, the antibody or antigen-binding fragment thereof is administered subcutaneously.

In certain embodiments, the patient further receives a concomitant lipid-modifying therapy (LMT). In certain embodiments, the LMT is selected from the group consisting of a statin, a cholesterol absorption inhibitor, a fibrate, niacin, an omega-3 fatty acid, and a bile acid sequestrant. In certain embodiments, the LMT is a statin therapy. In certain embodiments, the statin is selected from the group consisting of atorvastatin, rosuvastatin, simvastatin, pravastatin, lovastatin, fluvastatin, pitavastatin, and cerivastatin. In certain embodiments, the statin therapy is a maximally tolerated dose statin therapy. In certain embodiments, the cholesterol absorption inhibitor is ezetimibe.

In certain embodiments, the patient is intolerant to a statin.

In certain embodiments, the insulin therapy is selected from the group consisting of human insulin, Insulin glargine, insulin glulisine, insulin detemir, insulin lispro, insulin degludec, insulin aspart, and basal insulin. In certain embodiments, the patient receives a concomitant anti-diabetic therapy in addition to insulin therapy. In certain embodiments, the additional anti-diabetic therapy is selected from the group consisting of an a glucagon like peptide 1 (GLP-1) therapy, a gastrointestinal peptide, a glucagon receptor agonist or antagonist, a glucose-dependent insulinotropic polypeptide (GIP) receptor agonist or antagonist, a ghrelin antagonist or inverse agonist, xenin, a xenin analogue, a biguanide, a sulfonylurea, a meglitinide, a thiazolidinedione, a DPP-4 inhibitor, an alpha-glucosidase inhibitor, a sodium-dependent glucose transporter 2 (SGLT-2) inhibitor, an SGLT-1 inhibitor, a peroxisome proliferator-activated receptor (PPAR−)(alpha, gamma or alpha/gamma) agonist or modulator, amylin, an amylin analogue, a G-protein coupled receptor 119 (GPR119) agonist, a GPR40 agonist, a GPR120 agonist, a GPR142 agonist, a systemic or low-absorbable TGR5 agonist, a diabetes immunotherapeutic, an anti-inflammatory agents for the treatment of the metabolic syndrome and diabetes, an adenosine monophosphate-activated protein kinase (AMPK) stimulant, an inhibitor of 11-beta-hydroxysteroid dehydrogenase 1, an activator of glucokinase, an inhibitor of diacylglycerol O-acyltransferase (DGAT), a modulator of glucose transporter-4, a somatostatin receptor 3 agonist, a lipid lowering agent, and a combination thereof.

In certain embodiments, the antibody or antigen-binding fragment thereof reduces the LDL-C level of the patient, e.g., by at least 30%, 35%, 40%, or 45%. In certain embodiments, the antibody or antigen-binding fragment thereof reduces the non-HDL-C level of the patient, e.g., by at least 20%, 25%, 30%, 35%. In certain embodiments, the antibody or antigen-binding fragment thereof reduces the ApoC3 level of the patient. In certain embodiments, the antibody or antigen-binding fragment thereof reduces the number and/or size of lipoprotein particles in the patient. In certain embodiments, the antibody or antigen-binding fragment thereof:

(a) does not affect the hemoglobin A1c (HbA1c) level of the patient; and/or
(b) does not affect the fasting plasma glucose (FPG) level of the patient.

According to another aspect, the methods comprise a method for treating hypercholesterolemia in a patient with T2DM and ASCVD, the method comprising:

(a) selecting a high cardiovascular risk patient receiving insulin therapy that has (i) T2DM, (ii) ASCVD, and (iii) hypercholesterolemia not adequately controlled by maximally tolerated statin therapy;
(b) administering every two weeks to the patient 75 mg of an antibody or an antigen-binding fragment thereof which specifically binds human proprotein convertase subtilisin/kexin type 9 (PCSK9); and
(c) administering to the patient one or more following doses of 75 mg of the antibody or antigen-binding fragment thereof about every two weeks if the LDL-C level in the patient is lower than 70 mg/dL, or administering one or more following doses of 150 mg of the antibody or antigen-binding fragment thereof about every two weeks if the LDL-C level in the patient is greater than or equal to 70 mg/dL,
wherein the antibody or antigen-binding fragment thereof comprises an HCVR having the amino acid sequence of SEQ ID NO: 1 and an LCVR having the amino acid sequence of SEQ ID NO: 6, and wherein the patient receives a concomitant insulin therapy.

Other embodiments will become apparent from a review of the ensuing detailed description.

### BRIEF DESCRIPTION OF THE FIGURES

FIG. 1 is a diagram illustrating the overall design of the main phase of the study described in Example 2 herein. The study includes a screening period, a double-blinded treatment period, and a safety observation period.

FIG. 2 is a graph showing the LS means (+/−SE) of percent change of calculated LDL-C levels from baseline in the ITT population of patients with Type 1 Diabetes as per IVRS. Least-squares (LS) means and standard errors (SE) were taken from mixed-effect model with repeated measures (MMRM) analysis. The model includes the fixed categorical effects of treatment group, randomization strata as per IVRS, time point, and the interactions treatment group-by-time point, strata-by-time point, treatment group-by-strata and treatment group-by-strata-by-time point, as well as the continuous fixed covariates of baseline calculated LDL-C value and baseline-by-time point interaction. MMRM model was run on all patients in the ITT population (i.e. Type 1 and Type 2 Diabetes patients).

FIG. 3 is a graph showing the LS means (+/−SE) of percent change of calculated LDL-C levels from baseline in the ITT population of patients with Type 2 Diabetes as per IVRS. Least-squares (LS) means and standard errors (SE) were taken from mixed-effect model with repeated measures (MMRM) analysis. The model includes the fixed categorical effects of treatment group, randomization strata as per IVRS, time point, and the interactions treatment group-by-time point, strata-by-time point, treatment group-by-strata and treatment group-by-strata-by-time point, as well as the continuous fixed covariates of baseline calculated LDL-C value and baseline-by-time point interaction. MMRM model was run on all patients in the ITT population (i.e. Type 1 and Type 2 Diabetes patients).

FIG. 4 is a graph showing the percentage changes from baseline to Week 24 in non-HDL-C, LDL-C, ApoB, and LDL-PN in the ITT population with Type 2 Diabetes and ASCVD.

FIG. 5 is a graph showing the percentages of individuals achieving non-HDL-C<100 mg/dL, LDL-C<70 mg/dL, and ApoB<80 mg/dL at Week 24 in the ITT population with Type 2 Diabetes and ASCVD.

### DETAILED DESCRIPTION

The methods are not limited to particular methods and experimental conditions described, as such methods and conditions may vary. It is also to be understood that the terminology used herein is for the purpose of describing particular embodiments only, and is not intended to be limiting, since the scope of the present methods will be limited only by the appended claims.

Unless defined otherwise, all technical and scientific terms used herein have the same meaning as commonly understood by one of ordinary skill in the art. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used, the preferred methods and materials are now described. All publications mentioned herein are incorporated herein by reference to describe in their entirety.

Methods for Treating a Patient with Hypercholesterolemia and Diabetes on Insulin Therapy

Provided are methods and compositions for treating diabetic patients with hypercholesterolemia on insulin therapy. According to certain embodiments, these methods result in a reduction of lipoprotein levels (e.g., LDL-C and/or Lp(a)) in the serum of such patients.

The present disclosure also provides a PCSK9 inhibitor (e.g., an antibody or antigen-binding fragment thereof that specifically binds PCSK9 (e.g., human PCSK9) or a composition comprising the PCSK9 inhibitor for use in treating a diabetic patients with hypercholesterolemia on insulin therapy. In certain embodiments, the PCSK9 inhibitor or composition is useful in reducing the levels of lipoprotein (e.g., LDL-C and/or Lp(a)) in the serum of such patients.

As used herein, the term “lipoprotein” means a biomolecular particle containing both protein and lipid. Examples of lipoproteins include, e.g., low density lipoprotein (LDL), very low density lipoprotein (VLDL), intermediate density lipoprotein (IDL), and lipoprotein (a) (Lp(a)).

Diabetes mellitus, often simply called diabetes, is a group of metabolic diseases in which a person has high blood sugar levels, either because the body does not produce enough insulin, or because cells do not respond to the insulin that is produced. The most common types of diabetes are: (1) type 1 diabetes, where the body fails to produce insulin; (2) type 2 diabetes, where the body fails to use insulin properly, combined with an increase in insulin deficiency over time; and (3) gestational diabetes, where women develop diabetes due to their pregnancy. All forms of diabetes increase the risk of long-term complications, which typically develop after many years. Most of these long-term complications are based on damage to blood vessels and can be divided into the two categories “macrovascular” disease, arising from atherosclerosis of larger blood vessels and “microvascular” disease, arising from damage of small blood vessels. Examples for macrovascular disease conditions are ischemic heart disease, myocardial infarction, stroke and peripheral vascular disease. Examples for microvascular diseases are diabetic retinopathy, diabetic nephropathy, as well as diabetic neuropathy.

According to certain embodiments, the patient to be treated has type 1 diabetes mellitus (T1 DM) or type 2 diabetes mellitus (T2DM) and is receiving insulin therapy. In certain embodiments, the patient has been diagnosed with T1 DM or T2DM for at least one year. In certain embodiments, the patient was diagnosed with T1 DM prior to the age of 30 years. In certain embodiments, the T1 DM patient has a C-peptide level lower than 0.2 pmol/mL. In certain embodiments, the patient has a glycosylated hemoglobin (HbA1c) level lower than 10%.

According to certain embodiments, the patient to be treated has hypercholesterolemia not adequately controlled by a lipid modifying therapy (LMT). Hypercholesterolemia is considered not to be adequately controlled by an LMT if serum LDL-C concentrations in patient are not reduced to a recognized, medically-acceptable level, e.g., less than 70 mg/dL, (taking into account the patient's relative risk of coronary heart disease) after at least 4 weeks on the LMT. In certain embodiments, the LMT is a maximally tolerated statin therapy. As used herein, “maximally tolerated statin therapy” means the highest dose of statin that can be administered to a patient without causing unacceptable adverse side effects in that patient. For example, the methods disclosed herein include treating a patient with T1 DM or T2DM who has hypercholesterolemia that is not adequately controlled by a daily dose of a statin selected from the group consisting of atorvastatin (including atorvastatin+ezetimibe), rosuvastatin, cerivastatin, pitavastatin, fluvastatin, lovastatin, simvastatin (including simvastatin+ezetimibe), pravastatin, and combinations thereof. In certain embodiments, a patient does not receive a concomitant statin therapy if the patient is intolerant to this therapy. Statin intolerant patients may have, for example, skeletal muscle-related symptoms, other than those due to strain or trauma, such as pain, aches, weakness, or cramping, that begin or increase during statin therapy and stopped when statin therapy is discontinued.

### Patient Selection

The present methods and compositions are useful for treating patients who have hypercholesterolemia and diabetes and are receiving insulin therapy. The patient to be treated may also exhibit one or more of additional selection criteria. For example, a patient may be selected for treatment if the patient has a calculated LDL-C level greater than or equal to 70 mg/dL, 100 mg/dL, or 130 mg/dL. The patient may be treated with an maximally tolerated dose of statin, optionally in combination with at least one other lipid modifying therapy (LMT) for at least 4 weeks, or wherein the patient has been treated with optimal dose of at least one non-statin LMT for at least 4 weeks if the patient is statin intolerant. The maximally tolerated dose of statin can be defined, for example, as the dose prescribed based on regional practice or local guidelines or is the dose that is maximally tolerated due to adverse effects on higher doses as specified in the local prescribing information for pediatric patients. Statin intolerance can be defined, for example, as inability to tolerate at least 2 statins: one statin at the lowest daily starting dose, and another statin at any dose, due to skeletal muscle-related symptoms, other than those due to strain or trauma, such as pain, aches, weakness, or cramping, that began or increased during statin therapy and stopped when statin therapy was discontinued. Patients not receiving a daily regimen of a statin (e.g., 1 to 3 times weekly) are also considered as not able to tolerate a daily dose.

Additionally, a patient may be selected for treatment if the patient has a high cardiovascular (CV) risk. In certain embodiments, the high CV risk patient has a documented history of cardiovascular disease (CVD) and/or at least one additional CV risk factor. CVD includes without limitation coronary heart disease (CHD) and CHD risk equivalents. CHD includes without limitation acute myocardial infarction (MI), silent MI, unstable angina, coronary revascularization procedure (e.g., percutaneuous coronary intervention (PCI) or coronary artery bypass graft surgery (CABG)), and clinically significant CHD (e.g., diagnosed by invasive or noninvasive testing, such as coronary angiography, stress test using treadmill, stress echocardiography, or nuclear imaging). CHD risk equivalents include without limitation peripheral arterial disease (e.g., as described in the inclusion criteria of Example 2) and previous ischemic stroke with a focal ischemic neurological deficit that persisted more than 24 hours, of atherothrombotic origin. CV risk factors include without limitation hypertension, current cigarette smoking, age 45 years for men and 55 years for women, history of micro/macroalbuminuria, history of diabetic retinopathy, family history of premature CHD (in father or brother before 55 years of age; in mother or sister before 65 years of age), low HDL-C (male <40 mg/dL [1.0 mmol/L] and female <50 mg/dL [1.3 mmol/L]), and documented chronic kidney disease (CKD) (e.g., as defined in the inclusion criteria of Example 2).

In certain embodiments, the high CV risk patient has atherosclerotic cardiovascular disease (ASCVD). In certain embodiments, ASCVD is defined as coronary heart disease (CHD), ischemic stroke, or peripheral arterial disease. In certain embodiments, the CHD comprises acute myocardial infarction, silent myocardial infarction, and unstable angina. In certain embodiments, the CHD is defined as acute myocardial infarction, silent myocardial infarction, or unstable angina.

### Insulin Therapy

As indicated herein, diabetic patients selected for treatment with the methods of the invention have received, and continue to receive, insulin therapy comprising insulin or its derivatives. Insulins on the market differ in the origin of the insulin (e.g., bovine, porcine, human insulin) and also in their composition, whereby the profile of action (onset of action and duration of action) can be influenced. By combining different insulin products it is possible to obtain a wide variety of profiles of action and to set blood sugar levels which are as close as possible to physiological. Exemplary insulin therapies may include naturally-occurring insulin, such as human insulin, as well as modified insulins with an extended duration of action, such as insulin glargine (Gly(A21)-Arg(B31)-Arg(B32) human insulin, e.g., Lantue). Insulin glargine is injected as an acidic, clear solution and, on account of its solution properties in the physiological pH range of the subcutaneous tissue, is precipitated as a stable hexamer associate. Insulin glargine is injected once daily and is notable over other long-activity insulins for its flat serum profile and the associated reduction in the risk of nocturnal hypoglycemias (Schubert-Zsilavecz et al., 2:125-130 (2001)). Insulin glargine may be administered at concentrations higher than 100 U/mL, e.g. 270-330 U/mL of insulin glargine or 300 U/mL of insulin glargine (as disclosed in EP 2387989), Other exemplary insulin therapies include: insulin glulisine (e.g. Apidra®, insulin detemir (e.g. Levemir®, insulin lispro (e.g. Humalog®, Liprolog®), insulin degludec (e.g. DegludecPlus®, IdegLira (NN9068)), insulin aspart and aspart formulations (e.g. NovoLog®), basal insulin and analogues (e.g. LY2605541, LY2963016, NN1436), PEGylated insulin lispro (e.g. LY-275585), long-acting insulins (e.g. NN1436, Insumera (PE0139), AB-101, AB-102, Sensulin LLC), intermediate-acting insulins (e.g. Humulin®N, Novolin®N), fast-acting and short-acting insulins (e.g. Humulin®R, Novolin®R, Linjeta® (VIAject®), PH20 insulin, NN1218, HinsBet®), premixed insulins, SuliXen®, NN1045, insulin plus Symlin®, PE-0139, ACP-002 hydrogel insulin, and oral, inhalable, transdermal and buccal or sublingual insulins (e.g. Exubera®, Nasulin®, Afrezza®, insulin tregopil, TPM-02 insulin, Capsulin®, Oral-lyn®, Cobalamin® oral insulin, ORMD-0801, Oshadi oral insulin, NN1953, NN1954, NN1956, VIAtab®). Also suitable are those insulin derivatives which are bonded to albumin or another protein by a bifunctional linker.

### PCSK9 Inhibitors

The methods comprise administering to a patient a therapeutic composition comprising a PCSK9 inhibitor. As used herein, a “PCSK9 inhibitor” is any agent which binds to or interacts with human PCSK9 and inhibits the normal biological function of PCSK9 in vitro or in vivo. Non-limiting examples of categories of PCSK9 inhibitors include small molecule PCSK9 antagonists, nucleic acid-based inhibitors of PCSK9 expression or activity (e.g., siRNA or antisense), peptide-based molecules that specifically interact with PCSK9 (e.g., peptibodies), receptor molecules that specifically interact with PCSK9, proteins comprising a ligand-binding portion of an LDL receptor, PCSK9-binding scaffold molecules (e.g., DARPins, HEAT repeat proteins, ARM repeat proteins, tetratricopeptide repeat proteins, fibronectin-based scaffold constructs, and other scaffolds based on naturally occurring repeat proteins, etc., [see, e.g., Boersma and Pluckthun, 2011, Curr. Opin. Biotechnol. 22:849-857, and references cited therein]), and anti-PCSK9 aptamers or portions thereof. According to certain embodiments, PCSK9 inhibitors that can be used in the context of the present methods are anti-PCSK9 antibodies or antigen-binding fragments of antibodies that specifically bind human PCSK9.

The term “human proprotein convertase subtilisin/kexin type 9” or “human PCSK9” or “hPCSK9”, as used herein, refers to PCSK9 having the nucleic acid sequence shown in SEQ ID NO:197 and the amino acid sequence of SEQ ID NO:198, or a biologically active fragment thereof.

The term “antibody”, as used herein, is intended to refer to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V₁) and a light chain constant region. The light chain constant region comprises one domain (CO). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments, the FRs of the anti-PCSK9 antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody include: (i) V_{H}-C_{H}1; (ii) V_{H}-C_{H}2; (iii) V_{H}-C_{H}3; (iv) V_{H}—C_{H}1-C_{H}2; (V) V_{H}-C_{H}1-C_{H}2-C_{H}3, (vi) V_{H}-C_{H}2-C_{H}3; (vii) V_{H}-C_{L}; V_{L}—C_{H}1; (ix) V_{L}-C_{H}2; (v) V_{L}- C_{H}3; (xi) V_{L}-C_{H}1-C_{H}2; (xii) V_{L}-C_{H}1-C_{H}2-C_{H}3, (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present methods using routine techniques available in the art.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody”, as used herein, is intended to include antibodies having variable and constant regions derived from human germline immunoglobulin sequences. The human antibodies may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant methods encompass antibodies having one or more mutations in the hinge, C_{H}2 or C_{H}3 region which may be desirable, for example, in production, to improve the yield of the desired antibody form.

An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present methods. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” PCSK9, as used in the context of the present methods, includes antibodies that bind PCSK9 or portion thereof with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human PCSK9, however, has cross-reactivity to other antigens, such as PCSK9 molecules from other (non-human) species.

The anti-PCSK9 antibodies useful for the present methods may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The methods include the use of antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the present methods.

The methods include the use of anti-PCSK9 antibodies comprising variants of any of the HCVR, LCVR, and/or CDR amino acid sequences disclosed herein having one or more conservative substitutions. For example, the present methods include the use of anti-PCSK9 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “surface plasmon resonance”, as used herein, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, N.J.).

The term “K_{D}”, as used herein, is intended to refer to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

According to certain embodiments, the anti-PCSK9 antibody used in the methods is an antibody with pH-dependent binding characteristics. As used herein, the expression “pH-dependent binding” means that the antibody or antigen-binding fragment thereof exhibits “reduced binding to PCSK9 at acidic pH as compared to neutral pH” (for purposes of the present disclosure, both expressions may be used interchangeably). For the example, “antibodies with pH-dependent binding characteristics” includes antibodies and antigen-binding fragments thereof that bind PCSK9 with higher affinity at neutral pH than at acidic pH. In certain embodiments, the antibodies and antigen-binding fragments bind PCSK9 with at least 3, 5, 10, 15, 20, 25, 30, 35, 40, 45, 50, 55, 60, 65, 70, 75, 80, 85, 90, 95, 100, or more times higher affinity at neutral pH than at acidic pH.

According to this aspect, the anti-PCSK9 antibodies with pH-dependent binding characteristics may possess one or more amino acid variations relative to the parental anti-PCSK9 antibody. For example, an anti-PCSK9 antibody with pH-dependent binding characteristics may contain one or more histidine substitutions or insertions, e.g., in one or more CDRs of a parental anti-PCSK9 antibody. Thus, according to certain embodiments, methods are provided comprising administering an anti-PCSK9 antibody which comprises CDR amino acid sequences (e.g., heavy and light chain CDRs) which are identical to the CDR amino acid sequences of a parental anti-PCSK9 antibody, except for the substitution of one or more amino acids of one or more CDRs of the parental antibody with a histidine residue. The anti-PCSK9 antibodies with pH-dependent binding may possess, e.g., 1, 2, 3, 4, 5, 6, 7, 8, 9, or more histidine substitutions, either within a single CDR of a parental antibody or distributed throughout multiple (e.g., 2, 3, 4, 5, or 6) CDRs of a parental anti-PCSK9 antibody. For example, the present methods include the use of anti-PCSK9 antibodies with pH-dependent binding comprising one or more histidine substitutions in HCDR1, one or more histidine substitutions in HCDR2, one or more histidine substitutions in HCDR3, one or more histidine substitutions in LCDR1, one or more histidine substitutions in LCDR2, and/or one or more histidine substitutions in LCDR3, of a parental anti-PCSK9 antibody.

As used herein, the expression “acidic pH” means a pH of 6.0 or less (e.g., less than about 6.0, less than about 5.5, less than about 5.0, etc.). The expression “acidic pH” includes pH values of about 6.0, 5.95, 5.90, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

Non-limiting examples of anti-PCSK9 antibodies that can be used in the context of the present methods include, e.g., alirocumab, evolocumab, bococizumab, lodelcizumab, ralpancizumab, LY3015014, or antigen-binding portions of any of the foregoing antibodies.

### Preparation of Human Antibodies

Methods for generating human antibodies in transgenic mice are known in the art. Any such known methods can be used in the context of the present methods to make human antibodies that specifically bind to human PCSK9.

Using VELOCIMMUNE™ technology (see, for example, U.S. Pat. No. 6,596,541, Regeneron Pharmaceuticals) or any other known method for generating monoclonal antibodies, high affinity chimeric antibodies to PCSK9 are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc, using standard procedures known to those skilled in the art. The mouse constant regions are replaced with a desired human constant region to generate the fully human antibody, for example wild-type or modified IgG1 or IgG4. While the constant region selected may vary according to specific use, high affinity antigen-binding and target specificity characteristics reside in the variable region.

In general, the antibodies that can be used possess high affinities, as described above, when measured by binding to antigen either immobilized on solid phase or in solution phase. The mouse constant regions are replaced with desired human constant regions to generate the fully human antibodies. While the constant region selected may vary according to specific use, high affinity antigen-binding and target specificity characteristics reside in the variable region.

Specific examples of human antibodies or antigen-binding fragments of antibodies that specifically bind PCSK9 which can be used in the context of the methods include any antibody or antigen-binding fragment which comprises the three heavy chain CDRs (HCDR1, HCDR2 and HCDR3) contained within a heavy chain variable region (HCVR) having an amino acid sequence selected from the group consisting of SEQ ID NOs: 1 and 11, or a substantially similar sequence thereof having at least 90%, at least 95%, at least 98% or at least 99% sequence identity. Alternatively, specific examples of human antibodies or antigen-binding fragments of antibodies that specifically bind PCSK9 which can be used in the context of the methods include any antibody or antigen-binding fragment which comprises the three heavy chain CDRs (HCDR1, HCDR2 and HCDR3) contained within a heavy chain variable region (HCVR) having an amino acid sequence selected from the group consisting of SEQ ID NOs 37, 45, 53, 61, 69, 77, 85, 93, 101, 109, 117, 125, 133, 141, 149, 157, 165, 173, 181, and 189, or a substantially similar sequence thereof having at least 90%, at least 95%, at least 98% or at least 99% sequence identity. The antibody or antigen-binding fragment may comprise the three light chain CDRs (LCVR1, LCVR2, LCVR3) contained within a light chain variable region (LCVR) having an amino acid sequence selected from the group consisting of SEQ ID NOs 6 and 15, or a substantially similar sequence thereof having at least 90%, at least 95%, at least 98% or at least 99% sequence identity. Alternatively, the antibody or antigen-binding fragment may comprise the three light chain CDRs (LCVR1, LCVR2, LCVR3) contained within a light chain variable region (LCVR) having an amino acid sequence selected from the group consisting of SEQ ID NOs 41, 49, 57, 65, 73, 81, 89, 97, 105, 113, 121, 129, 137, 145, 153, 161, 169, 177, 185, and 193, or a substantially similar sequence thereof having at least 90%, at least 95%, at least 98% or at least 99% sequence identity.

Sequence identity between two amino acids sequences is determined over the entire length of the reference amino acid sequence, i.e. the amino acid sequence identified with a SEQ ID NO, using the best sequence alignment and/or over the region of the best sequence alignment between the two amino acid sequences, wherein the best sequence alignment can be obtained with art known tools, e.g. Align, using standard settings, preferably EMBOSS::needle, Matrix: Blosum62, Gap Open 10.0, Gap Extend 0.5.

In certain embodiments, the antibody or antigen-binding protein comprises the six CDRs (HCDR1, HCDR2, HCDR3, LCDR1, LCDR2 and LCDR3) from the heavy and light chain variable region amino acid sequence pairs (HCVR/LCVR) selected from the group consisting of SEQ ID NOs: 1/6 and 11/15. Alternatively, in certain embodiments, the antibody or antigen-binding protein comprises the six CDRs (HCDR1, HCDR2, HCDR3, LCDR1, LCDR2 and LCDR3) from the heavy and light chain variable region amino acid sequence pairs (HCVR/LCVR) selected from the group consisting of SEQ ID NOs: 37/41, 45/49, 53/57, 61/65, 69/73, 77/81, 85/89, 93/97, 101/105, 109/113, 117/121, 125/129, 133/137, 141/145, 149/153, 157/161, 165/169, 173/177, 181/185, and 189/193.

In certain embodiments, the anti-PCSK9 antibody, or antigen-binding protein, that can be used in the methods has HCDR1/HCDR2/HCDR3/LCDR1/LCDR2/LCDR3 amino acid sequences selected from SEQ ID NOs: 2/3/4/7/8/10 (mAb316P [also referred to as “REGN727,” or “alirocumab”]) and 12/13/14/16/17/18 (mAb300N) (See US Patent App. Publ No. 2010/0166768) and 12/13/14/16/17/18, wherein SEQ ID NO:16 comprises a substitution of histidine for leucine at amino acid residue 30 (L30H).

In certain embodiments, the antibody or antigen-binding protein comprises HCVR/LCVR amino acid sequence pairs selected from the group consisting of SEQ ID NOs: 1/6 and 11/15. In certain exemplary embodiments, the antibody or antigen-binding protein comprises an HCVR amino acid sequence of SEQ ID NO: 1 and an LCVR amino acid sequence of SEQ ID NO: 6. In certain exemplary embodiments, the antibody or antigen-binding protein comprises an HCVR amino acid sequence of SEQ ID NO: 11 and an LCVR amino acid sequence of SEQ ID NO: 15. In certain exemplary embodiments, the antibody or antigen-binding protein comprises an HCVR amino acid sequence of SEQ ID NO: 11 and an LCVR amino acid sequence of SEQ ID NO: 15 comprising a substitution of histidine for leucine at amino acid residue 30 (L30H).

### Pharmaceutical Compositions and Methods of Administration

The present methods include administering a PCSK9 inhibitor to a patient, wherein the PCSK9 inhibitor is contained within a pharmaceutical composition. The pharmaceutical compositions are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Exemplary pharmaceutical formulations comprising anti-PCSK9 antibodies that can be used in the context of the present methods include any of the formulations as set forth in U.S. Pat. No. 8,795,669 (describing, inter alia, exemplary formulations comprising alirocumab), or in WO2013/166448, or WO2012/168491.

Various delivery systems are known and can be used to administer the pharmaceutical composition, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition can be delivered subcutaneously or intravenously with a standard needle and syringe. In addition, with respect to subcutaneous delivery, a pen delivery device readily has applications in delivering a pharmaceutical composition. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™ OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present methods include, but are not limited to the SOLOSTAR™ pen (sanofi-aventis), the FLEXPEN™ (Novo Nordisk), and the KW IKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park Ill.), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Dosage

The amount of PCSK9 inhibitor (e.g., anti-PCSK9 antibody) administered to a patient is, generally, a therapeutically effective amount. As used herein, the phrase “therapeutically effective amount” means a dose of PCSK9 inhibitor that results in a detectable reduction (at least about 5%, 10%, 15%, 20%, 25%, 30%, 35%, 40%, 45%, 50%, 55%, 60%, 65%, 70%, 75%, or more from baseline) in one or more parameters selected from the group consisting of LDL-C, ApoB, ApoB100, non-HDL-C, total cholesterol, VLDL-C, triglycerides, ApoC3, TRL particles, Lp(a) and remnant cholesterol).

In the case of an anti-PCSK9 antibody, a therapeutically effective amount can be from about 0.05 mg to about 600 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 75 mg, about 80 mg, about 90 mg, about 100 mg, about 110 mg, about 120 mg, about 130 mg, about 140 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-PCSK9 antibody. According to certain exemplary embodiments, a therapeutically effective amount of an anti-PCSK9 antibody is 30 mg, 40 mg or 75 mg (e.g., in the case of alirocumab for patients with body weight less than 50 kg, and/or younger than or equal to 17 years old), 50 mg, 75 mg or 150 mg (e.g., in the case of alirocumab for patients with body weight greater than or equal to 50 kg, and/or younger than or equal to 17 years old), or 140 mg or 420 mg (e.g., in the case of evolocumab). Other dosing amounts of PCSK9 inhibitors will be apparent to persons of ordinary skill in the art.

The amount of anti-PCSK9 antibody contained within the individual doses may be expressed in terms of milligrams of antibody per kilogram of patient body weight (i.e., mg/kg). For example, the anti-PCSK9 antibody may be administered to a patient at a dose of about 0.0001 to about 10 mg/kg of body weight.

### Administration Regimens

According to certain embodiments, multiple doses of a PCSK9 inhibitor (i.e., a pharmaceutical composition comprising a PCSK9 inhibitor) may be administered to a subject over a defined time course (e.g., on top of a daily therapeutic statin regimen or other background LMT). The methods according to this aspect comprise sequentially administering to a subject multiple doses of a PCSK9 inhibitor. As used herein, “sequentially administering” means that each dose of PCSK9 inhibitor is administered to the subject at a different point in time, e.g., on different days separated by a predetermined interval (e.g., hours, days, weeks or months). The present methods includes sequentially administering to the patient a single initial dose of a PCSK9 inhibitor, followed by one or more secondary doses of the PCSK9 inhibitor, and optionally followed by one or more tertiary doses of the PCSK9 inhibitor.

The terms “initial dose,” “secondary doses,” and “tertiary doses,” refer to the temporal sequence of administration of the individual doses of a pharmaceutical composition comprising a PCSK9 inhibitor. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the PCSK9 inhibitor, but generally may differ from one another in terms of frequency of administration. In certain embodiments, however, the amount of PCSK9 inhibitor contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

According to exemplary embodiments, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of antigen-binding molecule which is administered to a patient prior to the administration of the very next dose in the sequence with no intervening doses.

The methods according to this aspect may comprise administering to a patient any number of secondary and/or tertiary doses of a PCSK9 inhibitor. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2, 4, 6, 8 or more weeks after the immediately preceding dose. Similarly, in embodiments involving multiple tertiary doses, each tertiary dose may be administered at the same frequency as the other tertiary doses. For example, each tertiary dose may be administered to the patient 1 to 2, 4, 6, 8 or more weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

The present methods include administration regimens comprising an up-titration option (also referred to herein as “dose modification”). As used herein, an “up-titration option” means that, after receiving a particular number of doses of a PCSK9 inhibitor, if a patient has not achieved a specified reduction in one or more defined therapeutic parameters, the dose of the PCSK9 inhibitor is thereafter increased. For example, in the case of a therapeutic regimen comprising administration of 75 mg doses of an anti-PCSK9 antibody to a patient at a frequency of once every two weeks, if after 8 weeks (i.e., 5 doses administered at Week 0, Week 2 and Week 4, Week 6 and Week 8), the patient has not achieved a serum LDL-C concentration of less than 70 mg/dL, then the dose of anti-PCSK9 antibody is increased to e.g., 150 mg administered once every two weeks thereafter (e.g., starting at Week 10 or Week 12, or later).

In certain embodiments, the antibody or antigen-binding fragment thereof that specifically binds PCSK9 is administered to the patient at a dose of about 75 mg at a frequency of once every two weeks. In certain embodiments, the about 75 mg dose is maintained if the patient's LDL-C measured after one or more, two or more, three or more, four or more, or five or more doses is <70 mg/dL. In certain embodiments, the about 75 mg dose is discontinued if the patient's LDL-C measured after one or more, two or more, three or more, four or more, or five or more doses remains 70 mg/dL, and the antibody or antigen-binding fragment thereof that specifically binds PCSK9 is subsequently administered to the patient at a dose of about 150 mg at a frequency of once every two weeks.

In certain embodiments, the antibody or antigen-binding fragment thereof that specifically binds PCSK9 is administered to the patient at a dose of about 300 mg at a frequency of once every four weeks. In certain embodiments, the about 300 mg dose is maintained if the patient's LDL-C measured after one or more, two or more, three or more, four or more, or five or more doses is <70 mg/dL. In certain embodiments, the about 300 mg dose is discontinued if the patient's LDL-C measured after one or more, two or more, three or more, four or more, or five or more doses remains 70 mg/dL, and the antibody or antigen-binding fragment thereof that specifically binds PCSK9 is subsequently administered to the patient at a dose of about 150 mg at a frequency of once every two weeks.

In certain embodiments, the antibody or antigen-binding fragment thereof that specifically binds PCSK9 is administered to the patient at a dose of about 150 mg at a frequency of once every two weeks.

In certain embodiments, when the antibody or antigen-binding fragment thereof that specifically binds PCSK9 is administered to the patient at a dose of about 150 mg at a frequency of once every two weeks, the about 150 mg dose is discontinued if the patient's LDL-C measured after at least one dose or at least two, three, four, or five consecutive doses is <10, 15, 20, or 25 mg/dL, and the antibody or antigen-binding fragment thereof that specifically binds PCSK9 is subsequently administered to the patient at a dose of about 75 mg at a frequency of once every two weeks. While not wishing to be bound by theory, it is hypothesized that a very low LDL-C level (e.g., <10, 15, 20, or 25 mg/dL) may aggravate diabetes. In certain embodiments, the about 150 mg dose is administered to the patient as a constant dose. In certain embodiments, the about 150 mg dose is administered to the patient after a dose adjustment as disclosed herein (e.g., from about 75 mg every two week, or from about 300 mg every four weeks).

### Combination Therapies

As described elsewhere herein, the methods may comprise administering a PCSK9 inhibitor to a patient in combination with (“on top of”) the patient's previously prescribed lipid modifying therapy (LMT). LMTs include, but are not limited to statins, fibrates, niacin (e.g., nicotinic acid and its derivatives), bile acid sequestrants, ezetimibe, lomitapide, phytosterols, orlistat, etc. For example, a PCSK9 inhibitor may be administered to a patient in combination with a stable daily therapeutic statin regimen. Exemplary daily therapeutic statin regimens that a PCSK9 inhibitor may be administered in combination with in the context of the present methods include, e.g., atorvastatin (10, 20, 40 or 80 mg daily), (atorvastatin/ezetimibe 10/10 or 40/10 mg daily), rosuvastatin (5, 10 or 20 mg daily), cerivastatin (0.4 or 0.8 mg daily), pitavastatin (1, 2 or 4 mg daily), fluvastatin (20, 40 or 80 mg daily), simvastatin (5, 10, 20, 40 or 80 mg daily), simvastatin/ezetimibe (10/10, 20/10, 40/10 or 80/10 mg daily), lovastatin (10, 20, 40 or 80 mg daily), pravastatin (10, 20, 40 or 80 mg daily), and combinations thereof. In certain embodiments, the statin therapy is a maximally tolerated statin therapy for the patient. Other LMTs that a PCSK9 inhibitor may be administered in combination with in the context of the present methods include, e.g., (1) an agent which inhibits cholesterol uptake and or bile acid re-absorption (e.g., ezetimibe); (2) an agent which increase lipoprotein catabolism (such as niacin); and/or (3) activators of the LXR transcription factor that plays a role in cholesterol elimination such as 22-hydroxycholesterol.

According to certain embodiments, methods are provided comprising administering a PCSK9 inhibitor (e.g., an anti-PCSK9 antibody such as alirocumab, evolocumab, bococizumab, lodelcizumab, ralpancizumab or LY3015014) to a patient in combination with an inhibitor of angiopoietin-like protein 3 (e.g., an anti-ANGPTL3 antibody such as REGN1500), an inhibitor of angiopoietin-like protein 4 (e.g., an anti-ANGPTL4 antibody such as the anti-ANGPTL4 antibody referred to in U.S. Pat. No. 9,120,851 as “H1H268P” or “H4H284P”), or an inhibitor of angiopoietin-like protein 8 (e.g., an anti-ANGPTL8 antibody).

According to certain embodiments, methods are provided comprising administering a PCSK9 inhibitor (e.g., an anti-PCSK9 antibody such as alirocumab, evolocumab, bococizumab, lodelcizumab, ralpancizumab or LY3015014) to a patient in combination with an a further anti-diabetic therapy in addition to insulin therapy. Exemplary additional anti-diabetic therapies include without limitation:

(a) all drugs mentioned in the Rote Liste 2016, (e.g. all antidiabetics mentioned in the Rote Liste 2014, chapter 12), all weight-reducing agents or appetite suppressants mentioned in the Rote Liste 2016, chapter 06, all lipid-lowering agents mentioned in the Rote Liste 2016, chapter 58, all antihypertensives mentioned in the Rote Liste 2016 chapter 17, all nephroprotectives mentioned in the Rote Liste, or all diuretics mentioned in the Rote Liste 2016, chapter 36; (b) glucagon like peptide 1 (GLP-1) therapies, including GLP-1, GLP-1 analogues, and GLP-1 receptor agonists, for example: GLP-1(7-37), GLP-1(7-36)amide, lixisenatide (e.g. Lyxumie), exenatide (e.g. exendin-4, rExendin-4, Byetta®, Bydureon®, exenatide NexP), exenatide-LAR, liraglutide (e.g. Victoze), semaglutide, taspoglutide, albiglutide, dulaglutide, albugon, oxyntomodulin, geniproside, ACP-003, CJC-1131, CJC-1134-PC, GSK-2374697, PB-1023, TTP-054, langlenatide (HM-11260C), CM-3, GLP-1 Eligen, AB-201, ORMD-0901, NN9924, NN9926, NN9927, Nodexen, Viador-GLP-1, CVX-096, ZYOG-1, ZYD-1, ZP-3022, CAM-2036, DA-3091, DA-15864, ARI-2651, ARI-2255, exenatide-XTEN (VRS-859), exenatide-XTEN+Glucagon-XTEN (VRS-859+AMX-808) and polymer-bound GLP-1 and GLP-1 analogues; (c) dual GLP-1/GIP agonists (e.g. RG-7697 (MAR-701), MAR-709, BHM081, BHM089, BHM098); dual GLP-1/glucagon receptor agonists (e.g. BHM-034, OAP-189 (PF-05212389, TKS-1225), TT-401/402, ZP2929, LAPS-HMOXM25, MOD-6030);
(d) dual GLP-1/gastrin agonists (e.g. ZP-3022);
(e) gastrointestinal peptides such as peptide YY 3-36 (PYY3-36) or analogues thereof and pancreatic polypeptide (PP) or analogues thereof;
(f) glucagon receptor agonists or antagonists, glucose-dependent insulinotropic polypeptide (GIP) receptor agonists or antagonists, ghrelin antagonists or inverse agonists, xenin and analogues thereof;
(g) dipeptidyl peptidase-IV (DPP-4) inhibitors, for example: alogliptin (e.g. Nesina®, Kazano®, linagliptin (e.g. Ondero®, Trajenta®, Tradjenta®, Trayente), saxagliptin (e.g. Onglyza®′ Komboglyze XR®), sitagliptin (e.g. Januvia®, Xelevia®, Tesavel®, Janumet®, Velmetia®, Juvisync®, Janumet XR®), anagliptin, teneligliptin (e.g. Tenelia®, trelagliptin, vildagliptin (e.g. Galvus®, Galvumet®, gemigliptin, omarigliptin, evogliptin, dutogliptin, DA-1229, MK-3102, KM-223, KRP-104, PBL-1427, Pinoxacin hydrochloride, and Ari-2243;
(h) sodium-dependent glucose transporter 2 (SGLT-2) inhibitors, for example: canagliflozin, dapagliflozin, remogliflozin, remogliflozin etabonate, sergliflozin, empagliflozin, ipragliflozin, tofogliflozin, luseogliflozin, ertugliflozin, EGT-0001442, LIK-066, SBM-TFC-039, and KGA-3235 (DSP-3235);
(i) dual inhibitors of SGLT-2 and SGLT-1 (e.g. LX-4211, LIK066);
(j) SGLT-1 inhibitors (e.g. LX-2761, KGA-3235) or SGLT-1 inhibitors in combination with anti-obesity drugs such as ileal bile acid transfer (IBAT) inhibitors (e.g. GSK-1614235+GSK-2330672);
(k) biguanides (e.g. metformin, buformin, phenformin);
(l) thiazolidinediones (e.g. pioglitazone, rosiglitazone), glitazone analogues (e.g. lobeglitazone);
(m) peroxisome proliferator-activated receptors (PPAR−)(alpha, gamma or alpha/gamma) agonists or modulators (e.g. saroglitazar (e.g. Lipaglye), GFT-505), or PPAR gamma partial agonists (e.g. Int-131);
(n) sulfonylureas (e.g. tolbutamide, glibenclamide, glimepiride, Amaryl®, glipizide) and meglitinides (e.g. nateglinide, repaglinide, mitiglinide);
(o) alpha-glucosidase inhibitors (e.g. acarbose, miglitol, voglibose);
(q) amylin and amylin analogues (e.g. pramlintide, Symlie);
(p) G-protein coupled receptor 119 (GPR119) agonists (e.g. GSK-1292263, PSN-821, MBX-2982, APD-597, ARRY-981, ZYG-19, DS-8500, HM-47000, YH-Chem1);
(q) GPR40 agonists (e.g. TUG-424, P-1736, P-11187, JTT-851, GW9508, CNX-011-67, AM-1638, AM-5262);
(r) GPR120 agonists and GPR142 agonists;
(s) systemic or low-absorbable TGR5 (GPBAR1=G-protein-coupled bile acid receptor 1) agonists (e.g. INT-777, XL-475, SB756050);
(t) diabetes immunotherapeutics, for example: oral C—C chemokine receptor type 2 (CCR-2) antagonists (e.g. CCX-140, JNJ-41443532), interleukin 1 beta (IL-1β) antagonists (e.g. AC-201), or oral monoclonal antibodies (MoA) (e.g. methalozamide, VVP808, PAZ-320, P-1736, P F-05175157, P F-04937319);
(v) anti-inflammatory agents for the treatment of the metabolic syndrome and diabetes, for example: nuclear factor kappa B inhibitors (e.g. Triolee);
(w) adenosine monophosphate-activated protein kinase (AMPK) stimulants, for example: Imeglimin (PXL-008), Debio-0930 (MT-63-78), R-118;
(x) inhibitors of 11-beta-hydroxysteroid dehydrogenase 1 (11-beta-HSD-1) (e.g. LY2523199, BMS770767, RG-4929, BMS816336, AZD-8329, HSD-016, BI-135585);
(y) activators of glucokinase (e.g. PF-04991532, TTP-399 (GK1-399), GKM-001 (ADV-1002401), ARRY-403 (AMG-151), TAK-329, TMG-123, ZYGK1);
(z) inhibitors of diacylglycerol O-acyltransferase (DGAT) (e.g. pradigastat (LCQ-908)), inhibitors of protein tyrosine phosphatase 1 (e.g. trodusquemine), inhibitors of glucose-6-phosphatase, inhibitors of fructose-1,6-bisphosphatase, inhibitors of glycogen phosphorylase, inhibitors of phosphoenol pyruvate carboxykinase, inhibitors of glycogen synthase kinase, inhibitors of pyruvate dehydrogenase kinase;
(aa) modulators of glucose transporter-4, somatostatin receptor 3 agonists (e.g. MK-4256);
(bb) one or more lipid lowering agents are also suitable as combination partners, for example: 3-hydroxy-3-methylglutaryl-coenzym-A-reductase (HMG-CoA-reductase) inhibitors such as simvastatin (e.g. Zocor®, Inegy®, Simcor®), atorvastatin (e.g. Sortis®, Caduee), rosuvastatin (e.g. Crestoe), pravastatin (e.g. Lipostat®, Seliprae), fluvastatin (e.g. Lescol®), pitavastatin (e.g. Livazo®, Livale), lovastatin (e.g. Mevacor®, Advicor®), mevastatin (e.g. Compactie), rivastatin, cerivastatin (Lipobay®), fibrates such as bezafibrate (e.g. Cedur® retard), ciprofibrate (e.g. Hyperlipen®), fenofibrate (e.g. Antara®, Lipofen®, Lipanthyr), gemfibrozil (e.g. Lopid®,) Gevilon®, etofibrate, simfibrate, ronifibrate, clinofibrate, clofibride, nicotinic acid and derivatives thereof (e.g. niacin, including slow release formulations of niacin), nicotinic acid receptor 1 agonists (e.g. GSK-256073), PPAR-delta agonists, acetyl-CoA-acetyltransferase (ACAT) inhibitors (e.g. avasimibe), cholesterol absorption inhibitors (e.g. ezetimibe, Ezetrol®, Zetia®, Liptruzet®, Vytorin®, S-556971), bile acid-binding substances (e.g. cholestyramine, colesevelam), ileal bile acid transport (IBAT) inhibitors (e.g. GSK-2330672, LUM-002), microsomal triglyceride transfer protein (MTP) inhibitors (e.g. lomitapide (AEGR-733), SLx-4090, granotapide), modulators of proprotein convertase subtilisin/kexin type 9 (PCSK9) (e.g. alirocumab (REGN727/SAR236553), AMG-145, LGT-209, PF-04950615, MPSK31 69A, LY3015014, ALD-306, ALN-PCS, BMS-962476, SPC5001, ISIS-394814, IB20, LGT-210, ID05, BMS-PCSK9Rx-2, SX-PCK9, RG7652), LDL receptor up-regulators, for example liver selective thyroid hormone receptor beta agonists (e.g. eprotirome (KB-2115), MB07811, sobetirome (QRX-431), VIA-3196, ZYT1), HDL-raising compounds such as: cholesteryl ester transfer protein (CETP) inhibitors (e.g. anacetrapib (MK0859), dalcetrapib, evacetrapib, JTT-302, DRL-17822, TA-8995, R-1658, LY-2484595, DS-1442), or dual CETP/PCSK9 inhibitors (e.g. K-312), ATP-binding cassette (ABC1) regulators, lipid metabolism modulators (e.g. BMS-823778, TAP-301, DRL-21994, DRL-21995), phospholipase A2 (PLA2) inhibitors (e.g. darapladib, Tyrisa®, varespladib, rilapladib), ApoA-I enhancers (e.g. RVX-208, CER-001, MDCO-216, CSL-112), cholesterol synthesis inhibitors (e.g. ETC-1002), lipid metabolism modulators (e.g. BMS-823778, TAP-301, DRL-21994, DRL-21995) and omega-3 fatty acids and derivatives thereof (e.g. icosapent ethyl (AMR101), Epanova®, AKR-063, NKPL-66, PRC-4016, CAT-2003); (cc) bromocriptine (e.g. Cycloset®, Parlodel®, phentermine and phentermine formulations or combinations (e.g. Adipex-P, Ionamin, Qsymia®), benzphetamine (e.g. Didrex®), diethylpropion (e.g. Tenuate®), phendimetrazin (e.g. Adipost®, Bontril®,) bupropion and combinations (e.g. Zyban®, Wellbutrin XL®, Contrave®, Empatic®), sibutramine (e.g. Reductil®, Meridia®), topiramat (e.g. Topamax®), zonisamid (e.g. Zonegran®), tesofensine, opioid antagonists such as naltrexone (e.g. Naltrexin®, naltrexone+bupropion), cannabinoid receptor 1 (CB1) antagonists (e.g. TM-38837), melanin-concentrating hormone (MCH-1) antagonists (e.g. BMS-830216, ALB-127158(a)), MC4 receptor agonists and partial agonists (e.g. AZD-2820, RM-493), neuropeptide Y5 (NPY5) or NPY2 antagonists (e.g. velneperit, S-234462), NPY4 agonists (e.g. PP-1420), beta-3-adrenergic receptor agonists, leptin or leptin mimetics, agonists of the 5-hydroxytryptamine 2c (5HT2c) receptor (e.g. lorcaserin, Belviq®), pramlintide/metreleptin, lipase inhibitors such as cetilistat (e.g. Cametoe), orlistat (e.g. Xenical®, Calobalie), angiogenesis inhibitors (e.g. ALS-L1023), betahistidin and histamine H3 antagonists (e.g. HPP-404), AgRP (agouti related protein) inhibitors (e.g. TTP-435), serotonin re-uptake inhibitors such as fluoxetine (e.g. Fluctine), duloxetine (e.g. Cymbalte), dual or triple monoamine uptake inhibitors (dopamine, norepinephrine and serotonin re-uptake) such as sertraline (e.g. Zolofe), tesofensine, methionine aminopeptidase 2 (MetAP2) inhibitors (e.g. beloranib), and antisense oligonucleotides against production of fibroblast growth factor receptor 4 (FGFR4) (e.g. ISIS-FGFR4Rx) or prohibitin targeting peptide-1 (e.g. Adipotide); and
(dd) nitric oxide donors, AT1 antagonists or angiotensin II (AT2) receptor antagonists such as telmisartan (e.g. Kinzal®, Micardie), candesartan (e.g. Atacand®, Bloprese), valsartan (e.g. Diovan®, Co-Diovan®), losartan (e.g. Cosaar®), eprosartan (e.g. Tevetee), irbesartan (e.g. Aprovel®, CoAprover), olmesartan (e.g. Votum®, Olmetee), tasosartan, azilsartan (e.g. Edarbr), dual angiotensin receptor blockers (dual ARBs), angiotensin converting enzyme (ACE) inhibitors, ACE-2 activators, renin inhibitors, prorenin inhibitors, endothelin converting enzyme (ECE) inhibitors, endothelin receptor (ET1/ETA) blockers, endothelin antagonists, diuretics, aldosterone antagonists, aldosterone synthase inhibitors, alpha-blockers, antagonists of the alpha-2 adrenergic receptor, beta-blockers, mixed alpha-/beta-blockers, calcium antagonists, calcium channel blockers (CCBs), nasal formulations of the calcium channel blocker diltiazem (e.g. CP-404), dual mineralocorticoid/CCBs, centrally acting antihypertensives, inhibitors of neutral endopeptidase, aminopeptidase-A inhibitors, vasopeptide inhibitors, dual vasopeptide inhibitors such as neprilysin-ACE inhibitors or neprilysin-ECE inhibitors, dual-acting AT receptor-neprilysin inhibitors, dual AT1/ETA antagonists, advanced glycation end-product (AGE) breakers, recombinant renalase, blood pressure vaccines such as anti-RAAS (renin-angiotensin-aldosteron-system) vaccines, AT1- or AT2-vaccines, drugs based on hypertension pharmacogenomics such as modulators of genetic polymorphisms with antihypertensive response, thrombocyte aggregation inhibitors, and others; and (ee) combinations thereof that are suitable.

In certain embodiments, the additional anti-diabetic therapy is a GLP-1 therapy (e.g., lixisenatide). In certain embodiments, the GLP-1 therapy is formulated with methionine (e.g., L-methionine or D-methionine). In certain embodiments, polysorbate (e.g., polysorbate 20, polysorbate 80), poloxamer (e.g., poloxamer 188), benzalkonium chloride, histidine, lysine, and/or EDTA are absent or substantially absent from the formulation of the GLP-1 therapy. In certain embodiments, the formulation of the GLP-1 therapy is free or substantially free of surfactants, such as polyols (e.g., polypropylene glycols, polyethylene glycols, poloxamers, Pluronics, Tetronics), partial and fatty acid esters and ethers of polyhydric alcohols such as those of glycerol and sorbitol (e.g., Span®, Tween®, Myrj®, Brij®, Cremophor®). The formulation of the GLP-1 therapy can comprise a suitable preservative (e.g., phenol, m-cresol, benzyl alcohol, and/or p-hydroxybenzoate esters) and suitable tonicity modifiers (e.g., glycerol, dextrose, lactose, sorbitol, mannitol, glucose, NaCl, calcium or magnesium compounds such as CaCl₂). The concentrations of glycerol, dextrose, lactose, sorbitol, mannitol, and glucose are customarily in the range of 100-250 mM, NaCl in a concentration of up to 150 mM.

In certain embodiments, the insulin therapy that the patient receives is combined with the additional anti-diabetic therapy (e.g., any of the foregoing anti-diabetic therapies which are not insulin therapies). For example, in certain embodiments, the anti-diabetic therapy comprises a combination of an insulin therapy (e.g., insulin glargine) and a GLP-1 therapy (e.g., lixisenatide). These therapies can be provided either separately or in a single pharmaceutical composition. For example, insulin glargine and lixisenatide can be formulated in a single pharmaceutical composition (e.g., Soliqua® 100/33) for daily injection.

In the context of the methods, additional therapeutically active component(s), e.g., any of the agents listed above or derivatives thereof, may be administered just prior to, concurrent with, or shortly after the administration of a PCSK9 inhibitor; (for purposes of the present disclosure, such administration regimens are considered the administration of a PCSK9 inhibitor “in combination with” an additional therapeutically active component). The present methods include pharmaceutical compositions and methods of use thereof in which a PCSK9 inhibitor is co-formulated with one or more of the additional therapeutically active component(s) as described elsewhere herein.

### Administration of a PCSK9 Inhibitor as Add-on Therapy

The present methods of treatment including treating a patient with hypercholesterolemia and diabetes with a PCSK9 inhibitor, such as an antibody or antigen-binding fragment thereof that specifically binds PCKS9, wherein the PCSK9 inhibitor can be administered as an add-on to the patient's pre-existing insulin therapy and/or LMT (if applicable), such as an add-on to the patient's pre-existing daily therapeutic insulin and/or statin regimen.

For example, the methods include add-on therapeutic regimens wherein the PCSK9 inhibitor is administered as add-on therapy to the same stable multiple daily insulin regimen and/or daily therapeutic statin regimen (i.e., same dosing amount of statin) that the patient was on prior to receiving the PCSK9 inhibitor. In other embodiments, the PCSK9 inhibitor is administered as add-on therapy to a therapeutic insulin and/or statin regimen comprising an insulin and/or a statin in an amount that is more than or less than the dose of insulin and/or stain the patient was on prior to receiving the PCSK9 inhibitor. For example, after starting a therapeutic regimen comprising a PCSK9 inhibitor administered at a particular dosing frequency and amount, the daily dose of insulin and/or statin administered or prescribed to the patient may (a) stay the same, (b) increase, or (c) decrease (e.g., up-titrate or down-titrate) in comparison to the daily statin dose the patient was taking before starting the PCSK9 inhibitor therapeutic regimen, depending on the therapeutic needs of the patient.

### Therapeutic Efficacy

The methods result in the reduction in serum levels of one or more lipid component selected from the group consisting of LDL-C, ApoB, ApoB100, non-HDL-C, total cholesterol, VLDL-C, triglycerides, Lp(a), HDL-C, LDL particle number, LDL particle size, ApoC3, ApoA-1, triglyceride-rich lipoprotein cholesterol (TRL-C), and remnant cholesterol. According to certain embodiments, administration of a pharmaceutical composition comprising a PCSK9 inhibitor to a patient will result in a mean percent reduction from baseline in serum low density lipoprotein cholesterol (LDL-C) of at least about 25%, 30%, 35%, 40%, 45%, 50%, 55%, 60%, or greater; a mean percent reduction from baseline in ApoB of at least about 25%, 30%, 35%, 40%, 45%, 50%, 55%, 60%, or greater; a mean percent reduction from baseline in ApoB100 of at least about 25%, 30%, 35%, 40%, 45%, 50%, 55%, 60%, or greater; a mean percent reduction from baseline in non-HDL-C of at least about 25%, 30%, 35%, 40%, 45%, 50%, 55%, 60%, or greater; a mean percent reduction from baseline in total cholesterol of at least about 10%, 15%, 20%, 25%, 30%, 35%, 40%, or greater; a mean percent reduction from baseline in VLDL-C of at least about 5%, 10%, 15%, 20%, 25%, 30%, or greater; a mean percent reduction from baseline in triglycerides of at least about 5%, 10%, 15%, 20%, 25%, 30%, 35% or greater; a mean percentage reduction from baseline in the number of LDL particles of at least about 20%, 25%, 30%, 35%, 40%, 45%, 50% or greater; a mean percent reduction from baseline in the size of LDL particles of at least about 1.5%, 2%, 2.5%, 3%, 3.5% or 4% or more; a mean percentage reduction from baseline in apolipoprotein C3 (ApoC3) of at least about 5%, 5.5%, 6.0%, 6.5%, 7.0%, 7.5%, 8.0%, 9.0%, 10% or more; a mean percent increase from baseline in HDL-C of at least about 1%, 2%, 3%, 4%, 5% or greater; a mean percent increase from baseline in ApoA-1 of at least about 1%, 2%, 3%, 4%, 5% or greater; a mean percentage reduction from baseline in TRL-C of at least about 5%, 10%, 15%, 20%, 25%, 30%, or greater; and/or a mean percent reduction from baseline in Lp(a) of at least about 5%, 10%, 15%, 20%, 25%, or greater.

The present methods include treating a patient with hypercholesterolemia and T1 DM that is receiving insulin therapy, the methods comprising administering multiple doses of an anti-PCSK9 antibody or antigen binding fragment thereof to the patient at a dosing amount of about 75 to 150 mg per dose, and a dosing frequency of about once every two weeks or every four weeks, or a dosing regimen in accordance with an up-titration dosing regimen as disclosed herein. After about 8, 10, 12, 14, 16, 18, 20, 22, 24 or more weeks of treatment with the anti-PCSK9 antibody, the patient may exhibit a reduction in LDL-C level from baseline of at least 35%, 50%, or 60%. In certain embodiments, following one or more weeks of treatment with the anti-PCSK9 antibody, the patient exhibits a reduction in LDL-C level from baseline of about 35%, 50%, or 60%, or more.

The present methods also include treating a patient with hypercholesterolemia and T2DM that is receiving insulin therapy, the methods comprising administering multiple doses of an anti-PCSK9 antibody or antigen binding fragment thereof to the patient at a dosing amount of about 75 to 150 mg per dose, and a dosing frequency of about once every two weeks or every four weeks, or a dosing regimen in accordance with an up-titration dosing regimen as disclosed herein. After about 8, 10, 12, 14, 16, 18, 20, 22, 24 or more weeks of treatment with the anti-PCSK9 antibody, the patient may exhibit a reduction in LDL-C level from baseline of at least 40%, 48%, or 54%. In certain embodiments, following one or more weeks of treatment with the anti-PCSK9 antibody, the patient exhibits a reduction in LDL-C level from baseline of about 40%, 48%, or 54%, or more.

As disclosed herein, the present methods do not alter the patient's diabetic parameters. For example, in certain embodiments, the method does not affect (e.g., does not change by greater than 1%, 2%, 3%, 4%, 5%, 6%, 7%, 8%, 9%, or 10%) the hemoglobin A1c (HbA1c) level of the patient. In certain embodiments, the method does not affect (e.g., does not change by greater than 2%, 4%, 6%, 8%, 10%, 12%, 15%, 18%, or 20%) the fasting plasma glucose (FPG) level of the patient.

In further embodiments the present invention relates to uses of an antibody or an antigen-binding fragment thereof which specifically binds human proprotein convertase subtilisin/kexin type 9 (PCSK9), for treating hypercholesterolemia in a patient with type 1 diabetes mellitus (T1 DM).

In yet further embodiments the present invention relates to methods for treating hypercholesterolemia in a patient with type 1 diabetes mellitus (T1 DM).

In one embodiment said use and/or method comprises the steps:

(a) selecting a high cardiovascular risk patient receiving insulin therapy that has

(i) T1 DM, and

(ii) hypercholesterolemia not adequately controlled by maximally tolerated statin therapy; and

(b) administering to the patient 75 mg, 150 mg or 300 mg of an antibody or an antigen-binding fragment thereof which specifically binds human proprotein convertase subtilisin/kexin type 9 (PCSK9), wherein the patient receives a concomitant insulin therapy.

In one embodiment of said use and/or method the 75 mg of the antibody or antigen binding fragment is administered to the patient every two weeks.

In one embodiment of said use and/or method the 150 mg of the antibody or antigen binding fragment is administered to the patient every two weeks.

In one embodiment of said use and/or method the 300 mg of the antibody or antigen binding fragment is administered to the patient every four weeks.

In one embodiment of said use and/or method the antibody or antigen-binding fragment thereof comprises the three heavy chain CDRs set forth in SEQ ID NOs: 2, 3, and 4, and the three light chain CDRs set forth in SEQ ID NOs: 7, 8, and 10.

In one embodiment of said use and/or method the antibody or antigen-binding fragment thereof comprises a heavy chain variable region (HCVR) having the amino acid sequence of SEQ ID NO: 1 and a light chain variable region (LCVR) having the amino acid sequence of SEQ ID NO: 6.

In one embodiment of said use and/or method the antibody or antigen-binding fragment thereof is selected from the group consisting of alirocumab, evolocumab, bococizumab, lodelcizumab, ralpancizumab and LY3015014.

In one embodiment of said use and/or method the antibody or antigen-binding fragment thereof is alirocumab.

In one embodiment said use and/or method comprises further the steps:

(c) administering to the patient one or more following doses of 75 mg of the antibody or antigen-binding fragment thereof about every two weeks if the LDL-C level in the patient is lower than a threshold level, or administering one or more following doses of 150 mg of the antibody or antigen-binding fragment thereof about every two weeks if the LDL-C level in the patient is greater than or equal to the threshold level.

In one embodiment said use and/or method comprises further the step:

(c) administering to the patient one or more following doses of 300 mg of the antibody or antigen-binding fragment thereof about every four weeks if the LDL-C level in the patient is lower than a threshold level, or administering one or more following doses of 150 mg of the antibody or antigen-binding fragment thereof about every two weeks if the LDL-C level in the patient is greater than or equal to the threshold level.

In one embodiment of said use and/or method the threshold level is 70 mg/dL.

In one embodiment of said use and/or method the antibody or antigen-binding fragment thereof is administered subcutaneously.

In one embodiment of said use and/or method the patient further receives a concomitant lipid-modifying therapy (LMT).

In one embodiment of said use and/or method the LMT is selected from the group consisting of a statin, a cholesterol absorption inhibitor, a fibrate, niacin, an omega-3 fatty acid, and a bile acid sequestrant.

In one embodiment of said use and/or method the LMT is a statin therapy.

In one embodiment of said use and/or method the statin is selected from the group consisting of atorvastatin, rosuvastatin, simvastatin, pravastatin, lovastatin, fluvastatin, pitavastatin, and cerivastatin.

In one embodiment of said use and/or method the statin therapy is a maximally tolerated statin therapy.

In one embodiment of said use and/or method the cholesterol absorption inhibitor is ezetimibe.

In one embodiment of said use and/or method the patient is intolerant to a statin.

In one embodiment of said use and/or method the insulin therapy is selected from the group consisting of human insulin, Insulin glargine, insulin glulisine, insulin detemir, insulin lispro, insulin degludec, insulin aspart, and basal insulin.

In one embodiment of said use and/or method the patient receives a concomitant anti-diabetic therapy in addition to insulin therapy.

In one embodiment of said use and/or method the additional concomitant anti-diabetic therapy is selected from the group consisting of a glucagon like peptide 1 (GLP-1) therapy, a gastrointestinal peptide, a glucagon receptor agonist or antagonist, a glucose-dependent insulinotropic polypeptide (GIP) receptor agonist or antagonist, a ghrelin antagonist or inverse agonist, xenin, a xenin analogue, a biguanide, a sulfonylurea, a meglitinide, a thiazolidinedione, a DPP-4 inhibitor, an alpha-glucosidase inhibitor, a sodium-dependent glucose transporter 2 (SGLT-2) inhibitor, an SGLT-1 inhibitor, a peroxisome proliferator-activated receptor (PPAR−)(alpha, gamma or alpha/gamma) agonist or modulator, amylin, an amylin analogue, a G-protein coupled receptor 119 (GPR119) agonist, a GPR40 agonist, a GPR120 agonist, a GPR142 agonist, a systemic or low-absorbable TGR5 agonist, a diabetes immunotherapeutic, an anti-inflammatory agents for the treatment of the metabolic syndrome and diabetes, an adenosine monophosphate-activated protein kinase (AMPK) stimulant, an inhibitor of 11-beta-hydroxysteroid dehydrogenase 1, an activator of glucokinase, an inhibitor of diacylglycerol O-acyltransferase (DGAT), a modulator of glucose transporter-4, a somatostatin receptor 3 agonist, a lipid lowering agent, and a combination thereof.

In one embodiment of said use and/or method the antibody or antigen-binding fragment thereof reduces the LDL-C level of the patient by at least 30%, 35%, 40%, or 45%.

In one embodiment of said use and/or method the antibody or antigen-binding fragment thereof reduces the non-HDL-C level of the patient by at least 25%, 30%, 35%, or 40%.

In one embodiment of said use and/or method the antibody or antigen-binding fragment thereof reduces the apolipoprotein C3 (ApoC3) level of the patient.

In one embodiment of said use and/or method the antibody or antigen-binding fragment thereof reduces the number and/or size of lipoprotein particles in the patient.

In one embodiment of said use and/or method the antibody or antigen-binding fragment thereof:

(a) does not affect the hemoglobin A1c (HbA1c) level of the patient; and/or
(b) does not affect the fasting plasma glucose (FPG) level of the patient.

In further embodiments the present inventions relate to uses and/or methods for treating hypercholesterolemia in a patient with type 1 diabetes mellitus (T1 DM), the method comprising the steps:
(a) selecting a high cardiovascular risk patient receiving insulin therapy that has
   (i) T1 DM, and
   (ii) hypercholesterolemia not adequately controlled by maximally tolerated statin therapy;
(b) administering every two weeks to the patient 75 mg of an antibody or an antigen-binding fragment thereof which specifically binds human proprotein convertase subtilisin/kexin type 9 (PCSK9); and
(c) administering to the patient one or more following doses of 75 mg of the antibody or antigen-binding fragment thereof about every two weeks if the LDL-C level in the patient is lower than 70 mg/dL, or administering one or more following doses of 150 mg of the antibody or antigen-binding fragment thereof about every two weeks if the LDL-C level in the patient is greater than or equal to 70 mg/dL,
wherein the antibody or antigen-binding fragment thereof comprises an HCVR having the amino acid sequence of SEQ ID NO: 1 and an LCVR having the amino acid sequence of SEQ ID NO: 6, and wherein the patient receives a concomitant insulin therapy.

In further embodiments the present inventions relate to uses and/or methods for treating hypercholesterolemia in a patient with type 2 diabetes mellitus (T2DM), the method comprising the steps:
(a) selecting a high cardiovascular risk patient receiving insulin therapy that has
   (i) T2DM, and
   (ii) hypercholesterolemia not adequately controlled by maximally tolerated statin therapy; and
(b) administering to the patient 75 mg, 150 mg or 300 mg of an antibody or an antigen-binding fragment thereof which specifically binds human proprotein convertase subtilisin/kexin type 9 (PCSK9), wherein the patient receives a concomitant insulin therapy.

In one embodiment of said use and/or method the 75 mg of the antibody or antigen binding fragment is administered to the patient every two weeks.

In one embodiment of said use and/or method the 150 mg of the antibody or antigen binding fragment is administered to the patient every two weeks.

In one embodiment of said use and/or method the 300 mg of the antibody or antigen binding fragment is administered to the patient every four weeks.

In one embodiment of said use and/or method the antibody or antigen-binding fragment thereof comprises the three heavy chain CDRs set forth in SEQ ID NOs: 2, 3, and 4, and the three light chain CDRs set forth in SEQ ID NOs: 7, 8, and 10.

In one embodiment of said use and/or method the antibody or antigen-binding fragment thereof comprises a heavy chain variable region (HCVR) having the amino acid sequence of SEQ ID NO: 1 and a light chain variable region (LCVR) having the amino acid sequence of SEQ ID NO: 6.

In one embodiment of said use and/or method the antibody or antigen-binding fragment thereof is selected from the group consisting of alirocumab, evolocumab, bococizumab, lodelcizumab, ralpancizumab, and LY3015014.

In one embodiment of said use and/or method the antibody or antigen-binding fragment thereof is alirocumab.

In one embodiment of said use and/or method further comprises the step:
(c) administering to the patient one or more following doses of 75 mg of the antibody or antigen-binding fragment thereof about every two weeks if the LDL-C level in the patient is lower than the threshold level, or administering one or more following doses of 150 mg of the antibody or antigen-binding fragment thereof about every two weeks if the LDL-C level in the patient is greater than or equal to the threshold level.

In one embodiment of said use and/or method further comprises the step:
(c) administering to the patient one or more following doses of 300 mg of the antibody or antigen-binding fragment thereof about every four weeks if the LDL-C level in the patient is lower than a threshold level, or administering one or more following doses of 150 mg of the antibody or antigen-binding fragment thereof about every two weeks if the LDL-C level in the patient is greater than or equal to the threshold level.

In one embodiment of said use and/or method the threshold level is 70 mg/dL.

In one embodiment of said use and/or method the antibody or antigen-binding fragment thereof is administered subcutaneously.

In one embodiment of said use and/or method the patient further receives a concomitant lipid-modifying therapy (LMT).

In one embodiment of said use and/or method the LMT is selected from the group consisting of a statin, a cholesterol absorption inhibitor, a fibrate, niacin, an omega-3 fatty acid, and a bile acid sequestrant.

In one embodiment of said use and/or method the LMT is a statin therapy.

In one embodiment of said use and/or method the statin is selected from the group consisting of atorvastatin, rosuvastatin, simvastatin, pravastatin, lovastatin, fluvastatin, pitavastatin, and cerivastatin.

In one embodiment of said use and/or method the statin therapy is a maximally tolerated dose statin therapy.

In one embodiment of said use and/or method the cholesterol absorption inhibitor is ezetimibe.

In one embodiment of said use and/or method the patient is intolerant to a statin.

In one embodiment of said use and/or method the insulin therapy is selected from the group consisting of human insulin, Insulin glargine, insulin glulisine, insulin detemir, insulin lispro, insulin degludec, insulin aspart, and basal insulin.

In one embodiment of said use and/or method the patient receives aconcomitant anti-diabetic therapy in addition to insulin therapy.

In one embodiment of said use and/or method the additional anti-diabetic therapy is selected from the group consisting of an a glucagon like peptide 1 (GLP-1) therapy, a gastrointestinal peptide, a glucagon receptor agonist or antagonist, a glucose-dependent insulinotropic polypeptide (GIP) receptor agonist or antagonist, a ghrelin antagonist or inverse agonist, xenin, a xenin analogue, a biguanide, a sulfonylurea, a meglitinide, a thiazolidinedione, a DPP-4 inhibitor, an alpha-glucosidase inhibitor, a sodium-dependent glucose transporter 2 (SGLT-2) inhibitor, an SGLT-1 inhibitor, a peroxisome proliferator-activated receptor (PPAR−)(alpha, gamma or alpha/gamma) agonist or modulator, amylin, an amylin analogue, a G-protein coupled receptor 119 (GPR119) agonist, a GPR40 agonist, a GPR120 agonist, a GPR142 agonist, a systemic or low-absorbable TGR5 agonist, a diabetes immunotherapeutic, an anti-inflammatory agents for the treatment of the metabolic syndrome and diabetes, an adenosine monophosphate-activated protein kinase (AMPK) stimulant, an inhibitor of 11-beta-hydroxysteroid dehydrogenase 1, an activator of glucokinase, an inhibitor of diacylglycerol O-acyltransferase (DGAT), a modulator of glucose transporter-4, a somatostatin receptor 3 agonist, a lipid lowering agent, and a combination thereof.

In one embodiment of said use and/or method the antibody or antigen-binding fragment thereof reduces the LDL-C level of the patient by at least 30%, 35%, 40%, or 45%.

In one embodiment of said use and/or method the antibody or antigen-binding fragment thereof reduces the non-HDL-C level of the patient by at least 20%, 25%, 30%, or 35%.

In one embodiment of said use and/or method the antibody or antigen-binding fragment thereof reduces the ApoC3 level of the patient.

In one embodiment of said use and/or method the antibody or antigen-binding fragment thereof reduces the number and/or size of lipoprotein particles in the patient.

In one embodiment of said use and/or method the antibody or antigen-binding fragment thereof:

(a) does not affect the hemoglobin A1c (HbA1c) level of the patient; and/or
(b) does not affect the fasting plasma glucose (FPG) level of the patient.

In further embodiments the present inventions relate to uses and/or methods for treating hypercholesterolemia in a patient with type 2 diabetes mellitus (T2DM), the method comprising the steps:
(a) selecting a high cardiovascular risk patient receiving insulin therapy that has
   (i) T2DM, and
   (ii) hypercholesterolemia not adequately controlled by maximally tolerated statin therapy;
(b) administering every two weeks to the patient 75 mg of an antibody or an antigen-binding fragment thereof which specifically binds human proprotein convertase subtilisin/kexin type 9 (PCSK9); and
(c) administering to the patient one or more following doses of 75 mg of the antibody or antigen-binding fragment thereof about every two weeks if the LDL-C level in the patient is lower than 70 mg/dL, or administering one or more following doses of 150 mg of the antibody or antigen-binding fragment thereof about every two weeks if the LDL-C level in the patient is greater than or equal to 70 mg/dL, wherein the antibody or antigen-binding fragment thereof comprises an HCVR having the amino acid sequence of SEQ ID NO: 1 and an LCVR having the amino acid sequence of SEQ ID NO: 6, and wherein the patient receives a concomitant insulin therapy.

In further embodiments the present invention relates to uses of an antibody or an antigen-binding fragment thereof which specifically binds human proprotein convertase subtilisin/kexin type 9 (PCSK9), for treating hypercholesterolemia in a patient with type 2 diabetes mellitus (T2DM) and atherosclerotic cardiovascular disease (ASCVD).

In yet further embodiments the present invention relates to methods for treating hypercholesterolemia in a patient with T2DM and ASCVD.

In one embodiment said use and/or method comprises the steps:

(a) selecting a high cardiovascular risk patient receiving insulin therapy that has

### (i) T2DM,

### (ii) ASCVD, and

(iii) hypercholesterolemia not adequately controlled by maximally tolerated statin therapy; and
(b) administering to the patient 75 mg, 150 mg or 300 mg of an antibody or an antigen-binding fragment thereof which specifically binds human proprotein convertase subtilisin/kexin type 9 (PCSK9), wherein the patient receives a concomitant insulin therapy.

In one embodiment of said use and/or method the ASCVD is defined as coronary heart disease (CHD), ischemic stroke, or peripheral arterial disease.

In one embodiment of said use and/or method the CHD comprises acute myocardial infarction, silent myocardial infarction, and unstable angina.

In one embodiment of said use and/or method the 75 mg of the antibody or antigen binding fragment is administered to the patient every two weeks.

In one embodiment of said use and/or method the 150 mg of the antibody or antigen binding fragment is administered to the patient every two weeks.

In one embodiment of said use and/or method the 300 mg of the antibody or antigen binding fragment is administered to the patient every four weeks.

In one embodiment of said use and/or method the antibody or antigen-binding fragment thereof comprises the three heavy chain CDRs set forth in SEQ ID NOs: 2, 3, and 4, and the three light chain CDRs set forth in SEQ ID NOs: 7, 8, and 10.

In one embodiment of said use and/or method the antibody or antigen-binding fragment thereof comprises a heavy chain variable region (HCVR) having the amino acid sequence of SEQ ID NO: 1 and a light chain variable region (LCVR) having the amino acid sequence of SEQ ID NO: 6.

In one embodiment of said use and/or method the antibody or antigen-binding fragment thereof is selected from the group consisting of alirocumab, evolocumab, bococizumab, lodelcizumab, ralpancizumab, and LY3015014.

In one embodiment of said use and/or method the antibody or antigen-binding fragment thereof is alirocumab.

In one embodiment said use and/or method further comprises the step:

(c) administering to the patient one or more following doses of 75 mg of the antibody or antigen-binding fragment thereof about every two weeks if the LDL-C level in the patient is lower than the threshold level, or administering one or more following doses of 150 mg of the antibody or antigen-binding fragment thereof about every two weeks if the LDL-C level in the patient is greater than or equal to the threshold level.

In one embodiment said use and/or method further comprises the step:

(c) administering to the patient one or more following doses of 300 mg of the antibody or antigen-binding fragment thereof about every four weeks if the LDL-C level in the patient is lower than a threshold level, or administering one or more following doses of 150 mg of the antibody or antigen-binding fragment thereof about every two weeks if the LDL-C level in the patient is greater than or equal to the threshold level.

In one embodiment of said use and/or method the threshold level is 70 mg/dL.

In one embodiment of said use and/or method the antibody or antigen-binding fragment thereof is administered subcutaneously.

In one embodiment of said use and/or method the patient further receives a concomitant lipid-modifying therapy (LMT).

In one embodiment of said use and/or method the LMT is selected from the group consisting of a statin, a cholesterol absorption inhibitor, a fibrate, niacin, an omega-3 fatty acid, and a bile acid sequestrant.

In one embodiment of said use and/or method the LMT is a statin therapy.

In one embodiment of said use and/or method the statin is selected from the group consisting of atorvastatin, rosuvastatin, simvastatin, pravastatin, lovastatin, fluvastatin, pitavastatin, and cerivastatin.

In one embodiment of said use and/or method the statin therapy is a maximally tolerated dose statin therapy.

In one embodiment of said use and/or method the cholesterol absorption inhibitor is ezetimibe.

In one embodiment of said use and/or method the patient is intolerant to a statin.

In one embodiment of said use and/or method the insulin therapy is selected from the group consisting of human insulin, Insulin glargine, insulin glulisine, insulin detemir, insulin lispro, insulin degludec, insulin aspart, and basal insulin.

In one embodiment of said use and/or method the patient receives a concomitant anti-diabetic therapy in addition to insulin therapy.

In one embodiment of said use and/or method the additional anti-diabetic therapy is selected from the group consisting of an a glucagon like peptide 1 (GLP-1) therapy, a gastrointestinal peptide, a glucagon receptor agonist or antagonist, a glucose-dependent insulinotropic polypeptide (GIP) receptor agonist or antagonist, a ghrelin antagonist or inverse agonist, xenin, a xenin analogue, a biguanide, a sulfonylurea, a meglitinide, a thiazolidinedione, a DPP-4 inhibitor, an alpha-glucosidase inhibitor, a sodium-dependent glucose transporter 2 (SGLT-2) inhibitor, an SGLT-1 inhibitor, a peroxisome proliferator-activated receptor (PPAR−)(alpha, gamma or alpha/gamma) agonist or modulator, amylin, an amylin analogue, a G-protein coupled receptor 119 (GPR119) agonist, a GPR40 agonist, a GPR120 agonist, a GPR142 agonist, a systemic or low-absorbable TGR5 agonist, a diabetes immunotherapeutic, an anti-inflammatory agents for the treatment of the metabolic syndrome and diabetes, an adenosine monophosphate-activated protein kinase (AMPK) stimulant, an inhibitor of 11-beta-hydroxysteroid dehydrogenase 1, an activator of glucokinase, an inhibitor of diacylglycerol O-acyltransferase (DGAT), a modulator of glucose transporter-4, a somatostatin receptor 3 agonist, a lipid lowering agent, and a combination thereof.

In one embodiment of said use and/or method the antibody or antigen-binding fragment thereof reduces the LDL-C level of the patient by at least 30%, 35%, 40%, or 45%.

In one embodiment of said use and/or method the antibody or antigen-binding fragment thereof reduces the non-HDL-C level of the patient by at least 20%, 25%, 30%, or 35%.

In one embodiment of said use and/or method the antibody or antigen-binding fragment thereof reduces the ApoC3 level of the patient.

In one embodiment of said use and/or method the antibody or antigen-binding fragment thereof reduces the number and/or size of lipoprotein particles in the patient.

In one embodiment of said use and/or method the antibody or antigen-binding fragment thereof:

(a) does not affect the hemoglobin A1c (HbA1c) level of the patient; and/or
(b) does not affect the fasting plasma glucose (FPG) level of the patient.

In further embodiments the present invention relates to uses and/or methods for treating hypercholesterolemia in a patient with T2DM and ASCVD, the method comprising:

(a) selecting a high cardiovascular risk patient receiving insulin therapy that has

### (i) T2DM,

### (ii) ASCVD, and

(iii) hypercholesterolemia not adequately controlled by maximally tolerated statin therapy;
(b) administering every two weeks to the patient 75 mg of an antibody or an antigen-binding fragment thereof which specifically binds human proprotein convertase subtilisin/kexin type 9 (PCSK9); and
(c) administering to the patient one or more following doses of 75 mg of the antibody or antigen-binding fragment thereof about every two weeks if the LDL-C level in the patient is lower than 70 mg/dL, or administering one or more following doses of 150 mg of the antibody or antigen-binding fragment thereof about every two weeks if the LDL-C level in the patient is greater than or equal to 70 mg/dL,
wherein the antibody or antigen-binding fragment thereof comprises an HCVR having the amino acid sequence of SEQ ID NO: 1 and an LCVR having the amino acid sequence of SEQ ID NO: 6, and wherein the patient receives a concomitant insulin therapy.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Generation of Human Antibodies to Human PCSK9

Human anti-PCSK9 antibodies were generated as described in U.S. Pat. No. 8,062,640. The exemplary PCSK9 inhibitor used in the following Example is the human anti-PCSK9 antibody designated “mAb316P,” also known as “REGN727,” or “alirocumab.” mAb316P has the following amino acid sequence characteristics: a heavy chain comprising SEQ ID NO:5 and a light chain comprising SEQ ID NO:9; a heavy chain variable region (HCVR) comprising SEQ ID NO:1 and a light chain variable domain (LCVR) comprising SEQ ID NO:6; a heavy chain complementarity determining region 1 (HCDR1) comprising SEQ ID NO:2, a HCDR2 comprising SEQ ID NO:3, a HCDR3 comprising SEQ ID NO:4, a light chain complementarity determining region 1 (LCDR1) comprising SEQ ID NO:7, a LCDR2 comprising SEQ ID NO:8 and a LCDR3 comprising SEQ ID NO:10.

### Example 2: A Randomized, Double-Blind, Placebo-Controlled, Parallel Group Study to Evaluate the Efficacy and Safety of Alirocumab in Insulin Treated Patients with Type 1 or Type 2 Diabetes and with Hypercholesterolemia at High Cardiovascular Risk not Adequately Controlled on Maximally Tolerated LDL-C Lowering Therapy

### Introduction

More than 380 million people worldwide have diabetes, most of whom will die from cardiovascular disease (CVD). Compared to people without diabetes, those with diabetes are at higher risk of developing CVD, develop associated clinical complications and at an earlier age, and have shortened life expectancy by about 6 to 7 years. In addition to the high human cost of disease, CVD contributes greatly to the overall healthcare expenditure in these patients.

This study, named Odyssey DM-Insulin, included adult patients with Type 1 or Type 2 diabetes mellitus on insulin therapy with hypercholesterolemia at high cardiovascular (CV) risk that was not adequately controlled on a maximally tolerated dose of statin with or without other lipid modifying therapy (LMT).

### Study Objectives

The primary objectives of the study were: (a) to evaluate the efficacy of alirocumab in comparison with placebo in the reduction of calculated low-density lipoprotein cholesterol (LDL-C) after 24 weeks of treatment in high cardiovascular risk patients with diabetes treated with insulin and with hypercholesterolemia not adequately controlled on maximally tolerated LDL-C lowering therapy; and (b) to evaluate the safety and tolerability of alirocumab in patients with diabetes treated with insulin.

The secondary objectives of the study was to evaluate the efficacy of alirocumab in comparison to placebo on other lipid parameters at Weeks 12 and 24 (e.g., measured LDL-C, non-high-density lipoprotein cholesterol (non-HDL-C), apolipoprotein B (Apo B), total cholesterol (TC), lipoprotein a (Lp(a)), high-density lipoprotein cholesterol (HDL-C), triglyceride (TG) levels, triglyceride rich lipoproteins (TGRL), apolipoprotein A-1 (Apo A-1), apolipoprotein C3 (ApoC3), and LDL particle number and size.

### Study Design

This was a Phase 3b randomized, double-blind, placebo-controlled, multinational and multicenter study to assess the efficacy and safety of alirocumab administered by subcutaneous (SC) injection in insulin treated patients at high CV risk with Type 1 or Type 2 diabetes mellitus and with hypercholesterolemia not adequately controlled on maximally tolerated LDL-C lowering therapy. The study consisted of a screening period of up to 3 weeks, a double-blind treatment period of 24 weeks, and a safety observation period of 8 weeks after the end of the double-blind treatment period.

Patients, unless they were statin intolerant, were taking a stable, maximally tolerated dose of statin therapy with or without other lipid modifying therapies (LMT). Statin dose and dose regimen as well as dose and dose regimen of other lipid modifying treatment(s) (if applicable) was stable throughout the entire study duration including for 4 weeks prior to the screening period, during the screening period, and from screening to randomization. Patients were on a stable diet for glucose and lipid management throughout the entire study duration from screening to the Week 24 visit. Patients were receiving treatment for diabetes in accordance with local/regional standards of care.

Patients were stratified by diabetes type (i.e., Type 1 diabetes versus Type 2 diabetes). Recruitment of patients with Type 2 diabetes was completed when approximately 400 patients had been randomized. Recruitment of patients with Type 1 diabetes was completed at the end of the targeted recruitment period.

Alirocumab was administered subcutaneously with a starting dose of 75 mg Q2W for 12 weeks with a blinded up-titration to alirocumab 150 mg Q2W at Week 12 if the LDL-C at the Week 8 visit was 70 mg/dL (1.81 mmol/L). Patients who have an LDL-C<70 mg/dL (1.81 mmol/L) at the Week 8 visit continued with alirocumab 75 mg Q2W until the end of the treatment period.

The data on lipid parameters from blood samples were masked after randomization. No attempts were made by the Investigator or patient to have the patient's lipid values independently evaluated after randomization until after the Week 24 visit, except for the safety of the patient, as per the Investigator's judgment.

Patients visited the study site at Weeks −3, 0, 8, 12, 20, and 24 with lab work at each visit. In addition, a phone visit was taken at Weeks 4 and 32.

Adverse events (AEs) that had occurred within 70 days of the last dose of investigational medicinal product (IMP) were documented. Patients with a serious adverse event (SAE) or an adverse event of special interest (AESI) were followed until resolution, stabilization, or death.

### Patient Selection

The study enrolled a total of 517 patients, including 76 patients with T1 DM and 441 patients with T2DM.

### Inclusion Criteria

The patients enrolled in this study satisfied all of the following criteria:

(1) Patient having Type 1 or Type 2 diabetes treated with insulin, and with the levels of LDL-C≥70 mg/dL (1.81 mmol/L) not adequately controlled by a stable, maximum dose/regimen of statin that was tolerated by the patient for at least 4 weeks prior to the screening visit (Week −3) with or without other LMT. The maximum dose/regimen of statin that was tolerated by the patient was the highest registered dose/regimen tolerated by the patient based on the Investigator's judgment or concerns. Some examples of acceptable reasons for a patient taking a lower statin dose included, but were not limited to, adverse effects on higher doses, advanced age, low body mass index (BMI), regional practices, local prescribing information, or concomitant medications. Patients may have been on an alternate day dose of statin as long as the dose is consistently taken (e.g., dose every Monday, Wednesday, Friday, etc). Concomitant treatment with more than 1 statin was not permitted. Patients who had documented statin intolerance, as judged by the Investigator, and who were no longer on statin therapy as a result were also eligible for the study. The reason(s) for not being on a maximum dose/regimen of statin (including statin intolerance) were documented in the case report form.

(2) Patients ≥18 years of age or legal age of majority at screening visit, whichever was greater.

(3) Patients diagnosed with Type 1 or Type 2 diabetes at least one year prior to the screening visit (Week −3). Patients diagnosed with Type 1 diabetes needed to meet all of the following criteria:

(a) diagnosis prior to the age of 30 years;
(b) treated with a multiple daily injection regimen/basal-prandial insulin regimen or insulin pump regimen within 6 months after diagnosis; and
(c) C-peptide <0.2 pmol/mL at the screening visit.

(4) Glycosylated hemoglobin (HbA1c)<10% at the screening visit (Week −3). Patients with an elevated HbA1c (up to 10%) were eligible provided that there was no plan to target a lower HbA1c during the study, based on the judgment of the Investigator.

(5) Patients with documented history of CVD (including CHD and/or CHD risk equivalents) and/or at least one additional CV risk factor.

History of CHD included at least one of the following:

(a) acute myocardial infarction (MI);
(b) silent MI;
(c) unstable angina;
(d) coronary revascularization procedure (e.g., percutaneuous coronary intervention (PCI) or coronary artery bypass graft surgery (CABG)); and
(e) clinically significant CHD diagnosed by invasive or noninvasive testing (such as coronary angiography, stress test using treadmill, stress echocardiography, or nuclear imaging).

CHD risk equivalents included at least one of the following:

(a) documented peripheral arterial disease satisfying at least one of the following criteria:
(i) current intermittent claudication (muscle discomfort in the lower limb that is both reproducible and produced by exercise and relieved by rest within 10 minutes) of presumed atherosclerotic origin together with ankle-brachial index 0.90 in either leg at rest;
(ii) history of intermittent claudication (muscle discomfort in the lower limb that is both reproducible and produced by exercise and relieved by rest within 10 minutes) together with endovascular procedure or surgical intervention in one or both legs because of atherosclerotic disease; and
(iii) history of critical limb ischemia together with thrombolysis, endovascular procedure or surgical intervention in one or both legs because of atherosclerotic disease; and
   (b) documented previous ischemic stroke with a focal ischemic neurological deficit that persisted more than 24 hours, considered as being of atherothrombotic origin. Computed tomography or magnetic radio imaging must have been performed to rule out hemorrhage and non-ischemic neurological disease.

Cardiovascular risk factors included at least one of the following:

(a) hypertension (established on antihypertensive medicine);
(b) current cigarette smoker;
(c) age ≥45 years for men and 55 years for women;
(d) history of micro/macroalbuminuria;
(e) history of diabetic retinopathy (preproliferative or proliferative);
(f) family history of premature CHD (in father or brother before 55 years of age; in mother or sister before 65 years of age);
(g) low HDL-C (male <40 mg/dL (1.0 mmol/L) and female <50 mg/dL (1.3 mmol/L)); and
(h) documented chronic kidney disease (CKD) as defined by 15 eGFR <60 mL/min/1.73 m² for 3 months or more, including the screening visit).

(6) Signed written informed consent.

### Exclusion Criteria

Patients who met all the above inclusion criteria were screened for the following exclusion criteria:

(1) Exclusion criteria related to study methodology:

(a) planned to initiate new LMT during the course of the study or to modify the dose of the current LMT;
(b) not on a stable dose of LMT (including statin or other LMT) for at least 4 weeks prior to the screening visit (Week −3) or from screening to randomization, unless statin intolerant, in which case there was no statin for 4 weeks prior to the screening visit/during the screening period;
(c) use of nutraceutical products or over-the-counter therapies that may affect lipids which have not been at a stable dose for at least 4 weeks prior to the screening visit (Week −3) or between screening and randomization visits;
(d) use of red yeast rice products within 4 weeks of the screening visit (Week −3) or between screening and randomization visits;
(e) use of systemic corticosteroids, unless used as replacement therapy for pituitary/adrenal disease with a stable regimen for at least 6 weeks prior to randomization. Topical, intra-articular, nasal, inhaled and ophthalmic steroid therapies were not considered as “systemic” and were allowed;
(f) use of continuous hormone replacement therapy unless the regimen has been stable in the past 6 weeks prior to the Screening visit (Week −3) and no plans to change the regimen during the study;
(g) recent (within 3 months prior to the screening visit (Week −3) or between screening and randomization visits) MI, unstable angina leading to hospitalization, uncontrolled cardiac arrhythmia, CABG, PCI, carotid surgery or stenting, stroke, transient ischemic attack (TIA), endovascular procedure or surgical intervention for peripheral vascular disease;
(h) planned to undergo scheduled PCI, CABG, carotid or peripheral revascularization during the study;
(i) history of New York Heart Association (NYHA) Class III or IV heart failure (see Table 1) within the past 12 months;
(j) systolic blood pressure >180 mmHg or diastolic blood pressure >110 mmHg at screening or randomization visit;
(k) patient who had received plasmapharesis treatment either within 2 months prior to the screening visit (Week −3), between screening and randomization, or who has plans to receive it;
(l) known history of hemorrhagic stroke;
(m) known history of loss of function of PCSK9 (i.e., genetic mutation or sequence variation) or known history of homozygous familial hypercholesterolemia;
(n) new cancer or active progression of cancer within the past 5 years, except for adequately treated basal cell skin cancer, squamous cell skin cancer, or in situ cervical cancer;
(o) known history of positive HIV test;
(p) patient who had taken any active investigational drugs within 1 month or 5 half-lives, whichever was longer;
(q) patients not previously instructed on a cholesterol lowering diet prior to the screening visit (Week −3);
(r) patient who withdrew consent during the screening (starting from signed ICF);
(s) unstable weight defined as a variation of >5 kg within 2 months prior to the screening visit, as judged by the Investigator;
(t) BMI >45 kg/m2 or plans to undergo bariatric surgery, weight loss program, or to initiate weight loss drugs during the course of the study;
(u) recent initiation of weight loss drugs (i.e., within 3 months prior to the screening visit or between screening and randomization) or recent bariatric surgery (within the last 6 months) and in an active weight loss phase, as judged by the Investigator;
(v) patients not treated with insulin for at least 6 months prior to the screening visit or not on a stable insulin regimen (i.e., a change in type of insulin, general timing/frequency of injections, mode or pattern of administration such as basal only (Type 2 diabetes), basal-prandial, etc.) for at least 3 months prior to the screening visit, or likelihood of requiring a change in insulin type/frequency or mode of injection during the study period;
(w) not on a stable insulin dose for at least 3 months prior to screening (i.e., more than a 30% variation in total daily insulin dose as judged by the Investigator), or likelihood of requiring intensification of insulin/antihyperglycemic regimen during the course of the study, as judged by the Investigator (e.g., addition of new agent, plans for titration of insulin dose, etc);
(x) other antihyperglycemic medications taken by the patient had not been stable for at least 3 months before the screening visit;
(y) history of recent decompensation of diabetes within 2 months prior to the screening visit (i.e., diabetic ketoacidosis or hyperosmolar hyperglycemic state (HHS));
(z) receiving or planned to receive renal replacement therapy during the study (e.g., hemodialysis, renal transplant, etc.);
(aa) presence of any clinically significant uncontrolled endocrine disease known to influence serum lipids or lipoproteins. Patients on thyroid replacement therapy could be included if the dosage of thyroxin had been stable for at least 3 months prior to screening and the patient's sensitive thyroid stimulating hormone (s-TSH) levels were within ±10% of the normal range of the laboratory at the screening visit;
(bb) laboratory findings during the screening period (not including randomization labs, except for pregnancy test):
(i) serum TG >400 mg/dL (4.52 mmol/L) (1 repeat lab is allowed);
(ii) positive serum or urine pregnancy test in women of childbearing potential;
(iii) positive test for Hepatitis B surface antigen or Hepatitis C antibody;
(iv) eGFR <15 mL/min/1.73 m2 according to 4-variable modification in diet of renal disease (MDRD) equation;
(v) ALT or AST >3×ULN (1 repeat lab is allowed); or
(vi) creatine Phosphokinase (CPK) >3×ULN (1 repeat lab is allowed); or
   (cc) conditions/situations such as:
(i) patients with short life expectancy;
(ii) requirement for concomitant treatment that could bias primary evaluation;
(iii) impossibility to meet specific protocol requirements (e.g., need for hospitalization, ability to make study visits, etc.);
(iv) patient was the Investigator or any Sub-Investigator, research assistant, pharmacist, study coordinator, other staff or relative thereof directly involved in the conduct of the protocol;
(v) uncooperative or any condition that could make the patient potentially non-compliant to the study procedures;
(vi) any technical/administrative reason that makes it impossible to randomize the patient in the study; or
(vii) any clinically significant abnormality identified at the time of screening that in the judgment of the Investigator or Sub-Investigator would preclude safe completion of the study or constrain endpoint assessments such as major systemic diseases, patients with short life expectancy.

TABLE 1
New York Heart Association (NYHA) functional
classification of heart failure

| Class | Patient Symptoms |
|---|---|
| Class I | No limitation of physical activity. Ordinary physical activ- |
| (Normal) | ity does not cause undue fatigue, palpitation, or dyspnea |
| | (shortness of breath). |
| Class II | Slight limitation of physical activity. Comfortable at rest, |
| (Mild) | but ordinaryphysical activity results in fatigue, palpitation, |
| | or dyspnea. |
| Class III | Marked limitation of physical activity. Comfortable at rest, |
| (Moderate) | but less than ordinary activity causes fatigue, palpitation, |
| | or dyspnea. |
| Class IV | Unable to carry out any physical activity without discom- |
| (Severe) | fort. Symptoms of cardiac insufficiency at rest. If any |
| | physical activity is undertaken, discomfort is increased. |

(2) Exclusion criteria related to the active comparator and/or mandatory background therapies: all contraindications to the background therapies or warning/precaution of use (when appropriate) as displayed in the respective National Product Labeling.

(3) Exclusion criteria related to the current knowledge of alirocumab:

(a) hypersensitivity to alirocumab or to any of the ingredients of alirocumab;
(b) pregnant or breastfeeding woman;
(c) woman of childbearing potential not protected by highly-effective method(s) of birth control (as defined in the ICF and/or in a local protocol addendum in case of specific local requirement) and/or who are unwilling or unable to be tested for pregnancy. Women of childbearing potential must have a confirmed negative pregnancy test at screening and inclusion visits. They must use an effective contraceptive method throughout the entire duration of the study treatment and for at least 10 weeks after the last injection of IMP. The applied methods of contraception had to meet the criteria for a highly effective method of birth control according to the “International Conference on Harmonisation of Technical Requirements for Registration of Pharmaceuticals for Human Use. M3(R2): Guidance on nonclinical safety studies for the conduct of human clinical trials and marketing authorization for pharmaceuticals. ICH. 2009 June: 1-25.” Postmenopausal women must be amenorrheic for at least 12 months.

### Study Treatments

### Investigational Medicinal Product

Sterile alirocumab drug product was supplied at a concentration of 75 mg/mL and 150 mg/mL in an aqueous buffer, pH 6.0, containing sucrose, histidine, and polysorbate 20, both as 1 mL volume, in an auto-injector (also known as prefilled pen). Sterile placebo for alirocumab was prepared in the same formulation as alirocumab without the addition of protein as 1 mL volume in a prefilled pen, for the patients to perform injection training, as well as for those in the placebo treatment arm. During the screening period, the patient (or another designated person) had to perform a placebo self-injection training, using a prefilled pen, before the first administration of IMP.

For the patients randomized to alirocumab, the initial dose was 75 mg administered subcutaneously once Q2W. The dose was increased in a blinded fashion to 150 mg Q2W at Week 12 for patients randomized to alirocumab if the Week 8 LDL-C value is 70 mg/dL (1.81 mmol/L). The patients randomized to placebo were administered their injection subcutaneously Q2W throughout the duration of the 24-week treatment period.

### Route and Method of Administration

A prefilled pen training guide (auto-injector training guide) was provided to the sites and instructions for use (auto-injector for use) were provided to the patient. Each administration of IMP consisted of 1 mL subcutaneous injection in the abdomen, thigh, or outer area of upper arm (i.e., deltoid region). If another concomitant drug was being injected at the same site planned for the IMP injection, then the patient was advised to use an alternate location for administration of the IMP.

The IMP could be administered by self-injection or by another designated person (such as a spouse, relative, etc). In case a designated person was due to inject alirocumab to a patient during the study, it was ensured that this person had been adequately trained prior to administering the injection. Anyone that planned to administer the IMP was trained by the study staff.

Instructions were provided to the patient (or another designated person (such as spouse, relative, etc.) that would administer the injections) at training and as needed during the course of the study. Close supervision and feedback was given at the first visit, and other visits as needed.

The used prefilled pen was discarded in a sharps container which was provided to patients. It was recommended that the subcutaneous IMP injections be rotated within an anatomical area (e.g., right thigh, then left thigh or right abdomen, then left abdomen). Patients also had the option to inject in a different anatomical area (e.g., thigh then abdomen or the outer area of upper arm, etc) during the study.

Patients were asked to store the IMP in a refrigerator. Prior to administration, the IMP should be set outside in a safe location at room temperature for about 30 to 40 minutes. Thereafter, the IMP should be administered as soon as possible.

### Timing of Administration

During the screening period, patients or the designated person had to perform a placebo self-injection training using a prefilled pen, before the first IMP injection.

At the randomization visit, the first IMP injection was done at the site by the patient or another designated person (such as spouse, relative, etc) under direct site staff supervision. Patients were monitored at the investigational site for at least 30 minutes after this first injection in this study. If the designated person changed during the course of the study, the new designated person was trained with placebo.

IMP subcutaneous injections were then performed outside of the clinic, Q2W up to the last injection. If the injection was scheduled to take place on the same date as the site visit, then the IMP was administered after the blood sampling had been completed. In exceptional cases, if a patient preferred to have the injection performed at the study site and provisions were able to be made to accommodate the administration of injections at the site, it was also allowed.

IMP should be administered subcutaneously Q2W, ideally at approximately the same time of the day. However, it was acceptable to have a window period of ±3 days. The time of the day was based upon the patient's preference.

If by mistake or due to other circumstances an injection was delayed by more than 7 days from the missed date or completely missed, then the patient was requested to return to the original schedule of IMP administration without administering delayed injections. If by mistake or due to other circumstances an injection was delayed by less than or equal to 7 days from the missed date, then the patient was requested to administer the delayed injection and then resume the original schedule of IMP administration.

### Noninvestigational Medications

The following classes of drugs were identified as non-IMP because the medication is either a background therapy or a potential rescue medication:

(a) statins;
(b) cholesterol absorption inhibitors (ezetimibe);
(c) bile acid-binding sequestrants (such as cholestyramine, colestipol, colesevelam);
(d) nicotinic acid;
(e) fibrates (such as Fenofibrate);
(f) omega-3 fatty acids (1000 mg daily); and
(g) insulins.

For background LMT, including statins, sites followed the national product label for the safety monitoring and management of patients. Patients were on stable, maximum dose/regimen of statin therapy that was tolerated by the patient with or without other LMT during the study. Lipid profile values were blinded from samples obtained after randomization. Nevertheless, for safety reasons, sites were made aware of TG alerts, in order to make decisions on the patient's background LMT.

From the screening visit (Week −3) until the Week 24 visit, the background LMT was not changed. No dose adjustment, discontinuation, or initiation of other statins or other LMT took place during this time, unless in exceptional circumstances whereby overriding concerns (including but not limited to a TG alert posted by the central lab) warranted such changes, as per the Investigator's judgment. For a TG alert that had been confirmed by repeat testing, the Investigator performed investigations, managed the patient, and modified the background LMT as per his/her medical judgment.

All fibrates were allowed at entry if the patient had tolerated the medication and remained on a stable dose. If the patient required the introduction of a fibrate during the course of the study (i.e., as rescue treatment in response to a TG alert), only fenofibrate was allowed to be added. Background LMT and insulin were provided by the Sponsor. Patients obtained these medications in compliance with local regulations.

Blinding Procedures

Alirocumab and placebo for alirocumab were provided in identically matched prefilled pens and packaged identically, which included labeling to protect the blind. Each treatment kit was labeled with a number which was generated by a computer program from the sponsor. The treatment kit numbers were obtained by the Investigator at the time of patient randomization and subsequent patient visits scheduled via IVRS/IWRS that were available 24 hours-a-day, 7 days-a-week.

In accordance with the double-blind design, study patients, Investigators and study site personnel remained blinded to study treatment and did not have access to the randomization (treatment codes) except under circumstances described below.

### Adverse Event

The treatment code was unblinded by the Pharmacovigilance Department for reporting to the Health Authority of any Suspected Unexpected Serious Adverse Reaction (SUSAR), i.e., any serious adverse event that was both unexpected (per the specific section of the CIB) and reasonably associated with the use of the IMP according to the judgment of the Investigator and/or the Sponsor.

### Lipid Parameters

Lipid parameter values from blood samples obtained after the randomization visit, run by the central lab, were not communicated to the sites so that they were not able to deduce the treatment group of their patients based on LDL-C level attained. The sponsor's operational team did not have access to lipid parameters associated with patient identification until after the final database lock had occurred. For safety purposes, TG alerts for TG values 500 mg/dL any time after randomization were sent to the Investigator.

At the end of the double-blind treatment period (Week 24 visit) the Investigator continued to manage the patient's lipids in accordance with standard practice. Any lipid values after randomization was redacted in the source documents and not shared with the Sponsor.

### Anti-Alirocumab Antibodies

Patient anti-alirocumab antibody results were not communicated to the sites while the study was ongoing. The sponsor's operational team did not have access to anti-alirocumab antibody results associated with a patient identification number until after the final database lock had occurred. The lab technicians involved in the determination of patient anti-alirocumab antibody titers were excluded from the operations team and a process was set up to prevent any potential unblinding.

### Randomization Code Breaking During the Study

In case of an AE, the code was broken in circumstances when knowledge of the IMP was required for treating the patient. If possible, contact was initiated with the Monitoring Team/Study Physician before breaking the code. All calls were documented by the Monitoring Team as appropriate to include date and time of the call, name of the person contacted within the Monitoring Team, patient ID, documentation of the request, and decision for unblinding or not.

Code breaking could be performed at any time by using the proper module of the interactive voice response system (IVRS)/interactive web response system (IWRS), depending on which system was used for the site, and/or by calling any other phone number provided by the Sponsor for that purpose. However, it was preferable to contact the Study Physician to discuss the case before unblinding the case. If the blind was broken, the Investigator was requested to document the date, time of day, and reason for code breaking, and report this information on the appropriate page of the e-CRF. When documenting the reason for unblinding, the Investigator did not provide any detail regarding the nature of the IMP. The Investigator did not divulge IMP detail to the Sponsor's representative or to any staff members until database closure. Furthermore, when completing forms (e.g., AE, SAE), the study treatment was not disclosed on the forms.

The code-breaking material was also kept at the entity responsible for the “24 hour alert system”; but this system should be used in very exceptional cases only (i.e., unavailability of IVR/IWR system or inability to contact Investigator and/or site staff). However, the preferred option was to unblind using IVRS. The Investigators were informed by the clinical monitoring team about the availability of the local code-breaking material. A patient card, including the relevant “24 hour alert system” telephone number, was provided to every patient who will participate in the study. Unblinding was also allowed to be performed by the Sponsor for some SAEs in order to conform to regulatory reporting requirements (i.e., for some SAEs that are both related and unexpected).

If the code was broken, the patient permanently discontinued IMP administration.

### Method of Assigning Patients to Treatment Group

The randomized list of treatment kit numbers was generated centrally by the sponsor. The IMP (alirocumab 75 or 150 mg kits, or placebo kit) was packaged in accordance with this list.

The Trial Supply Operations Manager provided the randomized list of treatment kit numbers, and the Study Biostatistician provided the randomization scheme to the centralized treatment allocation system provider. Then, this centralized treatment allocation system provider generated the patient randomization list according to which it allocated the treatments to the patients.

Patients were randomized to receive either placebo or alirocumab during the double-blind treatment period. The randomization ratio alirocumab:placebo was 2:1. For each randomized patient, there were several corresponding treatment kit numbers (resupply visits), which were allocated through the centralized treatment allocation system. The randomization was stratified by diabetes type (i.e., Type 1 versus Type 2).

The treatment kit numbers were allocated using the centralized treatment allocation system on randomization visit (Day 1, Week 0), and then at Week 12 as re-supply visits, and at unscheduled visits if needed.

For patients in the alirocumab treatment arm, the treatment kit allocated at Week 12 was based on their Week 8 LDL-C level following the up-titration rules. Regular transfer of data was planned between the central laboratory and the centralized treatment allocation system provider in order to proceed in a blinded manner for study sites and sponsor.

Before randomizing a patient, the Investigator or designee had to contact the centralized treatment allocation system.

A randomized patient was defined as a patient who was registered and assigned with a treatment kit number from the centralized treatment allocation system, as documented from its log file. A patient could not be randomized more than once in the study. If a treatment was used without contacting the centralized treatment allocation system, then the patient was considered as not randomized and withdrawn from the study.

Two types of centralized treatment allocation systems, the IVRS and the IWRS, were used depending on the choice of the site.

### Packaging and Labeling

For the double-blind treatment period, each double-blind treatment kit, either alirocumab or placebo for alirocumab, was prepared to contain 6 prefilled pens in a child resistant package. In order to protect the blind, all double-blind treatment kit boxes for injection had the same look and feel and therefore will be labeled with a double-blind label.

In addition to the double-blind treatment kits for injection, a training kit containing 1 placebo for alirocumab prefilled pen was prepared for the purpose of instructing patients on injection administration which was to be performed prior to randomization at screening visit (Week−3, Visit 1). If deemed necessary, a second injection training with placebo for alirocumab was performed using an additional training kit prior to randomization. Injection training with placebo was performed and documented in the CRF, including if the designated person who administered IMP to the patient changed during the course of the study.

Packaging was in accordance with the administration schedule. The content of the labeling was in accordance with the local regulatory specifications and requirements.

### Storage Conditions and Shelf Life

Investigators or other authorized persons (e.g., pharmacists) were responsible for storing the IMP in a secure and safe place in accordance with local regulations, labeling specifications, policies, and procedures. Control of IMP storage conditions, especially control of temperature (e.g., refrigerated storage) and information on in-use stability and instructions for handling the IMP, was managed according to the rules provided by the Sponsor.

The IMP was stored in a refrigerator between +2° C. and +8° C. (36° F. to 46° F.) at the site. The temperature of the site refrigerator was checked daily and recorded on a log sheet. The IMP that was stored at the investigational site was kept in an appropriate locked room, under the responsibility of the Investigator or designee or other authorized person in accordance with the storage conditions indicated on the label.

After the supply of IMP kits to patients at the study site visits, appropriate provisions were in place for transportation of the IMP kits from the study site to the patient's refrigerator.

### Study Endpoints

Baseline characteristics included standard demography (e.g., age, race, weight, height, etc.), disease characteristics including medical history, and medication history for each patient.

### Primary Efficacy Endpoint

The primary efficacy endpoint was the percent change in LDL-C from baseline to Week 24 in the intent-to-treat (ITT) population, using all LDL-C values regardless of adherence to treatment (ITT estimand). The percent change was defined as 100× (calculated LDL-C value at Week 24-calculated LDL-C value at baseline)/calculated LDL-C value at baseline.

The baseline calculated LDL-C value was the last LDL-C level obtained before the first double-blind IMP injection. The calculated LDL-C at Week 24 was the LDL-C level obtained within the Week 24 analysis window. All calculated LDL-C values (scheduled or unscheduled, fasting or not fasting) between Weeks 8 to 24 were allowed to be used to provide a value for the primary efficacy endpoint, if appropriate, according to above definition.

### Primary Safety Endpoints

Safety parameters (AEs, laboratory parameters, vital signs) were assessed throughout the study. The observation of safety data was as follows:

(a) pre-treatment period was defined from the signed informed consent up to the first dose of double-blind IMP injection;
(b) treatment emergent adverse event (TEAE) period was defined as the time from the first dose of double-blind IMP injection to the last dose of IMP injection+70 days (10 weeks) as residual effect of treatment is expected until 10 weeks after the stop of double-blind IMP; and
(c) post-treatment period was defined as the time starting the day after the end of the TEAE period up to resolution/stabilization of all SAE and AESI, whichever came last.

An AE was any untoward medical occurrence in a patient or clinical investigation patient administered a pharmaceutical product and which did not necessarily have to have a causal relationship with this treatment.

An SAE was any untoward medical occurrence that at any dose:

(a) resulted in death;
(b) was life-threatening. The term “life-threatening” in the definition of “serious” referred to an event in which the patient was at risk of death at the time of the event; it did not refer to an event which hypothetically might have caused death if it were more severe;
(c) required inpatient hospitalization or prolongation of existing hospitalization;
(d) resulted in persistent or significant disability/incapacity;
(e) was a congenital anomaly/birth defect; or
(f) was a medically important event.

Medical and scientific judgment should be exercised in deciding whether expedited reporting was appropriate in other situations, such as important medical events that may not be immediately life-threatening or result in death or hospitalization but may jeopardize the patient or may require medical or surgical intervention (i.e., specific measures or corrective treatment) to prevent one of the other outcomes listed in the definition above.

The following list of medically important events was intended to serve as a guideline for determining which condition had to be considered as a medically important event. The list was not intended to be exhaustive:

(a) intensive treatment in an emergency room or at home for allergic bronchospasm, blood dyscrasias (i.e., agranulocytosis, aplastic anemia, bone marrow aplasia, myelodysplasia, pancytopenia, etc), or convulsions (seizures, epilepsy, epileptic fit, absence, etc.);
(b) development of drug dependence or drug abuse;
(c) ALT >3×ULN+total bilirubin >2×ULN or asymptomatic ALT increase >10×ULN;
(d) suicide attempt or any event suggestive of suicidality;
(e) syncope, loss of consciousness (except if documented as a consequence of blood sampling);
(f) bullous cutaneous eruptions;
(g) cancers diagnosed during the study or aggravated during the study;
(h) chronic neurodegenerative diseases (newly diagnosed) or aggravated during the study; and
(i) suspected transmission of an infectious agent, if any suspected transmission of an infectious agent via a medicinal product (e.g., product contamination).

An adverse event of special interest (AESI) is an AE (serious or non-serious) that needs to be monitored, documented, and managed in a pre-specified manner. For this study, the AESI were:

(a) increase in ALT: ALT≥ULN (if baseline ALT <ULN) Or ALT ≥2 times the baseline value (if baseline ALT≥ULN);
(b) allergic events: allergic drug reactions and/or local injection site reactions deemed to be allergic by the Investigator (or have an allergic component), that required consultation with another physician for further evaluation of hypersensitivity/allergy as per the Investigator's medical judgment should be reported as an AESI;
(c) pregnancy: pregnancy occurring in a female patient or the partner of a male patient (if permitted by the female partner and by local regulatory authorities) during the study or within 70 days following the last dose of study drug. Pregnancy was recorded as AESI in all cases. Pregnancy was qualified as an SAE only if it fulfilled one or more SAE criteria. In the event of pregnancy of a female patient included in the study, study product was discontinued. The follow-up of the pregnancy was mandatory until the outcome has been determined;
(d) symptomatic overdose with IMP. An overdose (accidental or intentional) was an event suspected by the Investigator or spontaneously notified by the patient (not based on systematic injection counts) and defined as at least twice of the intended dose within the intended therapeutic interval (i.e., 2 or more injections are administered in <7 calendar days), to be reported using the term “symptomatic OVERDOSE (accidental or intentional), indicating the circumstance in parentheses (e.g., “symptomatic overdose (accidental)” or “symptomatic overdose (intentional)”). The patient was monitored and appropriate symptomatic treatment instituted. The circumstances of the overdose were clearly specified in the verbatim and symptoms, if any, entered on separate AE/SAE forms. Asymptomatic overdose was requested to be reported as a standard AE;
(e) neurologic events: neurologic events that require additional examinations/procedures and/or referral to a specialist were requested to be reported as an AESI. If the event did not require additional examinations/procedures and/or referral to a specialist, it was requested to be reported as a standard AE; and
(f) Neurocognitive events: all neurocognitive events were considered as AESI.

### Secondary Efficacy Endpoints

Key secondary endpoints of the present study were as follows:

(a) percent change in calculated LDL-C from baseline to Week 24, using all LDL-C values during the efficacy treatment period (on-treatment estimand);
(b) percent change in measured LDL-C from baseline to Week 24 (ITT estimand);
(c) percent change in calculated LDL-C from baseline to Week 12 (ITT estimand);
(d) percent change in measured LDL-C from baseline to Weeks 12 (ITT estimand);
(e) percent change in non-HDL-C from baseline to Week 24 (ITT estimand);
(f) percent change in Apo B from baseline to Week 24 (ITT estimand);
(g) percent change in total cholesterol from baseline to Week 24 (ITT estimand);
(h) the proportion of patients reaching LDL-C<70 mg/dL at Week 24 (on-treatment estimand);
(i) the proportion of patients reaching LDL-C<50 mg/dL at Week 24 (on-treatment estimand);
(j) the proportion of patients reaching non-HDL-C<100 mg/dL at Week 24 (on-treatment estimand);
(k) the proportion of patients reaching non-HDL-C<80 mg/dL at Week 24 (on-treatment estimand);
(l) the percent change in Lp(a) from baseline to Week 24 (ITT estimand);
(m) the percent change in HDL-C from baseline to Week 24 (ITT estimand);
(n) the percent change in TG from baseline to Week 24 (ITT estimand);
(o) the percent change in LDL-C particle number from baseline to Week 24 (ITT estimand); and
(p) the percent change in LDL-C particle size from baseline to Week 24 (ITT estimand).

The following diabetes-related endpoints were also measured in the study:

(a) absolute change in HbA1c from baseline to Weeks 12 and 24 (ITT and on-treatment estimands);
(b) absolute change in FPG from baseline to Weeks 12 and 24 (ITT and on-treatment estimands);
(c) absolute change in total daily insulin dose from baseline to Weeks 12 and 24 (ITT and on-treatment estimands); and
(d) absolute change in number of glucose-lowering treatments from baseline to Weeks 12 and 24 (ITT and on treatment estimands).

Other efficacy endpoints of this study included:

(a) percent change in calculated LDL-C from baseline to Week 12 (on-treatment estimand);
(b) percent change in measured LDL-C from baseline to Weeks 12 and 24 (on-treatment estimand);
(c) percent change non-HDL, Apo B, total cholesterol, Lp(a), HDL-C, and TG from baseline to Weeks 12 (ITT and on-treatment estimands) and Week 24 (on-treatment estimand);
(d) proportion of patients reaching calculated LDL-C<50 and also <70 mg/dL at Weeks 12 (ITT and on-treatment estimands) and 24 (ITT estimand);
(e) proportion of patients with 50% or greater reduction from baseline in calculated LDL-C at Weeks 12 and 24 (ITT estimand);
(f) proportion of patients reaching non-HDL-C<80 mg/dL and also <100 mg/dL at Weeks 12 (ITT and on-treatment estimands) and Week 24 (ITT estimand);
(g) proportion of patients reaching Apo B<80 mg/dL at Weeks 12 and 24 (ITT and on-treatment estimands);
(h) percent change in LDL-C particle number and size from baseline to Week 12 (ITT and on-treatment estimands) and Week 24 (on-treatment estimand);
(i) percent change in TGRL, Apo A-1, and Apo C-III from baseline to Weeks 12 and 24 (ITT and on treatment estimands);
(j) absolute change in ratio Apo B/Apo A-1 and TC/HDL-C from baseline to Weeks 12 and 24 (ITT and on-treatment estimands);
(k) proportion of patients reaching calculated LDL-C<70 and <50 mg/dL at Weeks 12 and 24 according to baseline A1c of <8% or ≥8% (ITT and on-treatment estimands); and
(l) proportion of patients reaching calculated LDL-C<70 mg/dL and <50 mg/dL at Weeks 12 and 24 according to baseline A1c of <median A1c or median A1c (ITT and on-treatment estimands).

### Study Procedures

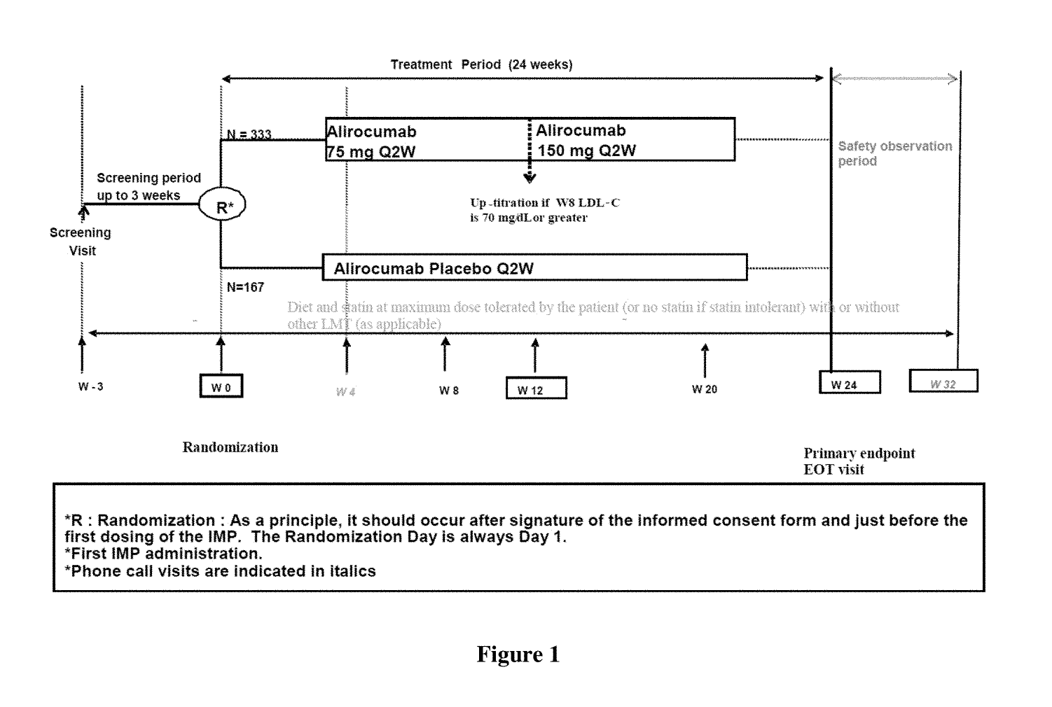

The window period for Week 0 was +3 days. The window period for Weeks 8, 12 and 24 was ±3 days. The window period for Weeks 4, 20, and 32 was ±7 days. For all visits after Day 1/inclusion visit, if one visit date was changed, then the next visit took place according to the original schedule as outlined in FIG. 1.

### Blood Sampling

All blood sampling, including the blood sampling for determination of lipid parameters (e.g., TC, LDL-C, HDL-C, TG, non-HDL-C, Apo A, Apo B, Apo C-III, Lp(a), LDL particle size and number) and also for plasma glucose, was performed in the morning, in fasting condition (i.e., overnight, at least 10 to 12 hours fast and refrain from smoking), and before IMP injection for all site visits throughout the study. Alcohol consumption within 48 hours and intense physical exercise within 24 hours preceding the blood sampling were discouraged. If the patient was not in fasting conditions, the blood sample was not collected, and a new appointment was given to the patient for the day after (or as close as possible to this date), with instructions to fast (see above conditions).

### Laboratory Tests

The laboratory data were collected in accordance with the study schedule outlined in FIG. 1 and the following guidelines:

(a) hematology: all visits except Weeks 4 and 20; may be performed at Week 0 as applicable and based on the clinical discretion of the Investigator;
(b) chemistry: all visits except Visits 3 and 6; may be performed at Week 0 as applicable and based on the clinical discretion of the Investigator, except for plasma glucose which should be performed at Week 0 for all patients;
(c) HbA1c: screening and Weeks 0, 12, and 24;
(d) lipid panel: screening and Weeks 0, 8, 12, 20, and 24;
(e) measured LDL-C via beta quantification: Weeks 0, 12, and 24;
(f) other lipid assessments (Apo B, Apo A-1, Apo C-III, LDL particle size and number, Lp[a]): Weeks 0, 12, and 24;
(g) liver panel: all visits except Visits 3 and 6; may be performed at Week 0 as applicable and based on the clinical discretion of the Investigator. In case of total bilirubin values above the normal range, differentiation into conjugated and non-conjugated bilirubin will occur automatically;
(h) creatine Phosphokinase (CPK): all visits except Visits 3 and 6; may be performed at Week 0 as applicable and based on the clinical discretion of the Investigator;
(i) Hepatitis B surface antigen: screening only;
(j) hepatitis C antibody: at screening and Week 24; in case of ALT increase during the study, hepatitis C antibody should be determined. If Hepatitis C antibody was positive during the study, reflexive testing was performed;
(k) pregnancy testing (in women of child bearing potential only): serum pregnancy test at screening only, urine pregnancy test at Weeks 0 and 24;
(l) thyroid stimulating hormone: screening only for patients who are taking thyroid hormone replacement;
(m) C-peptide: screening only;
(n) PCSK9 levels only at Week 0; and
(o) anti-alirocumab antibodies (Week 0, Week 12, and Week 24).

### Urine Samplings

Urinalysis was performed at screening and Week 24 visits. Dipstick was performed and assess for pH, specific gravity, and for the presence of blood, protein, glucose, ketones, nitrates, leukocyte esterase, uro-bilinogen and bilirubin. If the dipstick was abnormal, standard microscopy was then conducted. Microscopy was evaluate for the presence of red blood cells (RBC), RBC clumps, white blood cells (WBC), WBC clumps, epithelial cells (transitional, renal tubular, and squamous), casts (hyaline, epithelial, WBC, RBC, granular, fatty, cellular, broad, waxy), crystals (triple phosphate, calcium oxalate, calcium phosphate, calcium carbonate, uric acid, amorphous, ammonium biurate, bilirubin, leucine, tyrosine, cystine), bacteria, yeast-budding, yeast-hyphae, trichomonas, oval fat body, fat, mucous, and sperm.

Spot urine testing was performed for albumin and creatinine to calculate the albumin:creatinine ratio at the screening and Week 24 visits. Any clinically relevant abnormal laboratory value was immediately rechecked for confirmation before making any decision for the concerned patient.

### Physical Examination

A general physical examination was performed. If a new clinically significant abnormality or worsening from baseline was detected after inclusion, then an AE was reported and the patient was considered for further clinical investigations and/or specialist consultation, as per the Investigator's medical judgment.

### Blood Pressure and Heart Rate

Blood pressure (BP) was measured in sitting position under standardized conditions, approximately at the same time of the day, on the same arm, with the same apparatus (after the patient has rested comfortably in sitting position for at least 5 minutes). Values were recorded in the e-CRF; both systolic BP and diastolic BP were recorded. At the first screening visit, BP was measured in both arms. The arm with the highest diastolic pressure was determined at this visit, and BP was measured on this arm throughout the study. This highest value was recorded in the e-CRF.

Heart rate was measured at the time of the measurement of BP.

### Body Weight and Height

Body weight was obtained with the patient wearing undergarments or very light clothing and no shoes, and with an empty bladder. The same scale was used throughout the study.

Height was measured, as self-reported heights were not acceptable.

iTAQ Questionnaire

The iTAQ was a patient reported outcome (PRO) measure to assess treatment acceptability over the 4-week period prior to the completion of the questionnaire. It was requested to be completed by the patient at the Weeks 8 and 24 visits.

### Insulin Log

Patients were instructed to complete the insulin log in order to record his/her daily insulin dose (for basal insulin and for prandial insulin, as applicable) for at least 7 days prior to each visit, and to bring this information to the next study visit. The patient may record the daily insulin dose for more than 7 days prior to the study visits, however only the information collected for the last 7 days prior to each visit was entered into the CRF.

### Results

A total of 76 patients with T1 DM and 441 patients with T2DM were enrolled.

All 76 patients randomized with T1 DM were treated and therefore included in the safety population. Two randomized patients with T1 DM (both in the alirocumab group) were not included in the intent-to-treat (ITT) population.

Out of 441 patients T2DM, 3 were not treated (1 in the alirocumab group and 2 in placebo group) and therefore not included in the safety population. Twelve randomized patients with T2DM (7 in the alirocumab group and 5 in the placebo group) were not included in the ITT population.

Patients were not included in the ITT population if there was no calculated LDL-C value available at baseline or within one of the analysis windows up to Week 24.

### Study Patients

Six (7.9%) patients with T1 DM discontinued the study treatment prematurely (3 [5.9%] in the alirocumab group (2 patients discontinued due to AE) and 3 [12.0%] in the placebo group (2 patients discontinued due to AE). All 3 patients in the alirocumab group also did not complete the study period, whereas in the placebo group, 2 patients also did not complete the study period and 1 patient remained in the study until the completion of the study period.

TABLE 2
Disposition of patients with T1DM as per IVRS

| | Placebo | | Alirocumab | | All | |
|---|---|---|---|---|---|---|
| | (N = 25) | | (N = 51) | | (N = 76) | |
| Randomized but not treated | 0 | | 0 | | 0 | |
| Randomized and treated | 25 | (100) | 51 | (100) | 76 | (100) |
| Completed the study treatment period | 22 | (88.0) | 48 | (94.1) | 70 | (92.1) |
| Did not complete the study treatment period | 3 | (12.0) | 3 | (5.9) | 6 | (7.9) |
| Completed the study period | 22 | (88.0) | 49 | (96.1) | 71 | (93.4) |
| Did not complete the study period | 3 | (12.0) | 2 | (3.9) | 5 | (6.6) |
| Reason for treatment discontinuation | | | | | | |
| Adverse event | 2 | (8.0) | 2 | (3.9) | 4 | (5.3) |
| Subject did not wish to continue | 1 | (4.0) | 1 | (2.0) | 2 | (2.6) |
| Reason for study discontinuation | | | | | | |
| Subject did not wish to continue | 3 | (12.0) | 2 | (3.9) | 5 | (6.6) |
| Status at last study contact | | | | | | |
| Alive | 25 | (100) | 51 | (100) | 76 | (100) |

| | | | |
|---|---|---|---|
| Dead | 0 | 0 | 0 |

Percentages are calculated using the number of patients randomized as denominator.

Thirty-nine (8.8%) patients with T2DM discontinued the study treatment prematurely (29 (9.9%) in the alirocumab group and 10 (6.8%) in the placebo group. Out of the 29 patients in the alirocumab group, 18 patients also did not complete the study and 11 patients remained in the study until the completion of the study period. In the placebo group, out of the 10 patients, 7 patients also did not complete the study period and 3 patients remained in the study until the completion of the study period.

TABLE 3
Disposition of patients with T2DM as per IVRS

| | Placebo | | Alirocumab | | All | |
|---|---|---|---|---|---|---|
| | (N = 147) | | (N = 294) | | (N = 441) | |
| Randomized but not treated | 2 | (1.4) | 1 | (0.3) | 3 | (0.7) |
| Reason for not treated | | | | | | |
| Other | 2 | (1.4) | 1 | (0.3) | 3 | (0.7) |
| Randomized and treated | 145 | (98.6) | 293 | (99.7) | 438 | (99.3) |
| Completed the study treatment period | 135 | (91.8) | 264 | (89.8) | 399 | (90.5) |
| Did not complete the study treatment period | 10 | (6.8) | 29 | (9.9) | 39 | (8.8) |
| Completed the study period | 138 | (93.9) | 275 | (93.5) | 413 | (93.7) |
| Did not complete the study period | 7 | (4.8) | 18 | (6.1) | 25 | (5.7) |
| Reason for treatment discontinuation | | | | | | |
| Adverse event | 2 | (1.4) | 15 | (5.1) | 17 | (3.9) |
| Subject did not wish to continue | 3 | (2.0) | 8 | (2.7) | 11 | (2.5) |
| Poor compliance to study protocol | 2 | (1.4) | 0 | | 2 | (0.5) |
| Death | 1 | (0.7) | 0 | | 1 | (0.2) |
| Other | 2 | (1.4) | 6 | (2.0) | 8 | (1.8) |
| Reason for study discontinuation | | | | | | |
| Adverse event | 0 | | 1 | (0.3) | 1 | (0.2) |
| Death | 1 | (0.7) | 0 | | 1 | (0.2) |
| Subject did not wish to continue | 4 | (2.7) | 13 | (4.4) | 17 | (3.9) |
| Lost to follow-up | 0 | | 1 | (0.3) | 1 | (0.2) |
| Poor compliance to study protocol | 2 | (1.4) | 1 | (0.3) | 3 | (0.7) |
| Other | 0 | | 2 | (0.7) | 2 | (0.5) |
| Status at last study contact | | | | | | |
| Alive | 146 | (99.3) | 294 | (100) | 440 | (99.8) |

| | | | | | |
|---|---|---|---|---|---|
| Dead | 1 | (0.7) | 0 | 1 | (0.2) |

Percentages are calculated using the number of patients randomized as denominator.

### Demographics and Baseline Characteristics

Baseline characteristics were generally similar in the alirocumab and placebo groups. 60.5% of randomized patients with T1 DM were male, whereas 54.2% of randomized patients with T2DM were male. Patients with T1 DM were younger with mean age of 56.1 (SD=9.5) than patients with T2DM with mean age of 64.0 (SD=9.1). Mean BMI for patients with T1 DM was 30.0 kg/m² (SD=5.9) and a mean BMI of 32.6 kg/m² (SD=5.06) was observed for patients with T2DM.

TABLE 4
Demographics and patient characteristics at baseline -
Patients with Type 1 Diabetes as per IVRS

| | Placebo | | Alirocumab | | All | |
|---|---|---|---|---|---|---|
| | (N = 25) | | (N = 51) | | (N = 76) | |
| Age (years) | | | | | | |
| Number | | | | | | |
| Mean (SD) | 58.5 | (7.8) | 54.9 | (10.1) | 56.1 | (9.5) |
| Age group (years) | | | | | | |
| [n(%)] | | | | | | |
| <65 | 19 | (76.0) | 42 | (82.4) | 61 | (80.3) |
| >=65 to <75 | 6 | (24.0) | 8 | (15.7) | 14 | (18.4) |
| >=75 | 0 | | 1 | (2.0) | 1 | (1.3) |
| Gender [n(%)] | | | | | | |
| Number | | | | | | |
| Male | 17 | (68.0) | 29 | (56.9) | 46 | (60.5) |
| Female | 8 | (32.0) | 22 | (43.1) | 30 | (39.5) |
| Weight (kg) | | | | | | |
| Mean (SD) | 86.98 | (18.41) | 87.93 | (18.47) | 87.62 | (18.33) |
| BMI (kg/m²) | | | | | | |
| Mean (SD) | 28.71 | (4.82) | 30.59 | (6.25) | 29.97 | (5.85) |
| Systolic blood | | | | | | |
| pressure (mmHg) | | | | | | |
| Mean (SD) | 126.5 | (15.5) | 129.9 | (14.5) | 128.8 | (14.8) |
| Diastolic blood | | | | | | |
| pressure (mmHg) | | | | | | |
| Mean (SD) | 69.5 | (8.5) | 74.7 | (9.8) | 73.0 | (9.7) |
| Heart rate (bpm) | | | | | | |
| Mean (SD) | 68.4 | (9.3) | 73.0 | (10.9) | 71.5 | (10.6) |

BMI: Body Mass Index.

TABLE 5
Demographics and patient characteristics at baseline -
Patients with Type 2 Diabetes as per IVRS

| | Placebo | | Alirocumab | | All | |
|---|---|---|---|---|---|---|
| | (N = 147) | | (N = 294) | | (N = 441) | |
| Age (years) | | | | | | |
| Number | 147 | | 294 | | 441 | |
| Mean (SD) | 64.0 | (9.4) | 63.9 | (8.9) | 64.0 | (9.1) |
| Age group (years) | | | | | | |
| [n(%)] | | | | | | |
| <65 | 73 | (49.7) | 143 | (48.6) | 216 | (49.0) |
| >=65 to <75 | 55 | (37.4) | 126 | (42.9) | 181 | (41.0) |
| >=75 | 19 | (12.9) | 25 | (8.5) | 44 | (10.0) |
| Gender [n(%)] | | | | | | |
| Number | 147 | | 294 | | 441 | |
| Male | 78 | (53.1) | 161 | (54.8) | 239 | (54.2) |
| Female | 69 | (46.9) | 133 | (45.2) | 202 | (45.8) |
| Weight (kg) | | | | | | |
| Mean (SD) | 92.58 | (20.80) | 92.45 | (18.38) | 92.49 | (19.20) |
| BMI (kg/m²) | | | | | | |
| Mean (SD) | 32.69 | (5.50) | 32.55 | (4.84) | 32.60 | (5.06) |
| Systolic blood | | | | | | |
| pressure (mmHg) | | | | | | |
| Mean (SD) | 132.4 | (15.5) | 131.7 | (16.1) | 131.9 | (15.9) |
| Diastolic blood | | | | | | |
| pressure (mmHg) | | | | | | |
| Mean (SD) | 75.1 | (8.2) | 76.0 | (9.2) | 75.7 | (8.8) |
| Heart rate (bpm) | | | | | | |
| Mean (SD) | 72.5 | (10.9) | 72.8 | (10.6) | 72.7 | (10.7) |

BMI: Body Mass Index.

Calculated LDL-C at baseline was higher in patients with T1 DM (mean=121.0 mg/dL, SD=51.2) than patient with T2DM (mean=110.4 mg/dL, SD=37.3). Triglycerides at baseline were lower in patients with T1 DM median (Q1:Q3)=102.0 mg/dL (76.5:135.0) than in patients with T2DM median (Q1:Q3)=147.0 mg/dL (105.0:212.0).

TABLE 6
Lipid parameters at baseline - Quantitative summary in conventional
units - Patients with Type 1 Diabetes as per IVRS

| | Placebo | | Alirocumab | | All | |
|---|---|---|---|---|---|---|
| | (N = 25) | | (N = 51) | | (N = 76) | |
| Calculated LDL-C | | | | | | |
| (mg/dL) | | | | | | |
| Mean (SD) | 110.2 | (31.2) | 126.4 | (58.2) | 121.0 | (51.2) |
| Measured LDL-C | | | | | | |
| (mg/dL) | | | | | | |
| Mean (SD) | 109.8 | (31.4) | 127.7 | (58.1) | 121.7 | (51.1) |
| Non-HDL-C (mg/dL) | | | | | | |
| Mean (SD) | 130.7 | (34.2) | 150.2 | (62.9) | 143.8 | (55.6) |
| Total Cholesterol | | | | | | |
| (mg/dL) | | | | | | |
| Mean (SD) | 195.2 | (36.0) | 205.1 | (65.1) | 201.9 | (57.1) |
| HDL-C (mg/dL) | | | | | | |
| Mean (SD) | 64.4 | (17.4) | 54.9 | (13.7) | 58.1 | (15.6) |
| Triglycerides (mg/dL) | | | | | | |
| Mean (SD) | 100.0 | (38.1) | 119.1 | (58.4) | 112.8 | (53.1) |
| Triglycerides Rich | | | | | | |
| Lipoprotein (mg/dL) | | | | | | |
| Mean (SD) | 20.9 | (7.6) | 23.7 | (11.4) | 22.8 | (10.4) |
| Lipoprotein-(a) | | | | | | |
| (mg/dL) | | | | | | |
| Mean (SD) | 24.8 | (29.2) | 22.8 | (23.0) | 23.5 | (25.1) |
| Apolipoprotein B | | | | | | |
| (mg/dL) | | | | | | |
| Mean (SD) | 87.0 | (21.0) | 99.7 | (35.6) | 95.4 | (31.8) |
| Apolipoprotein A1 | | | | | | |
| (mg/dL) | | | | | | |
| Mean (SD) | 166.3 | (23.8) | 152.3 | (26.4) | 157.0 | (26.2) |

With respect to other lipid parameters, T1 DM patients exhibited a percentage reduction from baseline in LDL-C particle number (LS Mean) of 40.7% at week 12 and 44.4% at week 24 and percentage reduction in LDL-C particle size of 2.3% at week 12 and 2.3% at week 24. ApoC3 was reduced in these patients by 6.9% at week 12 and 7.5% at week 24.

TABLE 7
Lipid parameters at baseline - Quantitative summary in conventional
units - Patients with Type 2 Diabetes as per IVRS

| | Placebo | | Alirocumab | | All | |
|---|---|---|---|---|---|---|
| | (N = 147) | | (N = 294) | | (N = 441) | |
| Calculated LDL-C | | | | | | |
| (mg/dL) | | | | | | |
| Mean (SD) | 109.6 | (39.1) | 110.8 | (36.5) | 110.4 | (37.3) |
| Measured LDL-C | | | | | | |
| (mg/dL) | | | | | | |
| Mean (SD) | 110.5 | (37.4) | 112.1 | (34.3) | 111.6 | (35.3) |
| Non-HDL-C (mg/dL) | | | | | | |
| Mean (SD) | 144.9 | (48.5) | 144.7 | (42.6) | 144.8 | (44.6) |
| Total Cholesterol | | | | | | |
| (mg/dL) | | | | | | |
| Mean (SD) | 189.9 | (47.6) | 190.2 | (42.4) | 190.1 | (44.1) |
| HDL-C (mg/dL) | | | | | | |
| Mean (SD) | 44.9 | (13.2) | 45.5 | (12.5) | 45.3 | (12.7) |
| Triglycerides (mg/dL) | | | | | | |
| Mean (SD) | 189.4 | (148.2) | 174.6 | (110.1) | 179.5 | (124.2) |
| Triglycerides Rich | | | | | | |
| Lipoprotein (mg/dL) | | | | | | |
| Mean (SD) | 34.6 | (27.4) | 33.2 | (20.5) | 33.7 | (23.0) |
| Lipoprotein-(a) | | | | | | |
| (mg/dL) | | | | | | |
| Mean (SD) | 29.7 | (37.6) | 38.9 | (49.6) | 35.8 | (46.1) |
| Apolipoprotein B | | | | | | |
| (mg/dL) | | | | | | |
| Mean (SD) | 96.2 | (26.8) | 97.0 | (24.7) | 96.7 | (25.4) |
| Apolipoprotein A1 | | | | | | |
| (mg/dL) | | | | | | |
| Mean (SD) | 141.7 | (23.8) | 141.6 | (22.8) | 141.6 | (23.1) |

With respect to other lipid parameters, T2DM patients exhibited a percentage reduction from baseline in LDL-C particle number (LS Mean) of 37.6% at week 12 and 38.3% at week 24 and percentage reduction in LDL-C particle size of 2.6% at week 12 and 2.8% at week 24. ApoC3 was reduced in these patients by 6.3% at week 12 and 5.8% at week 24.

In patients with T1 DM, the mean durations of diabetes and of insulin use were similar between treatment groups. The mean duration of diabetes was 34.92 years (SD=12.67) and the mean duration of insulin use was 34.81 years (SD=12.77). In patients with T2DM, the mean durations of diabetes and of insulin use were similar between treatment groups. The mean duration of diabetes was 16.75 years (SD=8.13) and the mean duration of insulin use was 8.01 years (SD=6.90) in T2DM.

The duration of hypercholesterolemia was generally similar between treatment groups and between patients with T1 DM and T2DM.

The proportion of patients with statin intolerance, as was reported by the investigator, was 31.6% in patients with T1 DM and 23.8% in patients with T2DM.

The proportion of patients receiving fibrates at randomization was 2.6% in patients with T1 DM and 8.8% in patients with T2DM.

The proportion of patients on cholesterol absorption inhibitors (including ezetimibe) at randomization was higher in the alirocumab group (13.6%) than in the placebo group (7.6%), especially in patients with T2DM: 45 patients (15.3%) versus 10 patients (6.8%).

Cardiovascular history and risk factors were generally similar between treatment groups. The following differences were observed between patients with T1 DM and T2DM:

(1) ASCVD was more frequent in patients with T2DM than in patients with T1 DM (40.1% versus 21.1%) with more frequent coronary heart disease (34.7% versus 15.8%) and stroke (8.2% versus 2.6%), and less frequent PAD (4.3% versus 9.2%) in T2DM versus T1 DM patients.
(2) Among patients without ASCVD, 56.7% of patients with T1 DM had target organ damage (microalbuminuria, macroalbuminuria) and/or CKD and/or retinopathy versus 39.4% of patients with T2DM. Still among patients without ASCVD, the following additional cardiovascular risk factors were observed:
(a) More frequently in T1 DM than in T2DM patients: currently smoker (20.0% versus 14.0%), pre-proliferative diabetic retinopathy (36.7% versus 12.9%) and proliferative diabetic retinopathy (20.0% versus 5.7%).
(b) Less frequently in T1 DM than in T2DM patients: Hypertension (55.0% versus 84.8%), microalbuminuria (10.0% versus 19.7%), low HDL-C (16.7% versus 28.0%).
   (3) The presence of 3 or more additional CV risk factors in patients without ASCVD was observed in 45% of patients with T1 DM and 55.7% of patient with T2DM.

Overall, T1 DM and T2DM patients were treated with high and moderate intensity statins in both treatment groups with a higher proportion of patients were treated with moderate intensity statins (58.9%). Overall, 59.0% of T1 DM and T2DM patients were only treated with statin.

TABLE 9
Background lipid modifying therapies at randomization

| | Placebo | | Alirocumab | | All | |
|---|---|---|---|---|---|---|
| | (N = 172) | | (N = 345) | | (N = 517) | |
| Any statin [n (%)] | 129 | (75.0) | 258 | (74.8) | 387 | (74.9) |
| Intensity of statin^{a,c} [n (%)] | | | | | | |
| Number | 127 | | 257 | | 384 | |
| High | 38 | (29.5) | 102 | (39.5) | 140 | (36.2) |
| Moderate | 85 | (65.9) | 143 | (55.4) | 228 | (58.9) |
| Low | 4 | (3.1) | 12 | (4.7) | 16 | (4.1) |
| Statin alone [n (%)] | 100 | (58.1) | 205 | (59.4) | 305 | (59.0) |
| Any statin in addition to other LMT [n (%)] | 29 | (16.9) | 53 | (15.4) | 82 | (15.9) |
| Other LMT only (without statin) [n (%)] | 11 | (6.4) | 32 | (9.3) | 43 | (8.3) |
| Any LMT other than statins^{b} [n (%)] | 40 | (23.3) | 85 | (24.6) | 125 | (24.2) |
| Fibrates | 15 | (8.7) | 26 | (7.5) | 41 | (7.9) |
| Bile acid sequestrant | 0 | | 1 | (0.3) | 1 | (0.2) |
| Cholesterol absorption inhibitor | 13 | (7.6) | 47 | (13.6) | 60 | (11.6) |
| Nicotinic acid and derivates | 2 | (1.2) | 3 | (0.9) | 5 | (1.0) |
| Omega 3 fatty acids >= 1000 mg/day | 17 | (9.9) | 16 | (4.6) | 33 | (6.4) |
| PCSK9 inhibitor | 0 | | 0 | | 0 | |
| Nutraceuticals impacting lipids/other | 4 | (2.3) | 10 | (2.9) | 14 | (2.7) |
| No LMT (neither statins or other LMT) [n (%)] | 32 | (18.6) | 55 | (15.9) | 87 | (16.8) |

Note:
^{a}Only for patients who are currently taking statin.
^{b}in combination with statins or not.
^{c}High intensity statin corresponds to atorvastatin 40 to 80 mg daily or rosuvastatin 20 to 40 mg daily or simvastatin 80 mg daily.
Moderate intensity statin corresponds to atorvastatin 10 to 20 mg daily or rosuvastatin 5 to 10 mg daily or simvastatin 20 to 40 mg daily or pravastatin 40 to 80 mg daily or lovastatin 40 mg daily or fluvastatin 80 mg daily or pitavastatin 2 to 4 mg daily.
Low intensity statin corresponds to simvastatin 10 mg daily or pravastatin 10 to 20 mg daily or lovastatin 20 mg daily or fluvastatin 20 to 40 mg daily or pitavastatin 1 mg daily.
Patients receiving more than one intensity level of statin is counted in the highest intensity level.
% calculated using the number of patients randomized as denominator except for each intensity of statins where % is calculated using the number of patients on statins as denominator and for each daily dose category where % is calculated using the number of patients taking that particular statin as denominator.

The exposure of the safety population to investigational medicinal product is summarized in Table 10.

TABLE 10
Exposure to investigational medicinal product - Injection

| | Placebo | | Alirocumab | |
|---|---|---|---|---|
| | (N = 170) | | (N = 344) | |
| Duration of IMP injection | | | | |
| exposure (weeks) | | | | |
| Mean (SD) | 23.31 | (3.92) | 23.16 | (3.90) |
| Duration of IMP injection | | | | |
| exposure by category [n (%)] | | | | |
| >=1 day to <4 weeks | 0 | | 2 | (0.6) |
| >=4 weeks to <8 weeks | 5 | (2.9) | 6 | (1.7) |
| >=8 weeks to <12 weeks | 1 | (0.6) | 6 | (1.7) |
| >=12 weeks to <16 weeks | 3 | (1.8) | 7 | (2.0) |
| >=16 weeks to <24 weeks | 13 | (7.6) | 34 | (9.9) |
| >=24 weeks | 148 | (87.1) | 289 | (84.0) |
| Number of IMP injections | | | | |
| Mean (SD) | 11.3 | (2.0) | 11.3 | (2.1) |
| Titration [n (%)] | | | | |

| | | | |
|---|---|---|---|
| Patients up-titrated^{a} | NA | 77/326 | (23.6) |

Note:
^{a}up-titrated patients according to IVRS Week 12 transaction with at least one injection of alirocumab 150 mg afterwards. Denominator corresponding to patients with at least one injection post W12 IVRS transaction. Patients are considered in the treatment group they actually received.
The duration of IMP injection exposure in weeks is defined as: (last IMP injection date + 14 days − first IMP injection date)/7, regardless of intermittent discontinuations.

### Efficacy

### Primary Efficacy Endpoint

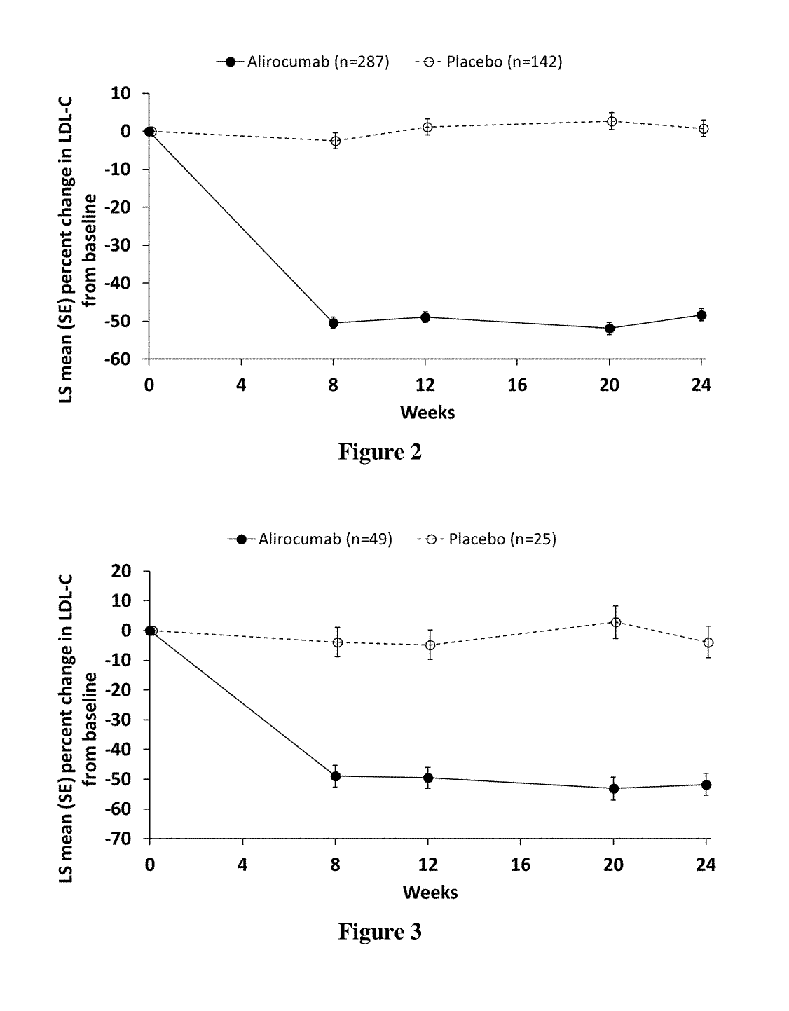

Alirocumab was superior to placebo on percentage of change in calculated LDL-C from baseline to Week 24 in the ITT population of patients with T1 DM and T2DM (as shown in Tables 11 and 12, and in FIGS. 2 and 3). Among the T1 DM patients, the proportion of individuals achieving LDL-C<70 mg/dL (<1.8 mmol/L) was 70.2% in the alirocumab group and 5.1% in the placebo group (P<0.0001), and the proportion of individuals attaining LDL-C of <50 mg/dL (1.3 mmol/L) was 55.1% in the alirocumab group and 0% in the placebo group (P value not computable). Among the T2DM patients, the proportion of individuals achieving LDL-C<70 mg/dL (<1.8 mmol/L) was 76.4% in the alirocumab group and 7.4% in the placebo group (P<0.0001), and the proportion of individuals attaining LDL-C of <50 mg/dL (1.3 mmol/L) was 50.7% in the alirocumab group and 2.4% in the placebo group (P<0.0001). The sensitivity analysis on the primary efficacy endpoint showed similar results in both populations (data are not shown).

TABLE 11
Percent change in calculated LDL-C from baseline to Week
24: MMRM - Patients with Type 1 Diabetes as per IVRS

| | Placebo | Alirocumab |
|---|---|---|
| Calculated LDL Cholesterol | (N = 25) | (N = 49) |
| Baseline (mmol/L) | | |
| Number | 25 | 49 |
| Mean (SD) | 2.9 (0.8) | 3.2 (1.2) |
| Median | 2.6 | 3.0 |
| Min:Max | 2:5 | 1:7 |
| Baseline (mg/dL) | | |
| Number | 25 | 49 |
| Mean (SD) | 110.2 (31.2) | 122.5 (47.8) |
| Median | 100.0 | 114.0 |
| Min:Max | 71:180 | 48:273 |
| Week 24 percent change | | |
| from baseline (%) | | |
| LS mean (SE) | −3.9 (5.3) | −51.8 (3.7) |
| LS mean difference (SE) | | −47.8 (6.5) |
| vs placebo | | |
| 95% CI | | (−60.7 to −35.0) |
| p-value vs placebo | | <.0001* |

Note:
Least-squares (LS) means, standard errors (SE) and p-value taken from MMRM (mixed-effect model with repeated measures) analysis.
The model includes the fixed categorical effects of treatment group, randomization strata as per IVRS, time point, and the interactions treatment group-by-time point, strata-by-time point, treatment group-by-strata and treatment group-by-strata-by-time point, as well as the continuous fixed covariates of baseline calculated LDL-C value and baseline value-by-time point interaction.
MMRM model run on all patients in the ITT population (i.e. Type 1 and Type 2 Diabetes patients).
MMRM model and baseline description run on patients with a baseline value and a post-baseline value in at least one of the analysis windows used in the model.
The p-value is followed by a ‘*’ if statistically significant according to the fixed hierarchical approach used to ensure a strong control of the overall type-I error rate at the 0.05 level.

TABLE 12
Percent change in calculated LDL-C from baseline to Week
24: MMRM - Patients with Type 2 Diabetes as per IVRS

| | Placebo | Alirocumab |
|---|---|---|
| Calculated LDL Cholesterol | (N = 142) | (N = 297) |
| Baseline (mmol/L) | | |
| Number | 142 | 287 |
| Mean (SD) | 2.8 (1.0) | 2.9 (0.9) |
| Median | 2.6 | 2.7 |
| Min:Max | 1:8 | 1:7 |
| Baseline (mg/dL) | | |
| Number | 142 | 287 |
| Mean (SD) | 109.5 (38.7) | 110.3 (35.9) |
| Median | 102.0 | 104.0 |
| Min:Max | 43:309 | 45:279 |
| Week 24 percent change | | |
| from baseline (%) | | |
| LS mean (SE) | 0.8 (2.2) | −48.2 (1.6) |
| LS mean difference (SE) | | −49.0 (2.7) |
| vs placebo | | |
| 95% CI | | (−54.4 to −43.6) |
| p-value vs placebo | | <.0001* |

Note:
Least-squares (LS) means, standard errors (SE) and p-value taken from MMRM (mixed-effect model with repeated measures) analysis.
The model includes the fixed categorical effects of treatment group, randomization strata as per IVRS, time point, and the interactions treatment group-by-time point, strata-by-time point, treatment group-by-strata and treatment group-by-strata-by-time point, as well as the continuous fixed covariates of baseline calculated LDL-C value and baseline value-by-time point interaction.
MMRM model run on all patients in the ITT population (i.e. Type 1 and Type 2 Diabetes patients).
MMRM model and baseline description run on patients with a baseline value and a post-baseline value in at least one of the analysis windows used in the model.
The p-value is followed by a ‘*’ if statistically significant according to the fixed hierarchical approach used to ensure a strong control of the overall type-I error rate at the 0.05 level.

### Secondary Efficacy Endpoints

Alirocumab resulted in significant reductions from baseline to Week 24 (difference versus placebo) in levels of non-HDL-C, ApoB, total cholesterol, and Lp(a), as well as increase in HDL-C in the ITT population of patients with T1DM and T2DM (as shown in Tables 13 and 14, respectively).

TABLE 13
Percent change in key secondary efficacy endpoints
from baseline - Patients with Type 1 Diabetes

| Selected key secondary | | | | | |
|---|---|---|---|---|---|
| efficacy endpoints | | | | | Difference vs. |
| % change from baseline, | Placebo | | Alirocumab | | placebo, % |
| mean (SD) | (N = 25) | | (N = 49) | | [95% CI], P-value |
| Calculated LDL-C | −4.5 | (5.0) | −49.4 | (3.5) | −44.8 (6.1) |
| (Week 12) | | | | | [−56.9 to −32.8], |
| | | | | | P < 0.0001 |
| Non-HDL-C | −3.2 | (4.8) | −45.9 | (3.3) | −42.7 (5.8) |
| (Week 24) | | | | | [−54.2 to −31.3], |
| | | | | | P < 0.0001 |
| Apo B (Week 24) | −0.4 | (4.3) | −39.4 | (3.0) | −39.0 (5.3) |
| | | | | | [−49.4 to −28.7], |
| | | | | | P < 0.0001 |
| Total cholesterol | −0.7 | (3.6) | −29.9 | (2.5) | −29.2 (4.3) |
| (Week 24) | | | | | [−37.8 to −20.7], |
| | | | | | P < 0.0001 |
| Lp(a) (Week 24) | −4.3 | (5.3) | −23.0 | (3.8) | −18.7 (6.5) |
| | | | | | [−31.4 to −6.0], |
| | | | | | P < 0.0001 |
| HDL-C (Week 24)† | 7.3 | (3.5) | 11.2 | (2.4) | 3.9 (4.1) |
| | | | | | [−4.2 to 12.0], |
| | | | | | P = 0.343‡ |
| Triglycerides | 1.9 | (6.7) | −13.6 | (4.7) | −15.5 (8.1) |
| (Week 24)† | | | | | [−31.4 to 0.4], |
| | | | | | P = 0.056‡ |
| LDL particle | −4.4 | (4.6) | −44.4 | (3.2) | −40.0 (5.6) |
| numbers (Week 24) | | | | | [−51.0 to −28.9], |
| | | | | | P < 0.0001‡ |
| LDL particle | 0.8 | (0.5) | −2.3 | (0.3) | −3.0 (0.6) |
| size (Week 24) | | | | | [−4.2 to −1.9], |
| | | | | | P < 0.0001‡ |
| ApoC3 (Week 24, ITT) | 1.6 | (5.8) | −7.4 | (4.0) | −9.0 |
| | | | | | [22.7 to 4.8] |
| ApoA-I (Week 24, ITT) | 7.9 | (2.6) | 10.2 | (1.8) | 2.3 |
| | | | | | [3.8 to 8.4] |
| TRL-C (Week 24, ITT) | −8.4 | (8.1) | −24.7 | (5.6) | −16.3 |
| | | | | | [−35.4 to 2.8] |
| TRL-C (Week 24, | −4.2 | (8.2) | −24.3 | (5.8) | −20.1 |
| on-treatment) | | | | | [39.5 to 0.6] |

†Hierarchical testing terminated at the endpoint triglycerides in participants with T2D and at the endpoint HDL-C in participants with T1D, therefore all subsequent statistical comparisons were not considered statistically significant.
‡P-values for descriptive purposes only.
Apo, apolipoprotein; ITT, intention-to-treat; LDL-C, low-density lipoprotein cholesterol; Lp(a), lipoprotein (a); LS, least squares; non-HDL-C, non-high-density lipoprotein cholesterol; TRL-C, triglyceride-rich lipoprotein cholesterol; SE, standard error; SD, standard deviation.

TABLE 14
Percent change in key secondary efficacy endpoints
from baseline - Patients with Type 2 Diabetes

| Selected key secondary | | | | | |
|---|---|---|---|---|---|
| efficacy endpoints | | | | | Difference vs. |
| % change from baseline, | Placebo | | Alirocumab | | placebo, % |
| mean (SD) | (N = 142) | | (N = 287) | | [95% CI], P-value |
| Calculated LDL-C | 1.4 | (2.1) | −48.8 | (1.4) | −50.2 (2.5) |
| (Week 12) | | | | | [55.2 to −45.3], |
| | | | | | P < 0.0001 |
| Non-HDL-C | 0.7 | (2.0) | −37.9 | (1.4) | −38.7 (2.4) |
| (Week 24) | | | | | [43.4 to −33.9], |
| | | | | | P < 0.0001 |
| Apo B (Week 24) | 3.3 | (1.7) | −33.4 | (1.3) | −36.7 (2.1) |
| | | | | | [40.9 to −32.5], |
| | | | | | P < 0.0001 |
| Total cholesterol | 0.8 | (1.5) | −26.8 | (1.0) | −27.6 (1.8) |
| (Week 24) | | | | | [31.2 to −24.1], |
| | | | | | P < 0.0001 |
| Lp(a) (Week 24) | −0.5 | (2.2) | −19.0 | (1.6) | −18.4 (2.7) |
| | | | | | [23.7 to −13.2], |
| | | | | | P < 0.0001 |
| HDL-C (Week 24)† | 3.7 | (1.4) | 8.1 | (1.0) | 4.4 (1.7) |
| | | | | | [1.1 to 7.7], |
| | | | | | P < 0.01 |
| Triglycerides | 0.0 | (2.7) | −5.7 | (2.0) | −5.7 (3.4) |
| (Week 24)† | | | | | [12.3 to 0.9], |
| | | | | | P = 0.0902‡ |
| LDL particle | 1.9 | (1.9) | −38.3 | (1.3) | −40.2 (2.3) |
| numbers (Week 24) | | | | | [44.7 to −35.6], |
| | | | | | P < 0.0001‡ |
| LDL particle | −0.3 | (0.2) | −2.8 | (0.1) | −2.5 (0.2) |
| size (Week 24) | | | | | [2.9 to 2.0], |
| | | | | | P < 0.0001‡ |
| ApoC3 (Week 24, ITT) | 4.2 | (2.3) | −5.8 | (1.7) | −10.0 |
| | | | | | [15.6 to 4.4] |
| ApoA-I (Week 24, ITT) | 4.5 | (1.0) | 7.4 | (0.7) | 2.9 |
| | | | | | [0.5 to 5.4] |
| TRL-C (Week 24, ITT) | 1.4 | (3.3) | −15.7 | (2.3) | −17.2 (4.0) |
| | | | | | [25.1 to −9.3] |
| TRL-C (Week 24, | 2.2 | (3.4) | −16.4 | (2.4) | −18.6 |
| on-treatment) | | | | | [26.6 to 10.5] |

†Hierarchical testing terminated at the endpoint triglycerides in participants with T2D and at the endpoint HDL-C in participants with T1D, therefore all subsequent statistical comparisons were not considered statistically significant.
‡P-values for descriptive purposes only.
Apo, apolipoprotein; ITT, intention-to-treat; LDL-C, low-density lipoprotein cholesterol; Lp(a), lipoprotein (a); LS, least squares; non-HDL-C, non-high-density lipoprotein cholesterol; TRL-C, triglyceride-rich lipoprotein cholesterol; SE, standard error; SD, standard deviation.

### Diabetes-Related Endpoints

Overall, FPG and HbA1c, and glucose lowering treatments remained stable over time in patients with T1 DM and T2DM in both treatment groups.

Regarding HbA1c, in the T1 DM cohort, in the alirocumab group the mean HbA1c % was 7.84% (SD=0.94) at baseline with mean absolute change=−0.03% (0.6), while in the placebo group the mean HbA1c was 7.68% (0.78) at baseline, with mean absolute change=−0.23% (0.36). In the T2DM cohort, in the alirocumab group the mean HbA1c was 7.52% (0.96) at baseline with mean absolute change=0.18% (0.74), while in the placebo group the mean HbA1c was 7.54% (1.02) at baseline with mean absolute change=0.06% (0.66).

Regarding FPG, in the T1 DM cohort, in the alirocumab group the mean FPG was 173 mg/dL (SD=70.6) at baseline with mean absolute change=−0.03 mg/dL (0.6), while in the placebo group the mean FPG was 166.5 mg/dL (75.6) at baseline with mean absolute change=14.6 mg/dL (75.9). In the T2DM cohort, in the alirocumab group the mean FPG was 154.1 mg/dL (50.1) at baseline with mean absolute change=9.5 mg/dL (61.8), while in the placebo group the mean FPG was 153.5 mg/dL (52.5) at baseline with mean absolute change=10.0 mg/dL (47.0).

### Safety

A total of 344 patients (51 T1 DM and 293 T2DM) were exposed to alirocumab and 170 patients (25 T1 DM and 145 T2DM) to a placebo.

Overall, rates of patients with any treatment emergent adverse events (TEAEs) were similar across treatment groups in the safety population of patients with T1 DM or T2DM (see Table 15).

TABLE 15
Overview of adverse event profile: treatment emergent adverse events

| | Placebo | | Alirocumab | | All | |
|---|---|---|---|---|---|---|
| | (N = 170) | | (N = 344) | | (N = 514) | |
| | n (%) | | n (%) | | n (%) | |
| Patients with any TEAE | 109 | (64.1) | 222 | (64.5) | 331 | (64.4) |
| Patients with any treatment emergent SAE | 14 | (8.2) | 25 | (7.3) | 39 | (7.6) |
| Patients with any TEAE leading to death | 1 | (0.6) | 0 | | 1 | (0.2) |
| Patients with any TEAE leading to | 4 | (2.4) | 17 | (4.9) | 21 | (4.1) |
| permanent treatment discontinuation | | | | | | |

Note:
TEAE: Treatment Emergent Adverse Event; SAE: Serious Adverse Event.
n (%) = number and percentage of patients with at least one TEAE.

TEAEs were more frequently 10%) reported in the following system organ classes (SOCs):

(a) infections and infestations (21.8% in alirocumab versus 21.8% in placebo);
(b) gastrointestinal disorders (13.1% in alirocumab versus 12.4% in placebo);
(c) musculoskeletal and connective tissue disorders (21.5% in alirocumab versus 15.9% in placebo); and
(d) general disorders and administration site conditions (11.0% in alirocumab versus 8.8% in placebo)

At the PT level, the most frequently reported TEAEs 2%) in the alirocumab group and with a difference ≥0.5% incidence from the placebo group were by decreasing order of frequency in the alirocumab group: myalgia (4.4% versus 1.8%), arthralgia (2.9% versus 1.8%), bronchitis (2.6% versus 0.6%), dizziness (2.6% versus 1.2%), and peripheral oedema (2.0% versus 0.6%). In contrast, the most frequently reported TEAEs (≥2%) in the placebo group and with a difference ≥0.5% incidence from the alirocumab group were: influenza (2.3% versus 2.9%), pain in extremity (1.7% versus 2.9%), hypoglycemia (1.7% versus 2.4%), cough (1.5% versus 2.9%), musculoskeletal pain (1.2% versus 2.4%), upper respiratory tract infection (0.9% versus 2.4%), hyperglycaemia (0.9% versus 2.4%), and pneumonia (0.6% versus 2.4%).

Overall, treatment emergent SAEs were reported in 25 patients (7.3%) in the alirocumab group and 14 patients (8.2%) patients in the placebo group. SAEs (at PT level) reported in more than 1 patient in either treatment groups were pneumonia (in 1 patient (0.3%) in the alirocumab group versus 2 patients (1.2%) in the placebo group), vertebral foraminal stenosis (in 2 patients (0.6%) in the alirocumab group versus no patients in the placebo group), and urinary tract infection (in 2 patients (0.6%) versus no patients in placebo group). One death due to myocardial infarction was reported in a T2DM patient of the placebo group, 1 month after the first IMP dose administration (Visit 3). Overall, 17 patients (4.9%) in the alirocumab group and 4 patients (2.4%) in the placebo group experienced TEAEs leading to permanent treatment discontinuation. At the PT level, the proportion of patients with TEAE leading to permanent treatment discontinuation in more than one patient of a treatment group were: headache (2 patients (0.6%) in the alirocumab group versus no patient in the placebo group), cognitive disorder (in 2 patients (0.6%) versus no patient), allergic dermatitis (2 patients (0.6%) versus no patient) and myalgia (3 patients (0.9%) versus 2 patient (1.2%)).

As to adverse events of special Interest (AESI), increase in ALT meeting AESI criteria were defined as ALT≥3×ULN (if baseline ALT <ULN) or ALT ≥2 times the baseline value (if baseline ALT≥ULN). These events were reported in 2 patients (0.6%) in the alirocumab group versus 1 patient (0.6%) in the placebo group.

Allergic drug reactions meeting AESI criteria were defined as allergic events that require consultation with another physician for further evaluation. These events were reported in 5 patients (1.5%) in the alirocumab group versus 4 patients (2.4%) in the placebo group. These reactions were mainly skin and subcutaneous tissue disorders, reported in 3 patients (0.9%) in the alirocumab group (1 allergic dermatitis, 1 eczema, and 1 photosensitivity reaction) and in 2 patients (1.2%) in the placebo group (1 dermatitis, 1 drug eruption). The two other AESI allergic drug reactions in the alirocumab group were drug hypersensitivity and eosinophilia.

Neurologic events meeting AESI criteria were defined as neurologic events that require additional examinations/procedures and/or referral to a specialist. Such events were reported in 1 patient (0.3%) in the alirocumab group (paraesthesia) versus 1 patient (0.6%) in the placebo group (dysphagia). Both events were reported in T2DM patients.

All neurocognitive events were considered as AESI. Neurocognitive events as per sponsor or FDA grouping were reported in 4 patients (1.2%) in the alirocumab group versus no patients in the placebo group. All events were reported in T2DM patients: a cognitive disorder was reported in 2 patients (0.6%), and memory impairment and amnesia in 1 patient (0.3%) each. Of note, the 2 cognitive disorders also led to permanent treatment discontinuation.

Local injection site reactions meeting AESI criteria were defined as either local injection site reactions that were allergic and required consultation with another physician or local injection site reactions that were non-allergic that were clinically significant (e.g. reaction of swelling or erythema with a diameter >2.5 cm; reaction that interferes with activity). LISR confirmed per investigator as related to IMP (‘per eCRF’) were reported in 6 patients (1.7%) in the alirocumab group versus 8 patients (4.7%) in the placebo group (injection site reaction of the placebo for alirocumab). No local injection site reactions (LISR) meeting AESI criteria, defined as reactions that require consultation with another physician for further evaluation, were reported.

There was no report of symptomatic overdose or pregnancy.

Analyses of liver function tests (ALT, AST, ALP, total bilirubin), CPK and renal function tests (creatinine, eGFR, BUN) did not reveal differences between the treatment groups in the changes over time for any of the studied parameters. PCSA analyses did not identify PCSA of ALT increase in any treatment groups during the study. In patients with normal CPK values at baseline, an increase >3 ULN (and 10 ULN) was reported in 7 patients (2.1%) in the alirocumab group versus 1 patient (0.6%) in the placebo group. All patients were with T2DM. No CPK increase >10 ULN was reported.

Numerical, minor differences were observed in the proportions of patients with mild, moderate, or severe decrease in glomerular filtration rate (GFR) during the treatment period, regardless of the baseline status: mild, moderate and severe decreases in GFR in 49.7%, 28.1% and 3.8% of patients, respectively in the alirocumab group, and in 50.6%, 24.4% and 3.6% of patients, respectively in the placebo group. Similarly, blood creatinine increases (30% and <100%) were measured in 13 (3.8%) patients in the alirocumab group versus 5 patients (3.0%) in the placebo group. No patient had increase in blood creatinine >=100%. There was no meaningful difference in renal function.

No meaningful differences for vital signs were observed between treatment groups.

### Example 3: Analysis of Individuals with Type 2 Diabetes Mellitus and ASCVD from Odyssey DM-Insulin Clinical Trial

Individuals with diabetes often have high levels of atherogenic lipoproteins and cholesterol reflected by elevated low-density lipoprotein cholesterol (LDL-C), non-high-density lipoprotein cholesterol (non-HDL-C), apolipoprotein B (ApoB), and low-density lipoprotein particle number (LDL-PN). Presence of atherosclerotic cardiovascular disease (ASCVD) increases the risk of future cardiovascular events.

In this analysis, we evaluated the efficacy and safety of alirocumab among individuals with T2DM, high LDL-C, or non-HDL-C, and established ASCVD receiving maximally tolerated statin in the DM-Insulin study. DM-Insulin study participants with ASCVD and T1 DM were not included in this analysis due to the low number of individuals in this group (alirocumab: n=11; placebo: n=5). As used in this Example, ASCVD was defined as coronary heart disease (CHD; acute and silent myocardial infarction (MI), and unstable angina), ischemic stroke, or peripheral arterial disease.

Baseline and efficacy data were analyzed according to studies. Efficacy analysis included Week 24 percentage reduction from baseline in non-HDL-C, LDL-C, ApoB, and LDL-PN, and percentage of individuals achieving non-HDL-C<100 mg/dL (<2.59 mmol/L), LDL-C<70 mg/dL (<1.81 mmol/L), and ApoB <80 mg/dL at Week 24. Intention-to-treat (ITT) analysis included all randomized individuals with a baseline LDL-C value and at least one LDL-C value up to Week 24.

This analysis included 177 DM-Insulin individuals with established ASCVD and T2DM (Table 16).

TABLE 16
Baseline characteristics (randomized population)

| | Alirocumab | | Placebo | |
|---|---|---|---|---|
| | n = 119) | | (n = 58) | |
| Age, years, mean (SD) | 66.2 | (8.7) | 64.9 | (8.9) |
| Gender, male, n (%) | 79 | (66.4) | 32 | (55.2) |
| BMI, kg/m², mean (SD) | 32.6 | (4.5) | 33.4 | (5.8) |
| CHD, n (%) | 102 | (85.7) | 51 | (87.9) |
| Acute MI | 59 | (49.6) | 18 | (31.0) |
| Silent MI | 4 | (3.4) | 4 | (6.9) |
| Unstable angina | 15 | (12.6) | 4 | (6.9) |
| Coronary revascularization | 80 | (67.2) | 37 | (63.8) |
| procedure | | | | |
| Other clinical significant CHD^{†} | 31 | (26.1) | 15 | (25.9) |
| Ischemic stroke, n (%) | 27 | (22.7) | 9 | (15.5) |
| PAD, n (%) | 13 | (10.9) | 6 | (10.3) |
| HTN,^{‡} n (%) | 105 | (88.2) | 53 | (91.4) |
| CKD,^{§} n (%) | 37 | (31.1) | 13 | (22.4) |
| Diabetes target organ damage,^{∥} | 60 | (50.4) | 28 | (48.3) |
| n (%) | | | | |
| Statin, n (%) | 88 | (73.9) | 39 | (67.2) |
| Any LLT other than statin, n (%) | 34 | (28.6) | 11 | (19.0) |
| HbA1c, %, mean (SD) | 7.5 | (0.9) | 7.4 | (1.0) |
| FPG, mg/dL [mmol/L], mean (SD) | 162.6 | (52.5) | 146.7 | (45.2) |
| | [9.0 | (2.9)] | [8.1 | (2.5)] |
| Insulin, n (%) | 119 | (100) | 57 | (98.3)^{¶} |
| Non-insulin GLT, n (%) | | | | |
| Biguanides | 57 | (47.9) | 33 | (56.9) |
| Sulfonylureas | 11 | (9.2) | 7 | (12.1) |
| DPP-4 inhibitor | 21 | (17.6) | 7 | (12.1) |
| GLP-1 receptor agonist | 11 | (9.2) | 8 | (13.8) |
| SGLT2 inhibitor | 10 | (8.4) | 11 | (19.0) |
| Lipids, mg/dL [mmol/L], | | | | |
| mean (SD) | | | | |
| Non-HDL-C | 142.8 | (41.5) | 147.0 | (54.9) |
| | [3.70 | (1.08)] | [3.81 | (1.42)] |
| LDL-C | 107.2 | (35.1) | 111.9 | (46.4) |
| | [2.78 | (0.91)] | [2.90 | (1.20)] |
| ApoB | 96.4 | (25.1) | 98.7 | (32.0) |
| LDL-PN, nmol/L, mean (SD) | 1339.5 | (408.5) | 1425.0 | (467.9) |

^{†}Diagnosis by invasive/non-invasive testing.
^{‡}Includes patients with established HTN on anti-HTN medication.
^{§}Defined as estimated glomerular filtration rate 15-60 mL/min/1.73 m².
^{∥}Defined as microalbuminuria, macroalbuminuria, retinopathy, and/or CKD.
^{¶}One individual in the placebo group was not receiving insulin at the time of randomization, and remained without insulin treatment for the duration of the study.
BMI, body mass index; DPP4, dipeptidyl peptidase 4; GLP-1, glucagon-like petite 1; GLT, glucose-lowering treatment; FPG, fasting plasma glucose; HbA1c, glycated hemoglobin; HTN, hypertension; SGLT2, sodium/glucose contransporter 2.

Regardless of treatment allocation, 89.3% of individuals analyzed had a history of hypertension, and 28.2% had chronic kidney disease (CKD), on top of ASCVD. In total, 20.3% demonstrated a history of ischemic stroke, and 10.7% had peripheral arterial disease (PAD). At baseline, the mean (standard deviation [SD]) non-HDL-C level was 144.2 (46.2) mg/dL [3.73 (1.20) mmol/L]; the mean LDL-C level was 108.7 (39.1) mg/dL [2.82 (1.01) mmol/L], regardless of treatment allocation.

### Efficacy

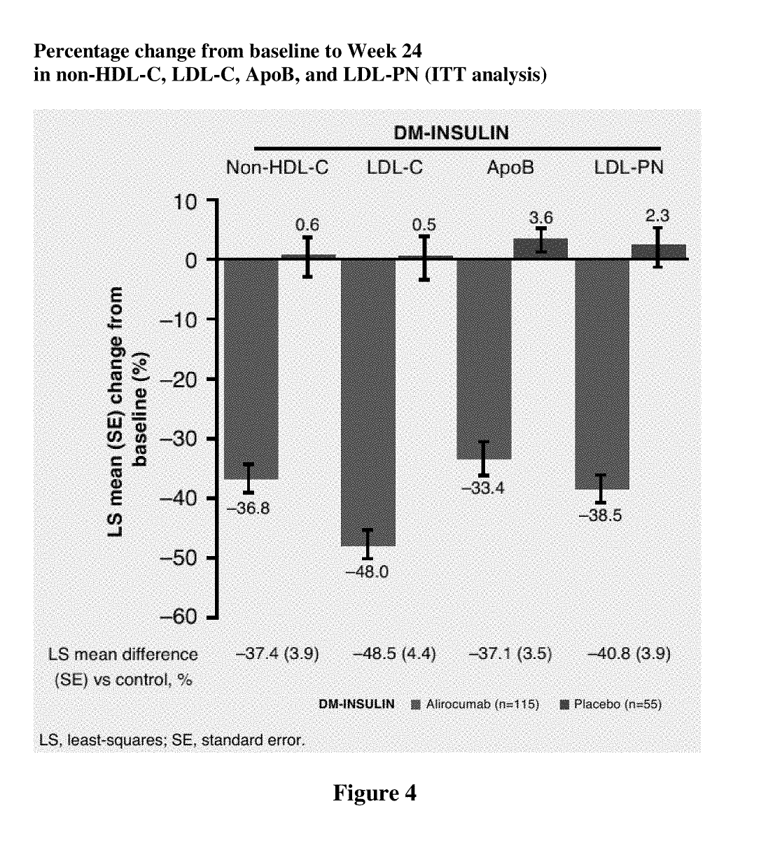

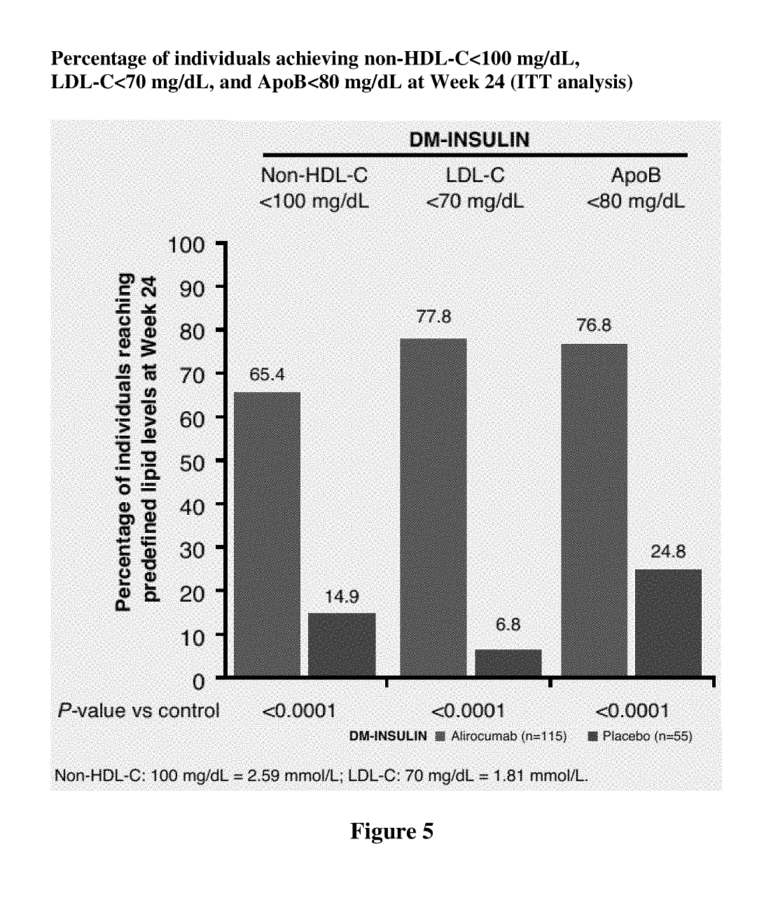

Alirocumab reduced non-HDL-C, ApoB, LDL-PN, and LDL-C from baseline at Week 24 versus control (FIG. 4). At Week 24, a significantly greater proportion of individuals achieved non-HDL-C<100 mg/dL (<2.59 mmol/L), LDL-C<70 mg/dL (<1.81 mmol/L), and ApoB <80 mg/dL versus control (all P<0.0001; FIG. 5).

### Safety

The safety analysis was conducted in a pooled population of individuals with T2DM, high LDL-C or non-HDL-C, and established ASCVD receiving maximally tolerated statin in the DM-Insulin study, and individuals with T1 DM or T2DM, high LDL-C or non-HDL-C, and established ASCVD receiving maximally tolerated statin in the DM-Dyslipidemia study (see Chan et al. (2017) Ann Transl Med. 5(23):477, which is incorporated by reference herein in its entirety). In total, 66.4% (alirocumab) and 67.0% (control) of individuals reported treatment-emergent adverse events (TEAEs; Table 17). The adverse event pattern was similar in both groups. Mean (SD) levels of HbA1c were similar in each treatment group at baseline (alirocumab: 7.3 [0.9]%; control: 7.3 [0.9]%) and Week 24 (alirocumab: 7.6 [1.2]%; control: 7.5 [1.2]%; safety analysis). FPG levels were also similar regardless of treatment allocation at baseline (alirocumab: 154.2 [47.9] mg/dL, 8.6 [2.7] mmol/L; control: 149.5 [43.7] mg/dL, 8.3 [2.4] mmol/L) and at Week 24 (alirocumab: 164.7 [54.9] mg/dL, 9.1 [3.0] mmol/L; control: 159.4 [48.4] mg/dL, 8.9 [2.7] mmol/L; safety analysis).

TABLE 17
Safety summary

| | Alrocumab | | Control | |
|---|---|---|---|---|
| n (%) | (n = 213) | | (n = 104) | |
| TEAEs | 142 | (66.7) | 70 | (67.3) |
| Treatment-emergent SAEs | 28 | (13.1) | 10 | (9.6) |
| TEAEs leading to death | 1 | (0.5) | 1 | (1.0) |
| TEAEs leading to permanent treatment | 13 | (6.1) | 2 | (2.3) |
| discontinuation | | | | |
| TEAEs occurring in ≥2% of individuals | | | | |
| by preferred term | | | | |
| Urinary tract infection | 8 | (3.8) | 6 | (5.8) |
| Diarrhea | 8 | (3.8) | 6 | (5.8) |
| Nasopharyngitis | 6 | (2.8) | 5 | (4.8) |
| Influenza | 7 | (3.3) | 4 | (3.8) |
| Hypertension | 8 | (3.8) | 4 | (3.8) |
| Headache | 7 | (3.3) | 1 | (1.0) |
| Musculoskeletal pain | 7 | (3.3) | 3 | (2.9) |
| Arthralgia | 3 | (1.4) | 3 | (2.9) |
| Bronchitis | 3 | (1.4) | 3 | (2.9) |
| Cough | 1 | (0.5) | 3 | (2.9) |
| Fatigue | 5 | (2.3) | 3 | (2.9) |
| Hyperglycaemia | 0 | (0.0) | 3 | (2.9) |
| Hypotension | 2 | (0.9) | 3 | (2.9) |
| Nausea | 4 | (1.9) | 3 | (2.9) |
| Pain in extremity | 4 | (1.9) | 3 | (2.9) |
| Back pain | 6 | (2.8) | 2 | (1.9) |
| Dizziness | 6 | (2.8) | 3 | (2.9) |
| Cataract | 5 | (2.3) | 1 | (1.0) |
| Myalgia | 5 | (2.3) | 1 | (1.0) |

SAE, serious adverse event.

### CONCLUSIONS

Among individuals with T2DM and ASCVD who had high LDL-C levels despite maximally tolerated statins, alirocumab significantly reduced atherogenic cholesterol content and LDL-PN versus control.

1. A method for treating hypercholesterolemia in a patient with type 1 diabetes mellitus (T1DM), the method comprising:
(a) selecting a high cardiovascular risk patient receiving insulin therapy that has (i) T1DM, and
(ii) hypercholesterolemia not adequately controlled by maximally tolerated statin therapy; and
(b) administering to the patient 75 mg, 150 mg or 300 mg of an antibody or an antigen-binding fragment thereof which specifically binds human proprotein convertase subtilisin/kexin type 9 (PCSK9), wherein the antibody or antigen-binding fragment thereof
comprises the three heavy chain CDRs set forth in SEQ ID NOs: 2, 3, and 4, and the three light chain CDRs set forth in SEQ ID NOs: 7, 8, and 10, and wherein the patient receives a concomitant insulin therapy.

2. The method of claim 1, wherein
(a) the 75 mg of the antibody or antigen binding fragment is administered to the patient every two weeks;
(b) the 150 mg of the antibody or antigen binding fragment is administered to the patient every two weeks; or
(c) the 300 mg of the antibody or antigen binding fragment is administered to the patient every four weeks.

3. (canceled)

4. (canceled)

5. (canceled)

6. The method of claim 1, wherein the antibody or antigen-binding fragment thereof comprises a heavy chain variable region (HCVR) having the amino acid sequence of SEQ ID NO: 1 and a light chain variable region (LCVR) having the amino acid sequence of SEQ ID NO: 6.

7. (canceled)

8. (canceled)

9. The method of claim 1, further comprising:
(c) administering to the patient one or more following doses of 75 mg of the antibody or antigen-binding fragment thereof about every two weeks if the LDL-C level in the patient is lower than a threshold level, or administering one or more following doses of 150 mg of the antibody or antigen-binding fragment thereof about every two weeks if the LDL-C level in the patient is greater than or equal to the threshold level.

10. The method of claim 1, further comprising:
(c) administering to the patient one or more following doses of 300 mg of the antibody or antigen-binding fragment thereof about every four weeks if the LDL-C level in the patient is lower than a threshold level, or administering one or more following doses of 150 mg of the antibody or antigen-binding fragment thereof about every two weeks if the LDL-C level in the patient is greater than or equal to the threshold level.

11. The method of claim 9, wherein the threshold level is 70 mg/dL.

12. The method of claim 1, wherein the antibody or antigen-binding fragment thereof is administered subcutaneously.

13. The method of claim 1, wherein the patient further receives a concomitant lipid-modifying therapy (LMT).

14. The method of claim 13, wherein the LMT is selected from the group consisting of a statin, a cholesterol absorption inhibitor, a fibrate, niacin, an omega-3 fatty acid, and a bile acid sequestrant.

15. The method of claim 14, wherein the LMT is a statin therapy.

16. The method of claim 15, wherein the statin is selected from the group consisting of atorvastatin, rosuvastatin, simvastatin, pravastatin, lovastatin, fluvastatin, pitavastatin, and cerivastatin.

17. The method of claim 15, wherein the statin therapy is a maximally tolerated statin therapy.

18. The method of claim 15, wherein the cholesterol absorption inhibitor is ezetimibe.

19. The method of claim 1, wherein the patient is intolerant to a statin.

20. The method of claim 1, wherein the insulin therapy is selected from the group consisting of human insulin, Insulin glargine, insulin glulisine, insulin detemir, insulin lispro, insulin degludec, insulin aspart, and basal insulin.

21. The method of claim 20, wherein the patient receives a concomitant anti-diabetic therapy in addition to insulin therapy.

22. The method of claim 21, where the additional concomitant anti-diabetic therapy is selected from the group consisting of a glucagon like peptide 1 (GLP-1) therapy, a gastrointestinal peptide, a glucagon receptor agonist or antagonist, a glucose-dependent insulinotropic polypeptide (GIP) receptor agonist or antagonist, a ghrelin antagonist or inverse agonist, xenin, a xenin analogue, a biguanide, a sulfonylurea, a meglitinide, a thiazolidinedione, a DPP-4 inhibitor, an alpha-glucosidase inhibitor, a sodium-dependent glucose transporter 2 (SGLT-2) inhibitor, an SGLT-1 inhibitor, a peroxisome proliferator-activated receptor (PPAR−) (alpha, gamma or alpha/gamma) agonist or modulator, amylin, an amylin analogue, a G-protein coupled receptor 119 (GPR119) agonist, a GPR40 agonist, a GPR120 agonist, a GPR142 agonist, a systemic or low-absorbable TGR5 agonist, a diabetes immunotherapeutic, an anti-inflammatory agents for the treatment of the metabolic syndrome and diabetes, an adenosine monophosphate-activated protein kinase (AMPK) stimulant, an inhibitor of 11-beta-hydroxysteroid dehydrogenase 1, an activator of glucokinase, an inhibitor of diacylglycerol O-acyltransferase (DGAT), a modulator of glucose transporter-4, a somatostatin receptor 3 agonist, a lipid lowering agent, and a combination thereof.

23. The method of claim 1, wherein the antibody or antigen-binding fragment thereof:
(a) reduces the LDL-C level of the patient by at least 40%;
(b) reduces the non-HDL-C level of the patient by at least 35%;
(c) reduces the apolipoprotein C3 (ApoC3) level of the patient;
(d) reduces the number and/or size of lipoprotein particles in the patient
(e) does not affect the hemoglobin A1c (HbA1c) level of the patient; and/or
(f) does not affect the fasting plasma glucose (FPG) level of the patient.

24. (canceled)

25. (canceled)

26. (canceled)

27. (canceled)

28. (canceled)

29. A method for treating hypercholesterolemia in a patient with type 2 diabetes mellitus (T2DM), the method comprising:
(a) selecting a high cardiovascular risk patient receiving insulin therapy that has (i) T2DM, and
(ii) hypercholesterolemia not adequately controlled by maximally tolerated statin therapy; and
(b) administering to the patient 75 mg, 150 mg or 300 mg of an antibody or an antigen-binding fragment thereof which specifically binds human proprotein convertase subtilisin/kexin type 9 (PCSK9), wherein the antibody or antigen-binding fragment thereof comprises the three heavy chain CDRs set forth in SEQ ID NOs: 2, 3, and 4, and the three light chain CDRs set forth in SEQ ID NOs: 7, 8, and 10, and wherein the patient receives a concomitant insulin therapy,

30. The method of claim 29, wherein
(a) the 75 mg of the antibody or antigen binding fragment is administered to the patient every two weeks;
(b) the 150 mg of the antibody or antigen binding fragment is administered to the patient every two weeks; or
(c) the 300 mg of the antibody or antigen binding fragment is administered to the patient every four weeks.

31. (canceled)

32. (canceled)

33. (canceled)

34. The method of claim 29, wherein the antibody or antigen-binding fragment thereof comprises a heavy chain variable region (HCVR) having the amino acid sequence of SEQ ID NO: 1 and a light chain variable region (LCVR) having the amino acid sequence of SEQ ID NO: 6.

35. (canceled)

36. (canceled)

37. The method of claim 29, further comprising:
(c) administering to the patient one or more following doses of 75 mg of the antibody or antigen-binding fragment thereof about every two weeks if the LDL-C level in the patient is lower than the threshold level, or administering one or more following doses of 150 mg of the antibody or antigen-binding fragment thereof about every two weeks if the LDL-C level in the patient is greater than or equal to the threshold level.

38. The method of claim 29, further comprising:
(c) administering to the patient one or more following doses of 300 mg of the antibody or antigen-binding fragment thereof about every four weeks if the LDL-C level in the patient is lower than a threshold level, or administering one or more following doses of 150 mg of the antibody or antigen-binding fragment thereof about every two weeks if the LDL-C level in the patient is greater than or equal to the threshold level.

39. The method of claim 37, wherein the threshold level is 70 mg/dL.

40. The method of claim 29, wherein the antibody or antigen-binding fragment thereof is administered subcutaneously.

41. The method of claim 29, wherein
(a) the patient further receives a concomitant lipid-modifying therapy (LMT);
(b) the patient further receives a concomitant lipid-modifying therapy (LMT) selected from the group consisting of a statin, a cholesterol absorption inhibitor, a fibrate, niacin, an omega-3 fatty acid, and a bile acid sequestrant,
(c) the patient further receives a concomitant statin therapy;
(d) the patient further receives a concomitant statin therapy selected from the group consisting of atorvastatin, rosuvastatin, simvastatin, pravastatin, lovastatin, fluvastatin, pitavastatin, and cerivastatin;
(e) the patient further receives a concomitant statin therapy, wherein the statin therapy is a maximally tolerated statin therapy; and/or
(f) the patient further receives a cholesterol absorption inhibitor, wherein the cholesterol absorption inhibitor is ezetimibe.

42. (canceled)

43. (canceled)

44. (canceled)

45. (canceled)

46. (canceled)

47. The method of claim 29, wherein the patient is intolerant to a statin.

48. The method of claim 29, wherein
(a) the insulin therapy is selected from the group consisting of human insulin, Insulin glargine, insulin glulisine, insulin detemir, insulin lispro, insulin degludec, insulin aspart, and basal insulin;
(b) the patient receives a concomitant anti-diabetic therapy in addition to insulin therapy; and/or
(c) the patient receives a concomitant anti-diabetic therapy in addition to insulin therapy selected from the group consisting of a glucagon like peptide 1 (GLP-1) therapy, a gastrointestinal peptide, a glucagon receptor agonist or antagonist, a glucose-dependent insulinotropic polypeptide (GIP) receptor agonist or antagonist, a ghrelin antagonist or inverse agonist, xenin, a xenin analogue, a biguanide, a sulfonylurea, a meglitinide, a thiazolidinedione, a DPP-4 inhibitor, an alpha-glucosidase inhibitor, a sodium-dependent glucose transporter 2 (SGLT-2) inhibitor, an SGLT-1 inhibitor, a peroxisome proliferator-activated receptor (PPAR−)(alpha, gamma or alpha/gamma) agonist or modulator, amylin, an amylin analogue, a G-protein coupled receptor 119 (GPR119) agonist, a GPR40 agonist, a GPR120 agonist, a GPR142 agonist, a systemic or low-absorbable TGR5 agonist, a diabetes immunotherapeutic, an anti-inflammatory agents for the treatment of the metabolic syndrome and diabetes, an adenosine monophosphate-activated protein kinase (AMPK) stimulant, an inhibitor of 11-beta-hydroxysteroid dehydrogenase 1, an activator of glucokinase, an inhibitor of diacylglycerol O-acyltransferase (DGAT), a modulator of glucose transporter-4, a somatostatin receptor 3 agonist, a lipid lowering agent, and a combination thereof.

49. (canceled)

50. (canceled)

51. The method of claim 29, wherein the antibody or antigen-binding fragment thereof
(a) reduces the LDL-C level of the patient by at least 40%;
(b) reduces the non-HDL-C level of the patient by at least 35%;
(c) reduces the apolipoprotein C3 (ApoC3) level of the patient;
(d) reduces the number and/or size of lipoprotein particles in the patient;
(e) does not affect the hemoglobin A1c (HbA1c) level of the patient; and/or
(f) does not affect the fasting plasma glucose (FPG) level of the patient.

52. (canceled)

53. (canceled)

54. (canceled)

55. (canceled)

56. The method of claim 29, wherein the method comprises:
(a) selecting a high cardiovascular risk patient receiving insulin therapy that has (i) T2DM, and
(ii) hypercholesterolemia not adequately controlled by maximally tolerated statin therapy;
(b) administering every two weeks to the patient 75 mg of an antibody or an antigen-binding fragment thereof which specifically binds human proprotein convertase subtilisin/kexin type 9 (PCSK9); and
(c) administering to the patient one or more following doses of 75 mg of the antibody or antigen-binding fragment thereof about every two weeks if the LDL-C level in the patient is lower than 70 mg/dL, or administering one or more following doses of 150 mg of the antibody or antigen-binding fragment thereof about every two weeks if the LDL-C level in the patient is greater than or equal to 70 mg/dL,
wherein the antibody or antigen-binding fragment thereof comprises an HCVR having the amino acid sequence of SEQ ID NO: 1 and an LCVR having the amino acid sequence of SEQ ID NO: 6, and wherein the patient receives a concomitant insulin therapy.

57. A method for treating hypercholesterolemia in a patient with T2DM and atherosclerotic cardiovascular disease (ASCVD), the method comprising:
(a) selecting a high cardiovascular risk patient receiving insulin therapy that has (i) T2DM, (ii) ASCVD, and (iii) hypercholesterolemia not adequately controlled by maximally tolerated statin therapy; and
(b) administering to the patient 75 mg, 150 mg or 300 mg of an antibody or an antigen-binding fragment thereof which specifically binds human proprotein convertase subtilisin/kexin type 9 (PCSK9), wherein the antibody or antigen-binding fragment thereof comprises the three heavy chain CDRs set forth in SEQ ID NOs: 2, 3, and 4, and the three light chain CDRs set forth in SEQ ID NOs: 7, 8, and 10, and wherein the patient receives a concomitant insulin therapy.

58. (canceled)

59. (canceled)

60. The method of claim 57, wherein
(a) the 75 mg of the antibody or antigen binding fragment is administered to the patient every two weeks;
(b) the 150 mg of the antibody or antigen binding fragment is administered to the patient every two weeks; or
(c) the 300 mg of the antibody or antigen binding fragment is administered to the patient every four weeks.

61. (canceled)

62. (canceled)

63. (canceled)

64. The method of claim 57, wherein the antibody or antigen-binding fragment thereof comprises a heavy chain variable region (HCVR) having the amino acid sequence of SEQ ID NO: 1 and a light chain variable region (LCVR) having the amino acid sequence of SEQ ID NO: 6.

65. (canceled)

66. (canceled)

67. The method of claim 57, further comprising:
(c) administering to the patient one or more following doses of 75 mg of the antibody or antigen-binding fragment thereof about every two weeks if the LDL-C level in the patient is lower than the threshold level, or administering one or more following doses of 150 mg of the antibody or antigen-binding fragment thereof about every two weeks if the LDL-C level in the patient is greater than or equal to the threshold level.

68. The method of claim 57, further comprising:
(c) administering to the patient one or more following doses of 300 mg of the antibody or antigen-binding fragment thereof about every four weeks if the LDL-C level in the patient is lower than a threshold level, or administering one or more following doses of 150 mg of the antibody or antigen-binding fragment thereof about every two weeks if the LDL-C level in the patient is greater than or equal to the threshold level.

69. The method of claim 67, wherein the threshold level is 70 mg/dL.

70. The method of claim 57, wherein the antibody or antigen-binding fragment thereof is administered subcutaneously.

71. The method of claim 57, wherein
(a) the patient further receives a concomitant lipid-modifying therapy (LMT);
(b) the patient further receives a concomitant lipid-modifying therapy (LMT) selected from the group consisting of a statin, a cholesterol absorption inhibitor, a fibrate, niacin, an omega-3 fatty acid, and a bile acid sequestrant;
(c) the patient further receives a concomitant statin therapy;
(d) the patient further receives a concomitant statin therapy selected from the group consisting of atorvastatin, rosuvastatin, simvastatin, pravastatin, lovastatin, fluvastatin, pitavastatin, and cerivastatin;
(e) the patient further receives a concomitant statin therapy, wherein the statin therapy is a maximally tolerated statin therapy; and/or
(f) the patient further receives a cholesterol absorption inhibitor, wherein the cholesterol absorption inhibitor is ezetimibe.

72. (canceled)

73. (canceled)

74. (canceled)

75. (canceled)

76. The method of claim 57, wherein the patient is intolerant to a statin.

77. The method of claim 57, wherein the insulin therapy is selected from the group consisting of human insulin, Insulin glargine, insulin glulisine, insulin detemir, insulin lispro, insulin degludec, insulin aspart, and basal insulin
(a) the insulin therapy is selected from the group consisting of human insulin, Insulin glargine, insulin glulisine, insulin detemir, insulin lispro, insulin degludec, insulin aspart, and basal insulin;
(b) the patient receives a concomitant anti-diabetic therapy in addition to insulin therapy; and/or
(c) the patient receives a concomitant anti-diabetic therapy in addition to insulin therapy selected from the group consisting of a glucagon like peptide 1 (GLP-1) therapy, a gastrointestinal peptide, a glucagon receptor agonist or antagonist, a glucose-dependent insulinotropic polypeptide (GIP) receptor agonist or antagonist, a ghrelin antagonist or inverse agonist, xenin, a xenin analogue, a biguanide, a sulfonylurea, a meglitinide, a thiazolidinedione, a DPP-4 inhibitor, an alpha-glucosidase inhibitor, a sodium-dependent glucose transporter 2 (SGLT-2) inhibitor, an SGLT-1 inhibitor, a peroxisome proliferator-activated receptor (PPAR−)(alpha, gamma or alpha/gamma) agonist or modulator, amylin, an amylin analogue, a G-protein coupled receptor 119 (GPR119) agonist, a GPR40 agonist, a GPR120 agonist, a GPR142 agonist, a systemic or low-absorbable TGR5 agonist, a diabetes immunotherapeutic, an anti-inflammatory agents for the treatment of the metabolic syndrome and diabetes, an adenosine monophosphate-activated protein kinase (AMPK) stimulant, an inhibitor of 11-beta-hydroxysteroid dehydrogenase 1, an activator of glucokinase, an inhibitor of diacylglycerol O-acyltransferase (DGAT), a modulator of glucose transporter-4, a somatostatin receptor 3 agonist, a lipid lowering agent, and a combination thereof.

78. (canceled)

79. (canceled)

80. The method of claim 57, wherein the antibody or antigen-binding fragment thereof
(a) reduces the LDL-C level of the patient by at least 40%;
(b) reduces the non-HDL-C level of the patient by at least 35%;
(c) reduces the apolipoprotein C3 (ApoC3) level of the patient;
(d) reduces the number and/or size of lipoprotein particles in the patient
(e) does not affect the hemoglobin A1c (HbA1c) level of the patient; and/or
(f) does not affect the fasting plasma glucose (FPG) level of the patient.

81. (canceled)

82. (canceled)

83. (canceled)

84. (canceled)

85. The method of claim 57, wherein the method comprises:
(a) selecting a high cardiovascular risk patient receiving insulin therapy that has (i) T2DM, (ii) ASCVD, and (iii) hypercholesterolemia not adequately controlled by maximally tolerated statin therapy;
(b) administering every two weeks to the patient 75 mg of an antibody or an antigen-binding fragment thereof which specifically binds human proprotein convertase subtilisin/kexin type 9 (PCSK9); and
(c) administering to the patient one or more following doses of 75 mg of the antibody or antigen-binding fragment thereof about every two weeks if the LDL-C level in the patient is lower than 70 mg/dL, or administering one or more following doses of 150 mg of the antibody or antigen-binding fragment thereof about every two weeks if the LDL-C level in the patient is greater than or equal to 70 mg/dL,
wherein the antibody or antigen-binding fragment thereof comprises an HCVR having the amino acid sequence of SEQ ID NO: 1 and an LCVR having the amino acid sequence of SEQ ID NO: 6, and wherein the patient receives a concomitant insulin therapy.

86. The method of claim 10, wherein the threshold level is 70 mg/dL.

87. The method of claim 38, wherein the threshold level is 70 mg/dL.

88. The method of claim 68, wherein the threshold level is 70 mg/dL.
